(19) United States
(12) Patent Application Publication
(10) Pub. No.: US 2018/0021294 A1
(43) Pub. Date: Jan. 25, 2018

(54) Ephrin Receptor A2 (EPHA2)-Targeted Docetaxel-Generating Nano-Liposome Compositions

(71) Applicant: Merrimack Pharmaceuticals, Inc., Cambridge, MA (US)

(72) Inventors: Daryl C. Drummond, Lincoln, MA (US); Dmitri B. Kirpotin, Revere, MA (US); Zhaohua R. Huang, Braintree, MA (US); Suresh K. Tipparaju, Arlington, MA (US); Charles Noble, San Francisco, CA (US)

(21) Appl. No.: 15/460,280

(22) Filed: Mar. 16, 2017

Related U.S. Application Data
(60) Provisional application No. 62/464,538, filed on Feb. 28, 2017.
(60) Provisional application No. 62/419,012, filed on Nov. 8, 2016.
(60) Provisional application No. 62/338,052, filed on May 18, 2016.
(60) Provisional application No. 62/322,940, filed on Apr. 15, 2016.
(60) Provisional application No. 62/309,222, filed on Mar. 16, 2016.

(51) Int. Cl. A61K 31/337; A61K 9/127

(52) U.S. Cl. CPC A61K 31/337; A61K 9/1272

(57) ABSTRACT

EphA2-targeted immunoliposomes for delivering docetaxel are useful in the treatment of certain types of cancer. The immunoliposomes can include an EphA2 targeting moiety (e.g., a scFv) and encapsulate a docetaxel prodrug in a stable salt form within a liposome having an average size of about 100 nm. Novel docetaxel prodrugs suitable for loading into nanoliposomes (including immunoliposomes) are provided, along with novel and other useful EphA2 targeting moieties for preparation of EphA2-targeted doxorubicin-generating immunoliposome therapies. Pharmaceutical compositions can be prepared that include nanoliposomes encapsulating one or more docetaxel prodrugs, and/or immunoliposomes or nanoparticles comprising an EphA2 binding moiety and encapsulating one or more docetaxel prodrugs. The pharmaceutical compositions are useful for administration to a patient for the treatment of cancer.

### CROSS-REFERENCE

This patent application claims priority to each of the following pending U.S. provisional patent applications, each incorporated herein by reference is their entirety: 62/309,222 (filed Mar. 16, 2016), 62/322,940 (filed Apr. 15, 2016), 62/338,052 (filed May 18, 2016), 62/419,012 (filed Nov. 8, 2016) and 62/464,538 (filed Feb. 28, 2017).

### SEQUENCE LISTING

Incorporated by reference in its entirety is a computer-readable sequence listing submitted concurrently herewith and identified as follows: One 48.0 KB ASCII (Text) file named “1106sequence_ST25.txt.”

### TECHNICAL FIELD

This disclosure relates to nanoliposomes that bind to Ephrin receptor A2 (EphA2) and deliver docetaxel, useful in the treatment of EphA2-positive cancer.

### BACKGROUND

Ephrin receptors are cell to cell adhesion molecules that mediate signaling and are implicated in neuronal repulsion, cell migration and angiogenesis. EphA2 is part of the Ephrin family of cell-cell junction proteins highly overexpressed in several solid tumors. Ephrin receptor A2 (EphA2) is overexpressed in certain solid tumors, and is associated with poor prognosis in certain cancer conditions. The Eph receptors are comprised of a large family of tyrosine kinase receptors divided into two groups (A and B) based upon homology of the N-terminal ligand binding domain. The Eph receptors are involved several key signaling pathways that control cell growth, migration and differentiation. These receptors are unique in that their ligands bind to the surface of neighboring cells. The Eph receptors and their ligands display specific patterns of expression during development. For example, the EphA2 receptor is expressed in the nervous system during embryonic development and also on the surface of proliferating epithelial cells in adults. EphA2 also plays an important role in angiogenesis and tumor vascularization, mediated through the ligand ephrin A1. In addition, EphA2 is overexpressed in a variety of human tumors including urothelial, breast, gastric/GEJ, head and neck, non-small cell lung, ovarian, pancreatic and prostate carcinomas. Expression of EphA2 can also be detected in tumor blood vessels as well.

While taxanes are widely used to treat solid tumors either in the curative or palliative setting, in first or later lines of therapy, analysis of docetaxel dose-response relationship strongly suggests that a higher dose would lead to high response and will also lead to higher toxicity. This is likely related to the lack of organ and cellular specificity of docetaxel leading to high exposures in normal tissues and the relatively short circulation half-life which indirectly requires higher doses. There is a need for therapeutic taxane compositions permitting improved treatment of certain cancer conditions.

### SUMMARY

Applicants have discovered novel EphA2 targeted nanoliposomes for delivering docetaxel to tumors, capable of leveraging organ specificity through enhanced permeability and retention, as well as leveraging cellular specificity through an EphA2 targeting moiety covalently bound to the nanoliposome membrane.

With the goal of addressing the pharmacokinetic limitations of free docetaxel and the lack of cellular specificity, we developed novel docetaxel-based nanoliposomes, targeted against Ephrin receptor A2 (EphA2) which is overexpressed in a wide range of tumors. These EphA2-targeted docetaxel-generating liposomes provide sustained release of docetaxel following accumulation in solid tumors. Preclinical models have demonstrated that EphA2-targeted docetaxel-generating liposomes leverage tumor-specific accumulation through the enhanced permeability and retention effect, and cellular specificity through active targeting of EphA2 with specific scFv antibody fragments conjugated to the surface of the liposomes.

Pharmacokinetic and biodistribution studies were performed in mice and rats to compare EphA2 targeted docetaxel-generating nanoliposomes to free docetaxel. Chronic tolerability studies were performed in rodent and non-rodent models with focus on overall animal health, as well as hematologic toxicities. Several cell-derived models of breast, lung and prostate xenografts were used to evaluate the differences between EphA2-targeted docetaxel-generating liposomes and free (i.e., not in a liposome) docetaxel.

EphA2 targeted docetaxel-generating nanoliposome compositions had a significantly longer half-life than free docetaxel with prolonged exposure at the tumor site. In chronic tolerability studies, certain EphA2 targeted docetaxel-generating nanoliposome compositions were found to be 6-7 times better tolerated than free docetaxel with a maximum tolerated dose of at least 120 mpk (i.e., mg drug per kg animal body weight), compared to 20 mpk for free docetaxel and no detectable hematological toxicity. At equitoxic dosing, certain EphA2 targeted docetaxel-generating nanoliposome compositions 50 mpk showed greater activity than docetaxel 10 mpk in several breast, lung and prostate xenograft models.

We developed a novel EphA2 targeted docetaxel nanoliposome with prolonged circulation time and slow and sustained drug release kinetics, to enable organ and cellular targeting. Certain EphA2 targeted docetaxel-generating nanoliposome compositions were able to overcome hematologic toxicities observed upon treatment with free docetaxel in rodent and non-rodent models. Certain EphA2 targeted docetaxel-generating nanoliposome compositions were also able to induce tumor regression or control tumor growth in several cell derived xenograft models, and was found to be more active than free docetaxel in most models.

Binding of targeted liposomes to cells in vitro was assessed using flow cytometry in a large panel cell lines. 3D spheroids were used to assess targeting as well as liposome penetration. In order to test the effect of targeting on liposome microdistribution in vivo, primary and metastatic tumor bearing animals were injected with a mixture of EphA2-targeted liposome (EphA2-Ls) and non-targeted liposome (NT-Ls) labeled with two different lipophilic fluorescent dyes. Tissues were assessed using fluorescent microscopy. In order to evaluate the contribution of EphA2-targeting to efficacy, four gastric xenograft models were treated with either the EphA2 targeted docetaxel-generating nanoliposome compositions tested or a non-targeted version of the drug, as compared to free docetaxel.

In cell suspension models, we observed a high level of specificity for EphA2-Ls, with more than one hundred-fold increase in liposome cell association. 3D spheroid assays showed that EphA2-Ls binds and penetrates EphA2+ spheroids, while non-targeted liposomes show minimal penetration. Tissue microdistribution analysis in triple negative breast and esophageal tumor models following injection of the EphA2-Ls/NT-Ls mixtures showed a target mediated shift in the microdistribution of liposomes. EphA2-Ls penetrated deeper within the lesions while the NT-Ls deposited at high levels in areas close to the microvasculature. The target mediated shift in microdistribution was also observed in lung metastasis model, with a pattern of distribution that potentially matches disseminated tumor cells. In the same animals, targeting did not affect microdistribution in normal organs such as liver, spleen and skin. Four models of gastric and esophageal cancers were used to test the potential link between cell targeting and tumor growth control. While non-targeted liposomes encapsulating a docetaxel prodrug and EphA2 targeted docetaxel-generating nanoliposome compositions were both able to control tumor growth leading to regression in most models, in three out of four models there was a statistically significant difference between EphA2 targeted docetaxel-generating nanoliposome compositions and non-targeted liposomes encapsulating a docetaxel prodrug at 25 mpk. Biomarker analysis is underway to evaluate the key parameters necessary to mediate targeting.

In conclusion, the data suggests clear evidence of targeting in 2D cell suspension, 3D spheroids, and in primary as well as metastatic tumor models in vivo. In vivo efficacy data showed evidence of the contribution of EphA2 targeting to tumor growth control and regression in several gastric cancer models.

### BRIEF DESCRIPTION OF THE DRAWINGS

FIG. 1A is a schematic of a docetaxel-generating liposome comprising an EphA2 binding moiety (anti-EphA2 scFv PEG-DSPE).

FIG. 1B is a schematic showing the processes of docetaxel prodrug loading into a liposome comprising sucrose octasulfate (SOS) as a trapping agent, and the process of docetaxel generation. The insolubility of the salt in the liposome interior when combined with a low pH environment can stabilize the prodrug to reduce or prevent premature conversion to the active docetaxel.

FIG. 2A is a chemical reaction scheme for the synthesis of certain docetaxel prodrugs.

FIG. 2B is a chart showing selected examples of docetaxel prodrugs.

FIG. 2C is a reaction scheme showing the synthesis of PEG-DSG-E.

FIG. 3A is a schematic showing hydrolysis profiles at 37 degrees C. for preferred docetaxel prodrugs. The hydrolysis profile can be obtained using the method of Example 11.

FIG. 3B is a hydrolysis profile for a certain docetaxel prodrug.

FIG. 3C is a hydrolysis profile for a certain docetaxel prodrug.

FIG. 3D is a hydrolysis profile for a certain docetaxel prodrug.

FIG. 3E is a hydrolysis profile for a certain docetaxel prodrug.

FIG. 3F is a hydrolysis profile for a certain docetaxel prodrug.

FIG. 4A shows various CDR sequences useful in EphA2 binding moieties that can be used to prepare an EphA2 targeted docetaxel-generating nanoliposome composition.

FIG. 4B is an amino acid sequence and corresponding encoding DNA sequence for the scFv that can be used to prepare an EphA2 targeted docetaxel-generating nanoliposome composition. The DNA sequence further encodes an N-terminal leader sequence that is cleaved off by mammalian (e.g., human or rodent) cells expressing the encoded scFv.

FIG. 4C is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4D is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4E is an amino acid sequence and corresponding encoding DNA sequence for the scFv EphA2 binding moiety in an EphA2 targeted docetaxel-generating nanoliposome composition EphA2-ILs, used in Examples 2-9.

FIG. 4F is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4G is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4H is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4I is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4J is an amino acid sequence used in Example 4, and a corresponding encoding DNA sequence.

FIG. 5 is a graph showing DTX (docetaxel) and a docetaxel prodrug of Compound 3 levels in tumor, spleen and liver

FIG. 6A is a graph showing the tolerability of an EphA2-targeted, docetaxel generating immunoliposome (41scFv-ILs-DTXp3) in Swiss Webster mice.

FIG. 6B is a graph showing the tolerability of free docetaxel in Swiss Webster mice.

FIG. 7A is a graph showing the antitumor efficacy an EphA2-targeted, docetaxel generating immunoliposome (40scFv-ILs-DTXp3) in SUM-159 xenograft model.

FIG. 7B is a graph showing the antitumor efficacy an EphA2-targeted, docetaxel generating immunoliposome (40scFv-ILs-DTXp3) in SUM-149 xenograft model.

FIG. 7C is a graph showing antitumor efficacy an EphA2-targeted, docetaxel generating immunoliposome (40scFv-ILs-DTXp3) in MDA-MB-436 xenograft model.

FIG. 7D is a graph showing the tolerability of an EphA2-targeted, docetaxel generating immunoliposome (40scFv-ILs-DTXp3) in SUM-159 xenograft model.

FIG. 7E is a graph showing the tolerability of an EphA2-targeted, docetaxel generating immunoliposome (40scFv-ILs-DTXp3) in SUM-149 xenograft model.

FIG. 7F is a graph showing the tolerability of an EphA2-targeted, docetaxel generating immunoliposome (40scFv-ILs-DTXp3) in MDA-MB-436 xenograft model.

FIG. 8A is a graph showing the antitumor efficacy of a non-targeted docetaxel-generating liposome (NT-Ls-DTXp3) compared to an EphA2-targeted, docetaxel generating immunoliposome (40scFv-ILs-DTXp3) in MDA-MB-436 xenograft model.

FIG. 8B is a graph showing the tolerability of a non-targeted docetaxel-generating liposome (NT-Ls-DTX) compared to an EphA2-targeted, docetaxel generating immunoliposome (40scFv-ILs-DTXp3) in MDA-MB-436 xenograft model.

FIG. 8C is a graph showing the antitumor efficacy of a non-targeted docetaxel-generating liposome (NT-Ls-DTXp3) compared to an EphA2-targeted, docetaxel generating immunoliposome (46scFv-ILs-DTXp3) in OE-19 xenograft model.

FIG. 8D is a graph showing the antitumor efficacy of a non-targeted docetaxel-generating liposome (NT-Ls-DTXp3) compared to an EphA2-targeted, docetaxel generating immunoliposome (46scFv-ILs-DTXp3) in MKN-45 xenograft model.

FIG. 8E is a graph showing the antitumor efficacy of a non-targeted docetaxel-generating liposome (NT-Ls-DTXp3) compared to an EphA2-targeted, docetaxel generating immunoliposome (46scFv-ILs-DTXp3) in OE-21 xenograft model.

FIG. 8F is a graph showing the antitumor efficacy of a non-targeted docetaxel-generating liposome (NT-Ls-DTXp3) compared to an EphA2-targeted, docetaxel generating immunoliposome (46scFv-ILs-DTXp3) as shown by analysis of maximum response and time to regrowth for OE-19, MKN-45, and OE-21 xenograft model.

FIG. 8G is a graph showing the antitumor efficacy of NT-LS-DTXp3 vs 46scFv-ILs-DTXp3 in SK-LMS-1 xenograft model.

FIG. 9 is a graph showing the antitumor efficacy an EphA2-targeted, docetaxel generating immunoliposome (41scFv-ILs-DTXp3) in A549 xenograft model.

FIG. 10A is a graph showing antitumor efficacy an EphA2-targeted, docetaxel generating immunoliposome (46scFv-ILs-DTXp3) in DU-145 xenograft model.

FIG. 10B is a graph showing antitumor efficacy an EphA2-targeted, docetaxel generating immunoliposome (46scFv-ILs-DTXp3) in DU-145 xenograft model.

FIG. 11 is a graph showing EphA2 targeting in 3D spheroids in vitro.

FIG. 12A is a graph of data obtained from animals that received one tail vein injection of either an EphA2-targeted immunoliposome (EphA2-ILs) and a non-targeted liposome (NT-Ls) mixture or 1C1. All tissues were collected 24 h after injection.

FIG. 12B is a graph showing liposomal colocalization analysis of an EphA2 targeted docetaxel-generating nanoliposome composition compared to a non-targeted docetaxel-generating nanoliposome composition.

FIG. 12C is an image showing liposomal ratiometric analysis of EphA2 targeting in esophageal cancer OE-19 xenograft model.

FIG. 12D is a graph showing liposomal ratiometric analysis of EphA2 targeting is a graph of data obtained from animals that received one tail vein injection of an EphA2 targeted docetaxel-generating nanoliposome composition and a non-targeted docetaxel-generating nanoliposome composition mixture. All tissues were collected 24 h after

FIG. 12E is an image showing liposomal ratiometric analysis of EphA2 targeting in orthotopically implanted metastatic model of triple negative breast cancer

FIG. 12F is an image showing liposomal ratiometric analysis of EphA2 targeting in intraveinously injected metastatic model of triple negative breast cancer.

FIG. 13A is a graph showing antitumor efficacy an EphA2 targeted docetaxel-generating nanoliposome composition in orthotopic OVCAR-8 xenograft model

FIG. 13B is a graph showing the lentiviral construct used to generate gaussia luciferase expressing OVCAR-8 cell line

FIG. 13C is a graph showing the transfection protocol used to generate gaussia luciferase expressing OVCAR-8 cell line

FIG. 14A is a graph showing the concentration of docetaxel over time after administering an EphA2-targeted docetaxel-generating liposome to a mouse in Example 13.

FIG. 14B is a graph showing the concentration of lipid over time after administering an EphA2-targeted docetaxel-generating liposome to a mouse in Example 13.

FIG. 14C is a graph showing the docetaxel/lipid ratio over time after administering an EphA2-targeted docetaxel-generating liposome to a mouse in Example 13.

FIG. 15 is a set of graphs showing prothrombin time (PT), activated partial thromboplastin time (APTT), and fibrinogen levels of Sprague Dawley rats administered effects 46scFv-ILs-DTXp3, NT-Ls-DTXp3 or free docetaxel

FIG. 16A is a graph depicting maximal tumor regression in various xenograft models on administration of EphA2 targeted-ILs-DTXp3.

FIG. 16B is a graph depicting maximal tumor regression in various xenograft models administered 10 mg/kg free docetaxel or 50 mg/kg EphA2-targeted-ILs-DTXp3.

### DETAILED DESCRIPTION

EphA2-targeted nanoliposomes can be used to deliver docetaxel (e.g., as an encapsulated docetaxel prodrug) to a cancer cell and/or tumor, leveraging organ specificity through enhanced permeability effect and cellular specificity through EphA2 targeting. We developed a novel EphA2-targeted docetaxel nanoliposome, leveraging organ specificity through enhanced permeability effect and cellular specificity through EphA2 targeting.

“EphA2” refers to Ephrin type-A receptor 2, also referred to as “epithelial cell kinase (ECK),” a receptor tyrosine kinase that can bind and be activated by Ephrin-A ligands. The term “EphA2” can refer to any naturally occurring isoforms of EphA2. The amino acid sequence of human EphA2 is recorded as GenBank Accession No. NP_004422.2.

As used herein, “EphA2 positive” refers to a cancer cell having at least about 3000 EphA2 receptors per cell (or patient with a tumor comprising such a cancer cell). EphA2 positive cells can specifically bind Eph-A2 targeted liposomes per cell. In particular, EphA2 targeted liposomes can specifically bind to EphA2 positive cancer cells having at least about 3000 or more EphA2 receptors per cell.

As used herein, non-targeted liposomes can be designated as “Ls” or “NT-Ls.” Ls (or NT-Ls) can refer to non-targeted liposomes with or without a docetaxel prodrug. “Ls-DTX” refers to liposomes containing any suitable docetaxel prodrug, including equivalent or alternative embodiments to those docetaxel prodrugs disclosed herein. “NT-Ls-DTX” refers to liposomes without a targeting moiety that encapsulate any suitable docetaxel prodrug, including equivalent or alternative embodiments to those docetaxel prodrugs disclosed herein. Examples of non-targeted liposomes including a particular docetaxel prodrug can be specified in the format “Ls-DTXp[y]” or “NT-DTXp[y]” where [y] refers to a particular compound number specified herein. For example, unless otherwise indicated, Ls-DTXp1 is a liposome containing the docetaxel prodrug of compound 1 herein, without an antibody targeting moiety.

As used herein, targeted immunoliposomes can be designated as “ILs.” Recitation of “ILs-DTXp” refers to any embodiments or variations of the targeted docetaxel-generating immunoliposomes comprising a targeting moiety, such as a scFv. The ILs disclosed herein refer to immunoliposomes comprising a moiety for binding a biological epitope, such as an epitope-binding scFv portion of the immunoliposome. Unless otherwise indicated, ILs recited herein refer to EphA2 binding immunoliposomes (alternatively referred to as “EphA2-ILs”). The term “EphA2-ILs” refers herein to immunoliposomes enabled by the present disclosure with a moiety targeted to bind to EphA2. ILs include EphA2-ILs having a moiety that binds to EphA2 (e.g., using any scFv sequences that bind EphA2). Preferred targeted docetaxel-generating immunoliposomes include ILs-DTXp3, ILs-DTXp4, and ILs-DTXp6. Absent indication to the contrary, these include immunoliposomes with an EphA2 binding moiety and encapsulating docetaxel prodrugs of compound 3, compound 4 or compound 6 (respectively). EphA2-ILs can refer to and include immunoliposomes with or without a docetaxel prodrug (e.g., immunoliposomes encapsulating a trapping agent such as sucrose octasulfate without a docetaxel prodrug).

The abbreviation format “[x]scFv-ILs-DTXp[y]” is used herein to describe examples of immune-liposomes (“ILs”) that include a scFv “targeting” moiety having the amino acid sequence specified in a particular SEQ ID NO:[x], attached to a liposome encapsulating or otherwise containing a docetaxel prodrug (“DTXp”) having a particular Compound number ([y]) specified herein. Unless otherwise indicated, the scFv sequences for targeted ILs can bind to the EphA2 target.

The term “NT-Ls” refers to non-targeted liposomes enabled by this disclosure without a targeting moiety. The term “NT-LS-DTXp3” refers to a non-targeted liposomes enabled by this disclosure encapsulating a docetaxel prodrug (“DTX”).

As used herein, the term “mpk” refers to mg per kg body weight in a dose administered to an animal.

Preferably, the immunoliposomes (ILs) or non-targeted liposomes (Ls or NT-LS) comprise a suitable amount of PEG (i.e., PEGylated) attached to one or more components of the liposome vesicle to provide a desired plasma half-life upon administration.

In one embodiment, the invention is an EphA2-targeted docetaxel-generating liposome comprising a docetaxel prodrug encapsulated within a lipid vesicle comprising one or more lipids, a PEG lipid derivative and an EphA2 binding moiety on the outside of the lipid vesicle.

In some embodiments, the EphA2 binding moiety is a scFv moiety covalently bound to a lipid within the lipid vesicle. In some embodiments, the docetaxel prodrug is a compound of Formula (I)

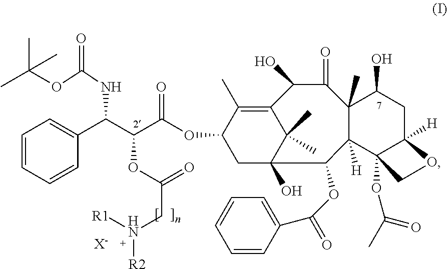

where R1 and R2 are each independently H or lower alkyl, and n is an integer 2-3.

In some embodiments, the EphA2 binding moiety is a scFv moiety comprising the CDRs of SEQ ID NO:40 or SEQ ID NO:41. In some embodiments, the EphA2 binding moiety is a scFv moiety comprising the sequence of SEQ ID NO:41.

In some embodiments, the lipid vesicle comprises sphingomyelin, cholesterol and PEG-DSG. In some embodiments, the lipid vesicle comprises sphingomyelin, cholesterol and PEG-DSG in a weight ratio of about 4.4:1.6:1.

In some embodiments, the liposome encapsulates a docetaxel-generating prodrug of formula (I)

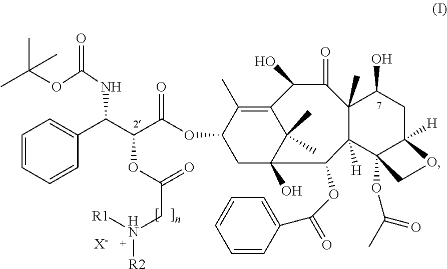

where R1 and R2 are each C₁-C₃ alkyl, and n is 2 or 3. In some embodiments, the docetaxel-generating prodrug encapsulates the compound of formula (I) with sucrose octasulfate. In some embodiments, the docetaxel prodrug is a sucrose octasulfate salt of any one of Compounds 1-3 encapsulated in a liposome.

In some embodiments, the liposome further comprises the scFv moiety covalently bound to PEG-DSPE as scFv-PEG-DSPE in a ratio of about 1:142 by weight with respect to the total sphingomyelin in the liposome.

In some embodiments, the drug-to-phospholipid ratio in the final liposome is greater than 250 g docetaxel equivalents/mol phospholipid, and preferably between 250-350 g/mol when the drug loading is based on docetaxel equivalents.

In some embodiments, the pH of the storage buffer is below neutral, preferably between 4-6.5, and more preferably between 5-6.

In some embodiments, the EphA2 binding moiety binds to the same epitope on EphA2 as an scFv consisting of SEQ ID NO:41. In some embodiments, the EphA2 binding moiety competes for binding to EphA2 with an scFv consisting of SEQ ID NO:41.

In one embodiment, the invention is a use of a liposome in a method of treating cancer in a human patient need thereof, the method comprising administering to the human patient a therapeutically effective amount of the liposome in a pharmaceutical composition. In some embodiments, the cancer is selected from the group consisting of solid tumors, breast, gastric, esophageal, lung, prostate and ovarian cancer. In some embodiments, the cancer is triple negative breast cancer (TNBC). In some embodiments, the cancer is gastric, gastroesophageal junction or esophageal carcimona. In some embodiments, the cancer is small cell lung cancer or non-small cell lung cancer. In some embodiments, the cancer is ovarian cancer, endometrial carcinoma or urothelial carcinoma. In some embodiments, the cancer is prostate adenocarcinoma. In some embodiments, the cancer is squamous cell carcinoma of the head and neck (SCCHN). In some embodiments, the cancer is Pancreatic ductal adenocarcinoma (PDAC). In some embodiments, the cancer is soft tissue sarcoma subtypes except GIST, desmoid tumors and pleomorphic rhabdomyosarcoma.

In some embodiments, the liposome comprises an EphA2 binding scFv moiety comprising the sequence of SEQ ID NO:41. In some embodiments, the liposome comprises the docetaxel prodrug selected from the group consisting of: Compound 1, Compound 2, Compound 3, Compound 4, Compound 5, and Compound 6. In some embodiments, the docetaxel prodrug is Compound 3. In some embodiments, the docetaxel prodrug is Compound 6,

In some embodiments, the hematologic toxicity is less than that of free docetaxel. In some embodiments, the hematologic toxicity of the dose of docetaxel delivered in the liposome is less than that of the same dose of free docetaxel.

EphA2-Targeted Liposomes for Delivery of Docetaxel

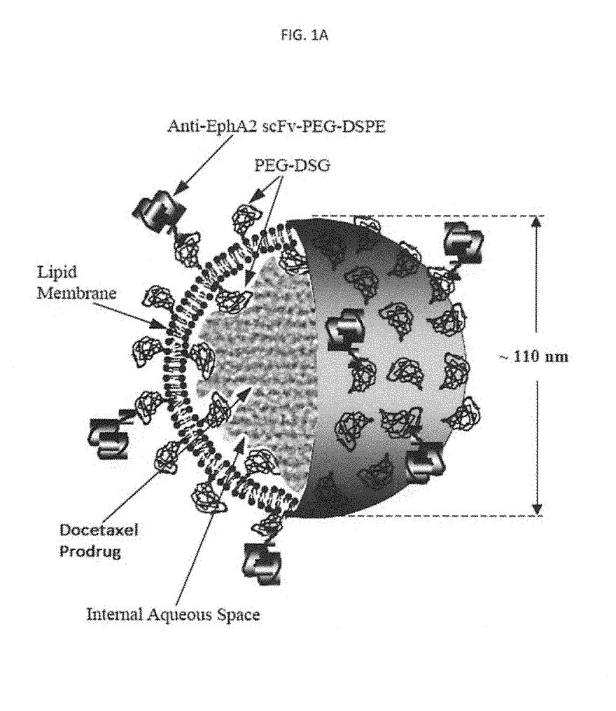

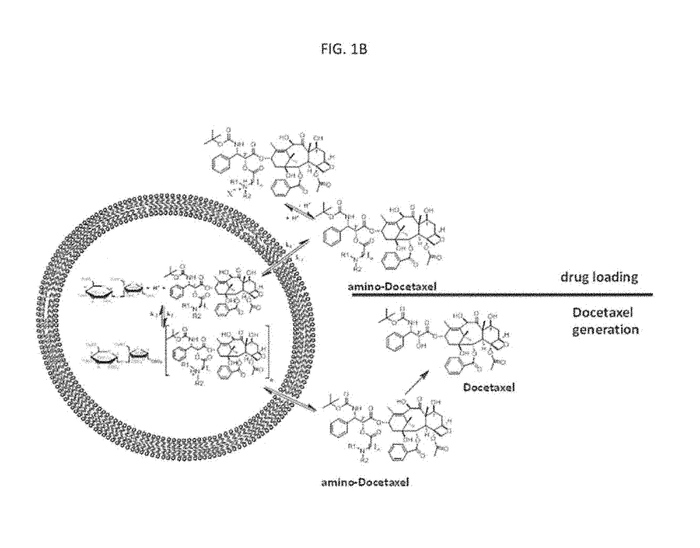

FIG. 1A is a schematic showing the structure of a PEGylated EphA2 targeted, nano-sized immunoliposome (nanoliposome) encapsulating a docetaxel prodrug (e.g., having a liposome size on the order of about 100 nm). The immunoliposome can include an Ephrin A2 (EphA2) targeted moiety, such as a scFv, bound to the liposome (e.g., through a covalently bound PEG-DSPE moiety). The PEGylated EphA2 targeted liposome encapsulating a docetaxel prodrug can be created by covalently conjugating single chain Fv (scFv) antibody fragments that recognize the EphA2 receptor to pegylated liposomes, containing docetaxel in the form of a prodrug described herein, resulting in an immunoliposomal drug product (FIG. 1A). In one particular example of a PEGylated EphA2 targeted liposome encapsulating a docetaxel prodrug (herein designated “EphA2-ILs-DTX”), the lipid membrane can be composed of egg sphingomyelin, cholesterol, and 1,2-distearoyl-sn-glyceryl methoxypolyethylene glycol ether (PEG-DSG). The nanoliposomes can be dispersed in an aqueous buffered solution, such as a sterile pharmaceutical composition formulated for parenteral administration to a human.

The EphA2 targeted nanoliposome of FIG. 1A is preferably a unilamellar lipid bilayer vesicle, approximately 110 nm in diameter, which encapsulates an aqueous space which contains a compound of disclosed herein in a gelated or precipitated state, as sucrosofate (sucrose octasulfate) salt. Example 1 describes methods of preparing a PEGylated EphA2 targeted liposome encapsulating a docetaxel prodrug.

The docetaxel prodrug can be stabilized in the liposomal interior during storage and while the intact liposome is in the general circulation, but is hydrolyzed rapidly (e.g., t½=˜10 h) to the active docetaxel upon release from the liposome and entering the environment of the circulating blood. FIG. 1B is a depiction of docetaxel nanogenerator with a docetaxel prodrug compound as disclosed herein. A docetaxel prodrug can be loaded at mildly acidic pH and entrapped in the acidic interior of liposomes, using an electrochemical gradient where it is stabilized in a non-soluble form. Upon release from the liposome, the docetaxel prodrug is subsequently converted to active docetaxel by simple base-mediated hydrolysis at neutral pH.

Docetaxel Prodrug Compounds

The PEGylated EphA2 targeted liposome encapsulating a docetaxel prodrug can encapsulate one or more suitable docetaxel prodrugs. Preferably, the docetaxel prodrug comprises a weak base such as tertiary amine introduced to the 2′ or 7 position hydroxyl group of docetaxel through ester bond to form a docetaxel prodrug. Preferred 2′-docetaxel prodrugs suitable for loading into a liposome are characterized by comparatively high stability at acidic pH but convert to docetaxel at physiological pH through enzyme-independent hydrolysis.

As shown in FIG. 1B, the chemical environment of the 2′-ester bond can be tuned systematically to obtain docetaxel prodrugs that are stable at relatively low pH but will release free docetaxel rapidly at physiologic pH through hydrolysis. Docetaxel prodrugs are loaded into liposome at relatively low pH by forming stable complexes with trapping agents such as polysulfated polyols, for example, sucrose octasulfate. The trapping agent sucrose octasulfate can be included in the liposome interior, as a solution of its amine salt, such as diethylamine salt (DEA-SOS), or triethylamine salt (TEA-SOS). The use of amine salts of the trapping agents helps to create a transmembrane ion gradient that aids the prodrug loading into the liposome and also to maintain the acidic intraliposomal environment favorable for keeping the prodrug from premature conversion to docetaxel before the prodrug-loaded liposome reaches its anatomical target. Encapsulation of docetaxel prodrugs inside liposome in such a way allows the practical application of pH triggered release of docetaxel upon release from the liposome within the body of a patient. Thus, the liposome that encapsulates docetaxel-prodrug can be called docetaxel nanogenerator.

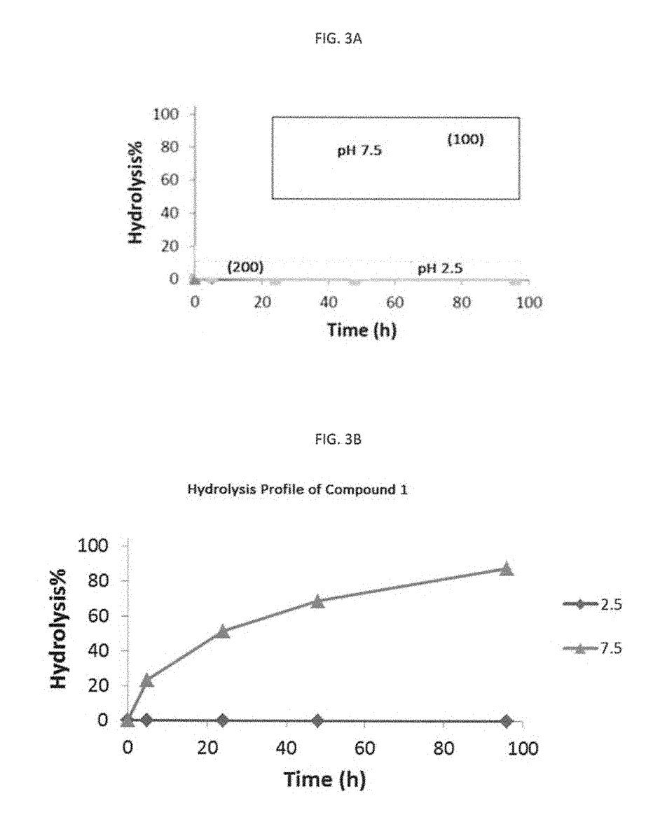

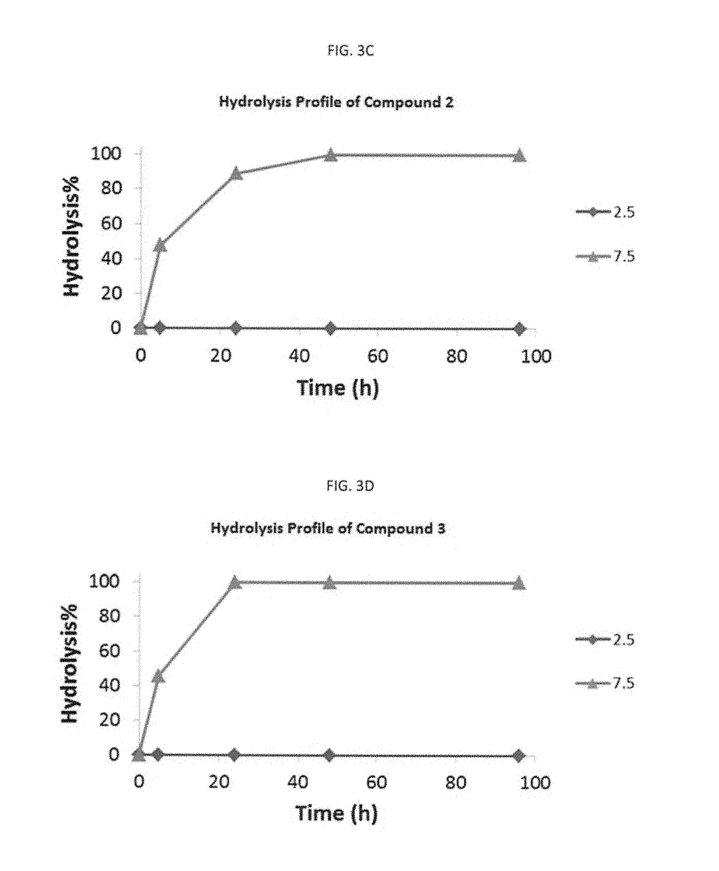

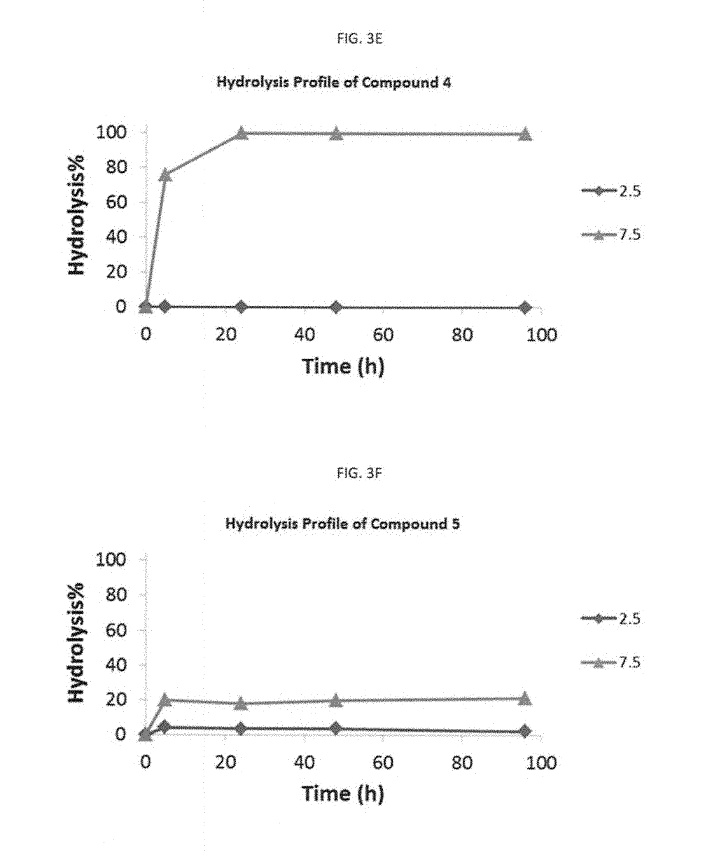

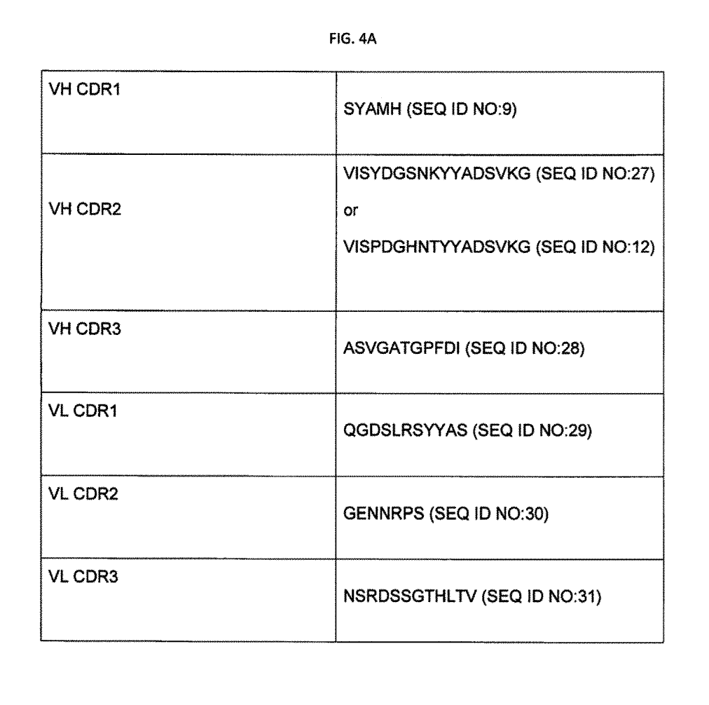

Suitable docetaxel prodrugs for liposome loading and encapsulation can be determined by evaluating the hydrolysis profile of docetaxel prodrug compounds. The hydrolysis profile can be obtained using the method of Example 11. Preferred docetaxel prodrugs for encapsulation in a liposome have a hydrolysis profile at 37° C. (e.g., in 20 mM HEPES buffer) that includes high stability (e.g., less than 10%, less than 5%, more preferably less than 1%, or no detectable ester hydrolysis yielding the activated drug after four or more days) at pH 2.5, but rapid and complete hydrolysis to form docetaxel (e.g., at least 70%, at least 80%, at least 90% or essentially complete ester hydrolysis to form the activated drug after 24 hours) at pH 7.5 (physiological pH). FIG. 3A illustrates the hydrolysis profile at 37 deg C for a docetaxel prodrug suitable for loading into a liposome, using the method of Example 11. At pH 2.5, the docetaxel prodrug undergoes minimal (preferably less than about 10%, more preferably less than about 5%) hydrolysis to form the docetaxel compound. The region (200) in FIG. 3A illustrates a preferred range of values for the hydrolysis % over time. At pH 7.5, the docetaxel prodrug preferably undergoes a high level of hydrolysis to form docetaxel (preferably at least about 50% after 24 hours, more preferably at least about 60% hydrolysis after 24 hours). In FIG. 3A, box (100) shows a range of preferred values for hydrolysis of a docetaxel prodrug at pH 7.5, using the method of Example 11. For example, a docetaxel prodrug profile at 37° C. in 20 mM HEPES buffer can include no detectable ester hydrolysis yielding the activated drug after four days at pH 2.5 and essentially complete ester hydrolysis to form the activated drug after 24 hours at pH 7.5. Compounds

Preferably, the docetaxel prodrug is a compound of formula (I), including pharmaceutically acceptable salts thereof, where R1, R2 and n are selected to provide desired liposome loading and stability properties, as well as desired docetaxel generation (e.g., as measured by the hydrolysis profile at various pH values, as disclosed herein). The docetaxel prodrug (DTX′) compounds can form a pharmaceutically acceptable salt within the liposome (e.g., a salt with a suitable trapping agent such as a sulfonated polyol). In some examples, the compounds of formula (I) where R1 and R2 are independently H or lower alkyl (preferably C₁-C₄ linear or branched alkyl, most preferably C₂ or C₃), and n is an integer (preferably 1-4, most preferably 2-3). Examples of docetaxel prodrugs also include docetaxel analog compounds having a 2′ substituents —O—(CO)—(CH₂)ₙN(R1)(R2) in formula (I) that are substituted in the manner disclosed in formula (III) of U.S. Pat. No. 4,960,790 to Stella et al. (filed Mar. 9, 1989), incorporated herein by reference in its entirety.

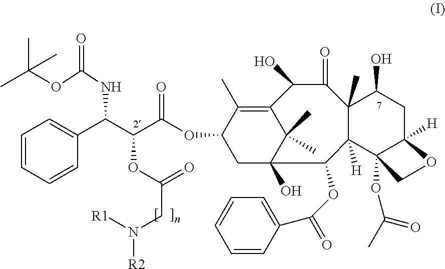

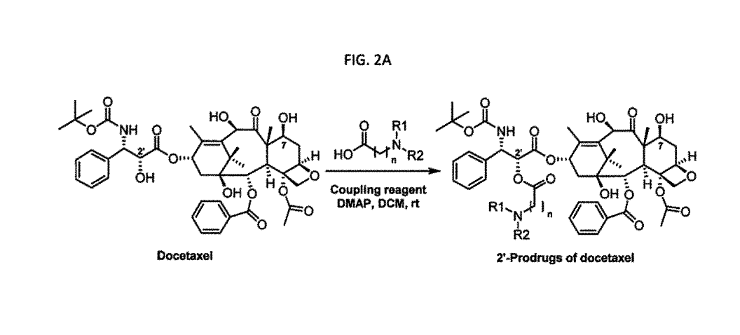

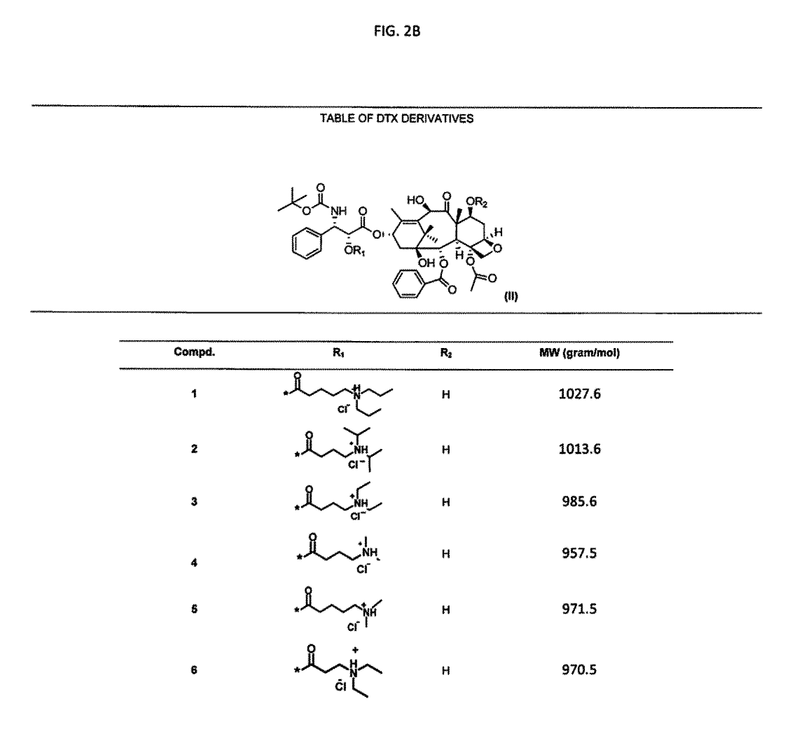

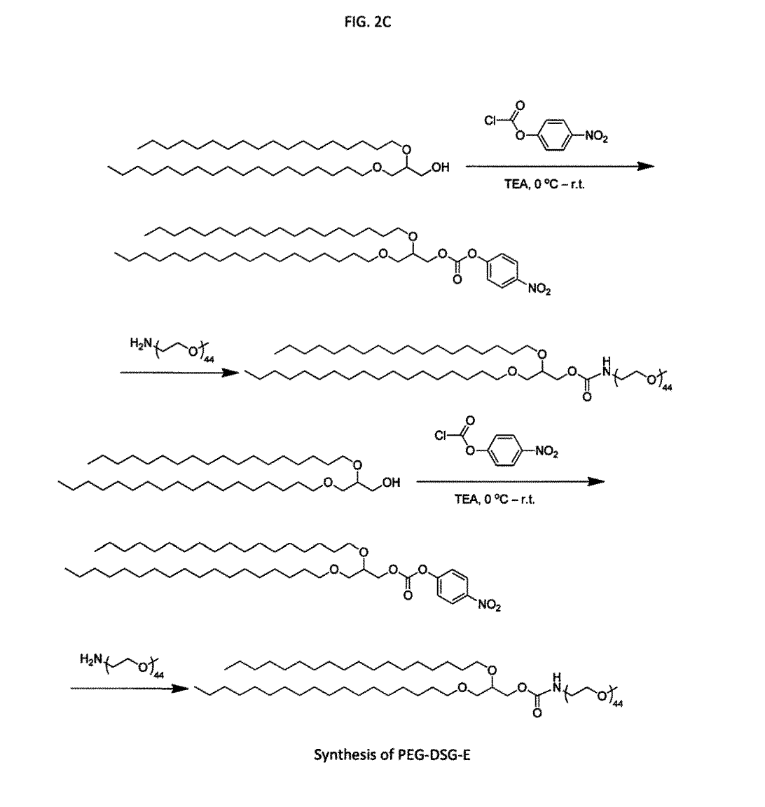

The docetaxel prodrugs, including compounds of Formula (I), can be prepared using the reaction Scheme in FIG. 2A. Two specific preparations of docetaxel prodrugs are described in Example 10A (Compound 3) and Example 10B (Compound 4). Other examples of docetaxel prodrugs include 2′-(2-(N,N′-diethylamino)propionyl)-docetaxel or 7-(2-(N,N′-diethylamino)propionyl)-docetaxel.

Preferred docetaxel prodrug compounds of formula (I) include compounds where (n) is 2 or 3, to provide a rapid hydrolysis rate at pH 7.5 and a sufficiently high relative hydrolysis rate for the compound at pH 7.5 compared to pH 2.5 (e.g., selecting docetaxel prodrugs with maximum hydrolysis rate of the docetaxel prodrug to docetaxel at pH 7.5 compared to the hydrolysis rate at pH 2.5, using the hydrolysis assay of Example 11) FIGS. 3C-3F show hydrolysis profiles for various examples of docetaxel prodrugs.

EphA2 Targeted scFv Moiety

The docetaxel-generating liposome can comprise a EphA2 targeting moiety. The targeting moiety can be a single chain Fv (“scFv”), a protein that can be covalently bound to a liposome to target the docetaxel-producing liposomes disclosed herein. The scFv can be comprised of a single polypeptide chain in which a VH and a VL are covalently linked to each other, typically via a linker peptide that allows the formation of a functional antigen binding site comprised of VH and VL CDRs. An Ig light or heavy chain variable region is composed of a plurality of “framework” regions (FR) alternating with three hypervariable regions, also called “complementarity determining regions” or “CDRs”. The extent of the framework regions and CDRs can be defined based on homology to sequences found in public databases. See, for example, “Sequences of Proteins of Immunological Interest,” E. Kabat et al., Sequences of proteins of immunological interest, 4th ed. U.S. Dept. Health and Human Services, Public Health Services, Bethesda, Md. (1987). All scFv sequence numbering used herein is as defined by Kabat et al.

As used herein, unless otherwise indicated, the term “anti-EphA2 scFv” refers to an scFv that immunospecifically binds to EphA2, preferably the ECD of EphA2. An EphA2-specific scFv does not immunospecifically bind to antigens not present in EphA2 protein.

In certain embodiments, an scFv disclosed herein includes one or any combination of VH FR1, VH FR2, VH FR3, VL FR1, VL FR2, and VL FR3 set forth in Table 1. In one embodiment, the scFv contains all of the frameworks of Table 1 below.

TABLE 1
Exemplary Framework Sequences

| | |
|---|---|
| VH FR1 | QVQLVQSGGGLVQPGGSLRLSCAASGFTFS |
| (SEQ ID NO: 1) | |
| VH FR2 | WVRQAPGKGLEWVT |
| (SEQ ID NO: 2) | |
| VH FR3 | RFTISRDNSKNTLYLQMNSLRAEDTAVYYCAR |
| (SEQ ID NO: 3) | |
| VH FR4 | WGQGTLVTVSS |
| (SEQ ID NO: 4) | |
| VL FR1 | SSELTQPPSVSVAPGQTVTITC |
| (SEQ ID NO: 5) | |
| VL FR2 | WYQQKPGTAPKLLIY |
| (SEQ ID NO: 6) | |
| VL FR3 | GVPDRFSGSSSGTSASLTITGAQAEDEADYYC |
| (SEQ ID NO: 7) | |
| VL FR4 | FGGGTKLTVLG |
| (SEQ ID NO: 8) | |

In certain aspects, an scFv disclosed herein is thermostable, e.g., such that the scFv is well-suited for robust and scalable manufacturing. As used herein, a “thermostable” scFv is an scFv having a melting temperature (Tm) of at least about 70° C., e.g., as measured using differential scanning fluorimetry (DSF).

A preferred anti-EphA2 scFv binds to the extracellular domain of EphA2 polypeptide, i.e., the part of the EphA2 protein spanning at least amino acid residues 25 to 534 of the sequence set forth in GenBank Accession No. NP 004422.2 or UniProt Accession No. P29317.

In certain embodiments, an anti-EphA2 scFv disclosed herein includes a VH CDR1, VH CDR2, VH CDR3, VL CDR1, VL CDR2, and VL CDR3 each with a sequence as set forth in Table 2. Note that the VH CDR2 sequence (also referred to as CDRH2) will be any one selected from the 18 different VH CDR2 sequences set forth in Table 2.

TABLE 2
Complementary Determining Regions (CDRs)

| | |
|---|---|
| VH CDR1 (SEQ ID NO: 9) | SYAMH |
| VH CDR2 (SEQ ID NO: 10) | VISPAGNNTYYADSVK |
| VH CDR2 (SEQ ID NO: 11) | VISPAGRNKYYADSVK |
| VH CDR2 (SEQ ID NO: 12) | VISPDGHNTYYADSVKG |
| VH CDR2 (SEQ ID NO: 13) | VISPHGRNKYYADSVK |
| VH CDR2 (SEQ ID NO: 14) | VISRRGDNKYYADSVK |
| VH CDR2 (SEQ ID NO: 15) | VISNNGHNKYYADSVK |
| VH CDR2 (SEQ ID NO: 16) | VISPAGPNTYYADSVK |
| VH CDR2 (SEQ ID NO: 17) | VISPSGHNTYYADSVK |
| VH CDR2 (SEQ ID NO: 18) | VISPNGHNTYYADSVK |
| VH CDR2 (SEQ ID NO: 19) | AISPPGHNTYYADSVK |
| VH CDR2 (SEQ ID NO: 20) | VISPTGANTYYADSVK |
| VH CDR2 (SEQ ID NO: 21) | VISPHGSNKYYADSVK |
| VH CDR2 (SEQ ID NO: 22) | VISNNGHNTYYADSVK |
| VH CDR2 (SEQ ID NO: 23) | VISPAGTNTYYADSVK |
| VH CDR2 (SEQ ID NO: 24) | VISPPGHNTYYADSVK |
| VH CDR2 (SEQ ID NO: 25) | VISHDGTNTYYADSVK |
| VH CDR2 (SEQ ID NO: 26) | VISRHGNNKYYADSVK |
| VH CDR2 (SEQ ID NO: 27) | VISYDGSNKYYADSVKG |
| VH CDR3 (SEQ ID NO: 28) | ASVGATGPFDI |
| VL CDR1 (SEQ ID NO: 29) | QGDSLRSYYAS |
| VL CDR2 (SEQ ID NO: 30) | GENNRPS |
| VL CDR3 (SEQ ID NO: 31) | NSRDSSGTHLTV |

In certain embodiments, an scFv disclosed herein is an internalizing anti-EphA2 scFv. Binding of such an scFv to the ECD of and EphA2 molecule present on the surface of a living cell under appropriate conditions results in internalization of the scFv. Internalization results in the transport of an scFv contacted with the exterior of the cell membrane into the cell-membrane-bound interior of the cell. Internalizing scFvs find use, e.g., as vehicles for targeted delivery of drugs, toxins, enzymes, nanoparticles (e.g., liposomes), DNA, etc., e.g., for therapeutic applications.

Certain scFvs described herein are single chain Fv scFvs e.g., scFvs or (scFv′)2s. In such scFvs, the VH and VL polypeptides are joined to each other in either of two orientations (i.e., the VH N-terminal to the VL, or the VL N-terminal to the VH) either directly or via an amino acid linker. Such a linker may be, e.g., from 1 to 50, 5 to 40, 10 to 30, or 15 to 25 amino acids in length. In certain embodiments, 80% or greater, 85% or greater, 90% or greater, 95% or greater, or 100% of the residues of the amino acid linker are serine (S) and/or glycine (G). Suitable exemplary scFv linkers comprise or consist of the sequence:

| | |
|---|---|
| ASTGGGGSGGGGSGGGGSGGGGS, | (SEQ ID NO: 32) |
| GGGGSGGGGSGGGGSGGGGS, | (SEQ ID NO: 33) |
| GGGGSGGGGSGGGGS, | (SEQ ID NO: 34) |
| ASTGGGGAGGGGAGGGGAGGGGA, | (SEQ ID NO: 35) |
| GGGGAGGGGAGGGGAGGGGA, | (SEQ ID NO: 36) |
| TPSHNSHQVPSAGGPTANSGTSGS, | (SEQ ID NO: 37) |
| and | |
| GGSSRSSSSGGGGSGGGG. | (SEQ ID NO: 38) |

An exemplary internalizing anti-EphA2 scFv is scFv TS1 (SEQ ID NO:40). In scFv TS1, and in certain other scFvs disclosed herein, the VH of the scFv is at the amino terminus of the scFv and is linked to the VL by a linker indicated in italics. The CDRs of the scFvs are underlined and are presented in the following order: VH CDR1, VH CDR2, VH CDR3, VL CDR1, VL CDR2, and VL CDR3.

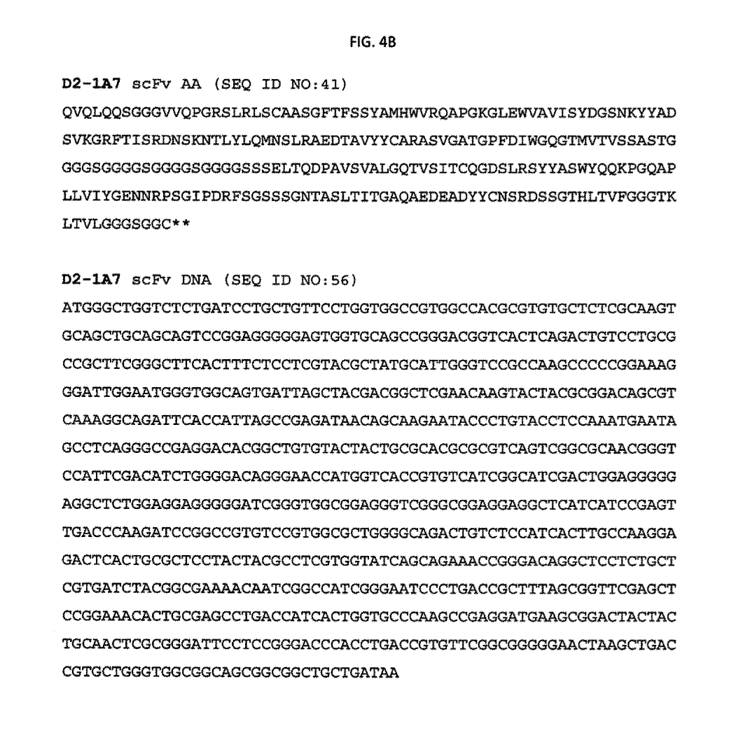

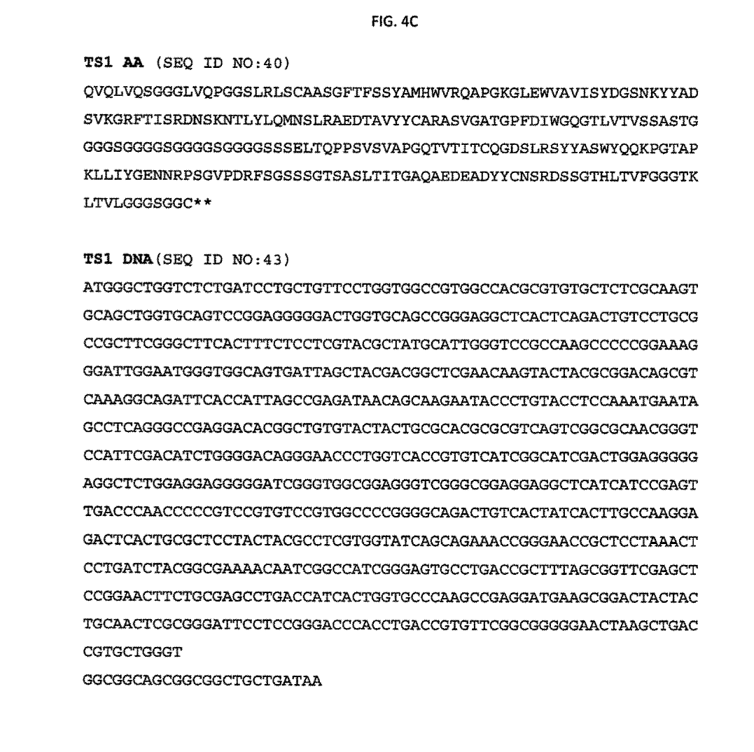

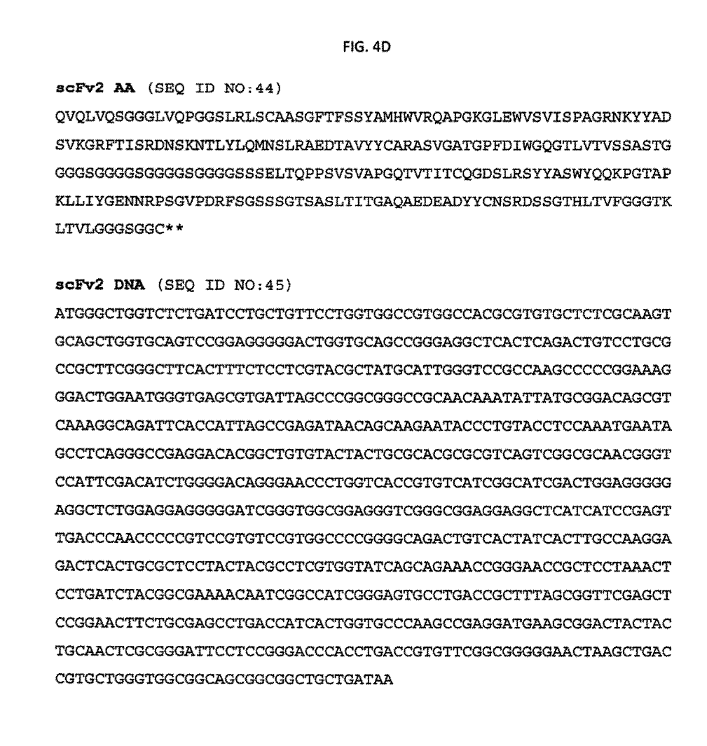

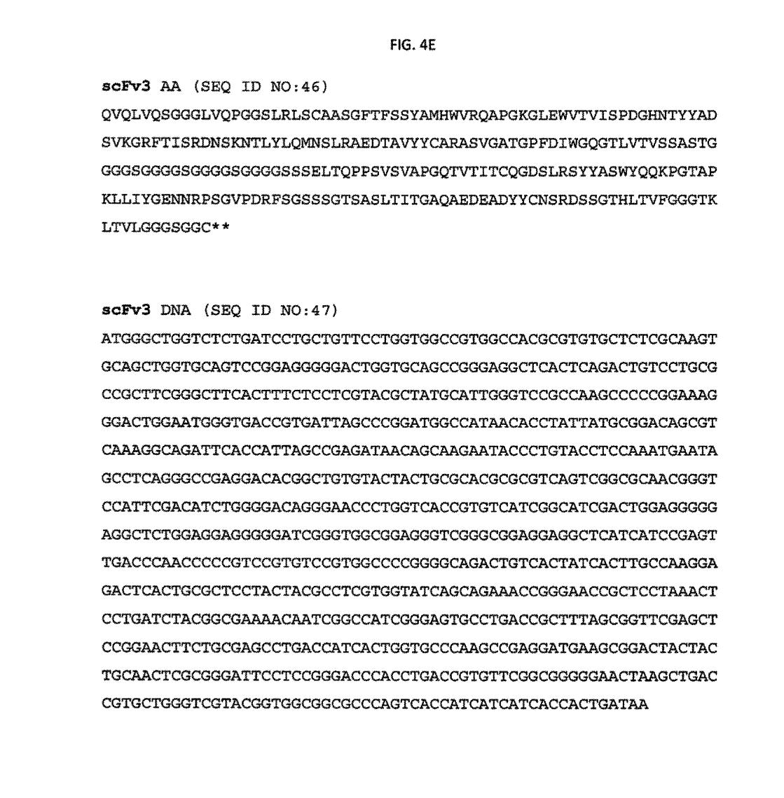

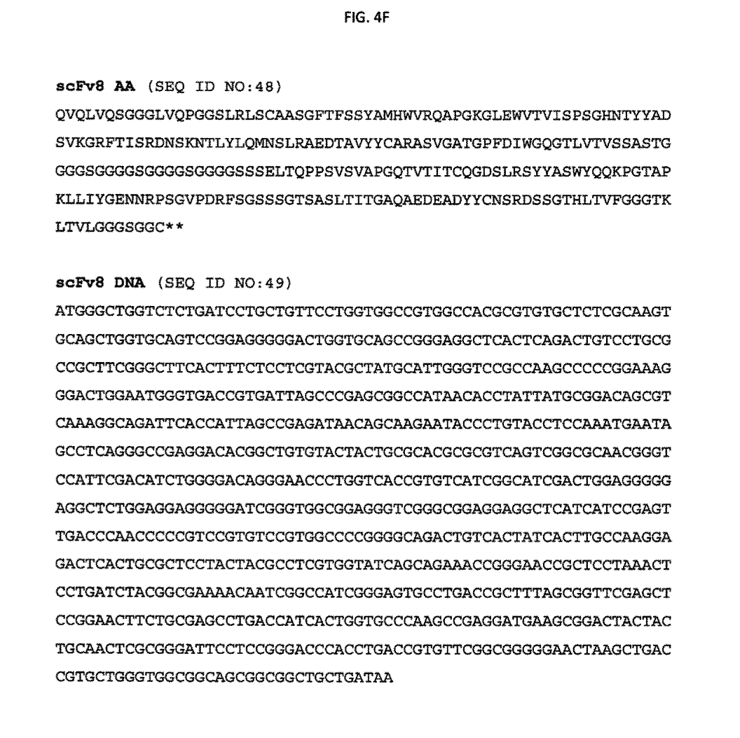

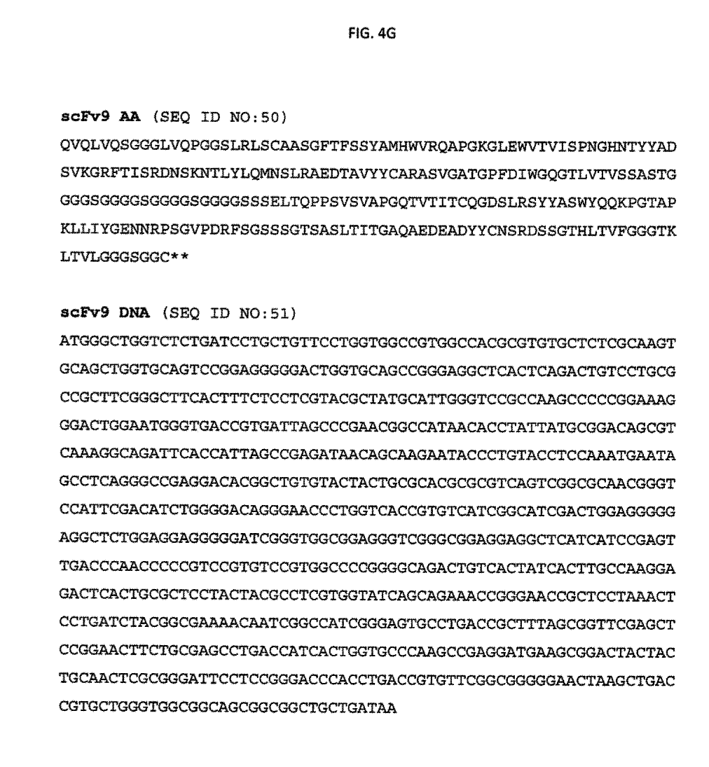

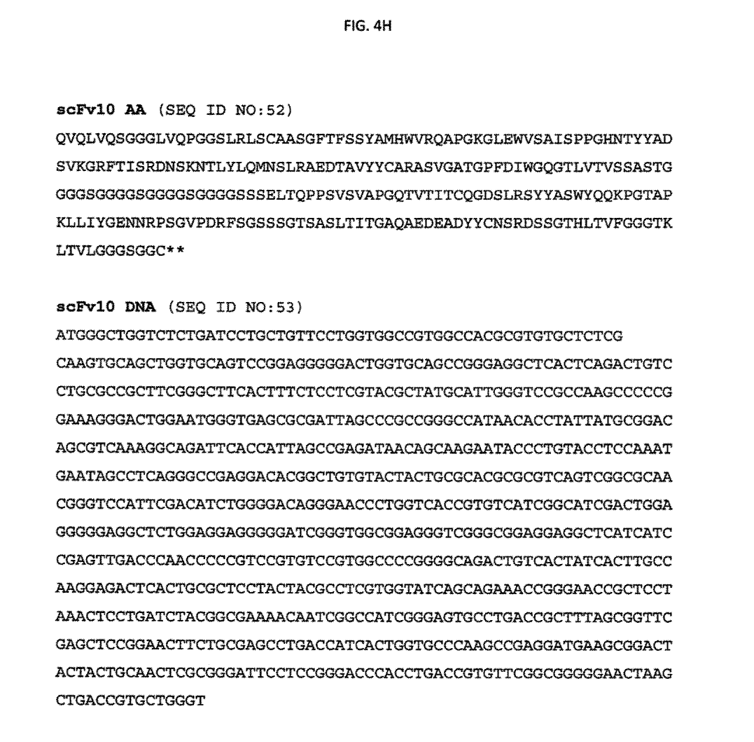

The docetaxel-generating EphA2-targeted liposomes can also include one or more EphA2 targeted scFv sequences shown FIG. 4B (SEQ ID NO:41, designated “D2-1A7”, encoded by the DNA sequence of SEQ ID NO:56 designated “D2-1A7 DNA”), or FIG. 4C (SEQ ID NO:40, designated “TS1”, encoded by the DNA sequence of SEQ ID NO:43 designated “TS1 DNA”), or FIG. 4D (SEQ ID NO:44, designated “scFv2”, encoded by the DNA sequence of SEQ ID NO:45 designated “scFv2 DNA”), or FIG. 4E (SEQ ID NO:46, designated “scFv3”, encoded by the DNA sequence of SEQ ID NO:47 designated “scFv3 DNA”), or FIG. 4F (SEQ ID NO:48, designated “scFv8”, encoded by the DNA sequence of SEQ ID NO:49 designated “scFv8 DNA”), or FIG. 4G (SEQ ID NO:50, designated “scFv9”, encoded by the DNA sequence of SEQ ID NO:51 designated “scFv9 DNA”) or FIG. 4H (SEQ ID NO:52, designated “scFv10”, encoded by the DNA sequence of SEQ ID NO:53 designated “scFv10 DNA”) or FIG. 4I (SEQ ID NO:54, designated “scFv13”, encoded by the DNA sequence of SEQ ID NO:55 designated “scFv13 DNA”).

Also provided are variants of scFv TS1 in which VH CDR2 is selected from any of the 18 different CDRH2 sequences set forth above in Table 2.

Using the information provided herein, the scFvs disclosed herein may be prepared using standard techniques. For example, the amino acid sequences provided herein can be used to determine appropriate nucleic acid sequences encoding the scFvs and the nucleic acids sequences then used to express one or more of the scFvs. The nucleic acid sequence(s) can be optimized to reflect particular codon “preferences” for various expression systems according to standard methods.

Using the sequence information provided herein, the nucleic acids may be synthesized according to a number of standard methods. Oligonucleotide synthesis, is conveniently carried out on commercially available solid phase oligonucleotide synthesis machines or manually synthesized using, for example, the solid phase phosphoramidite triester method. Once a nucleic acid encoding an scFv disclosed herein is synthesized, it can be amplified and/or cloned according to standard methods.

Expression of natural or synthetic nucleic acids encoding the scFvs disclosed herein can be achieved by operably linking a nucleic acid encoding the scFv to a promoter (which may be constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the scFv. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which contain a strong promoter to direct transcription, a ribosome binding site for translational initiation, and a transcription/translation terminator, each in functional orientation to each other and to the protein-encoding sequence. The scFv gene(s) may also be subcloned into an expression vector that allows for the addition of a tag sequence, e.g., FLAGT™ or His6, at the C-terminal end or the N-terminal end of the scFv (e.g. scFv) to facilitate identification, purification and manipulation. Once the nucleic acid encoding the scFv is isolated and cloned, one can express the nucleic acid in a variety of recombinantly engineered cells. Examples of such cells include bacteria, yeast, filamentous fungi, insect, and mammalian cells.

Isolation and purification of an scFv disclosed herein can be accomplished by isolation from a lysate of cells genetically modified to express the protein constitutively and/or upon induction, or from a synthetic reaction mixture, with purification, e.g., by affinity chromatography (e.g., using Protein A or Protein G). The isolated scFv can be further purified by dialysis and other methods normally employed in protein purification.

The present disclosure also provides cells that produce subject scFvs. For example, the present disclosure provides a recombinant host cell that is genetically modified with one or more nucleic acids comprising nucleotide sequence encoding an scFv disclosed herein. DNA is cloned into, e.g., a bacterial (e.g., bacteriophage), yeast (e.g. Saccharomyces or Pichia) insect (e.g., baculovirus) or mammalian expression system. One suitable technique uses a filamentous bacteriophage vector system. See, e.g., U.S. Pat. No. 5,885,793; U.S. Pat. No. 5,969,108; and U.S. Pat. No. 6,512,097.

Additional examples of EphA2 targeting sequences for the EphA2-targeted nanoliposome include sequences disclosed in Zhou et al in U.S. Pat. No 9,220,772, incorporated by reference. Zhou et al. discloses an isolated monoclonal antibody that specifically binds an epitope of EphA2, wherein the epitope is specifically bound by an antibody comprising: a variable heavy chain (VH) polypeptide comprising a VH CDR1 comprising the amino acid sequence SYAMH (SEQ ID NO:9), a VH CDR2 comprising the amino acid sequence VISYDGSNKYYADSVKG (SEQ ID NO: 27), and a VH CDR3 comprising the amino acid sequence ASVGATGPFDI (SEQ ID NO: 28); and a variable light chain (VL) polypeptide comprising a VL CDR1 comprising the amino acid sequence QGDSLRSYYAS (SEQ ID NO:29), a VL CDR2 comprising the amino acid sequence GENNRPS (SEQ ID NO:30), and a VL CDR3 comprising the amino acid sequence NSRDSSGTHLTV (SEQ ID NO: 31).

The EphA2 Targeted scFv Amino Acid Sequence can be attached to the liposome using an EphA2 (scFv) to maleimide-activated PEG-DSPE. For example, the scFv-PEG-DSPE drug substance can be a fully humanized single chain antibody fragment (scFv) conjugated to maleimide PEG-DSPE via the C-terminal cysteine residue of scFv. In some examples, the EphA2 targeted scFv is conjugated covalently through a stable thioether bond to a lipopolymer lipid, Mal-PEG-DSPE, which interacts to form a micellular structure. Preferably, the scFv is not glycosylated.

Preparing EphA2-Targeted Liposomes for Delivery of Docetaxel

The docetaxel prodrug can be loaded into liposomes through different approaches. Remote loading methods enables high loading efficiency and good scalability. Typically, liposomes are prepared in a loading aid (trapping agent) that may include a gradient-forming ion and a drug-precipitating or drug-complexing agent. The extraliposomal loading aid is removed, e.g., by diafiltration to generate an ion gradient across the liposome bilayer. Selected drug can cross the lipid bilayer, accumulate inside the liposome at the expense of the ion gradient and form complexes or precipitates with the loading aid. If the liposome lipid is in the gel state at ambient temperature, the loading is effected at elevated temperatures where the liposome membrane is in the liquid crystalline state. When drug loading is complete, liposomes are rapidly chilled so that loaded drug can be retained within the rigid membrane. Any factor involved in the drug loading step may impact the loading efficiency.

The EphA2 targeted nano-liposome can be obtained by combining the Eph-A2 binding scFv with DSPE-PEG-Mal under conditions effective to conjugate the scFv to the DSPE-PEG-Mal moiety. The DSPE-PEG-Mal conjugate can be combined with a polysulfated polyol loading aid and other lipid components to form a liposome containing the polysulfated polyol encapsulated with a lipid vesicle.

Referring again to FIG. 1B, the drug can be loaded into a liposome encapsulating a trapping agent. The drug release rate can be controlled by varying the type and concentration of the trapping agents, as can the stability towards hydrolysis of the prodrug. Examples of trapping agents include but are not limited to ammonium sucroseoctasulfate (SOS), diethylammonium SOS (DEA-SOS), triethylammonium SOS (TEA-SOS), and diethylammonium dextran sulfate. The concentration of the trapping agent can be selected to provide desired drug loading properties, and can vary from 250 mN to 2 N depending on the drug to lipid ratio desired. Normality (N) of the trapping agent solution depends on the valency of its drug-complexing counter-ion and is a product of the counter-ion molarity and its valency. For example, the normality of DEA-SOS solution, SOS being an octavalent ion, is equal to SOS molar concentration times eight. Thus, 1 N SOS is equal to 0.125 M SOS. When DEA-SOS is used as the trapping agent, the concentration ranges preferably from 0.5 N to 1.5 N, most preferably from 0.85 N to 1.2 N A formulation employing TEA-SOS at 1.1 N can result in a final formulation containing 300-800 grams of docetaxel equivalent prodrug per mol of phospholipid. This results in a dose of lipid that is between 8 and 22 mg total lipid/kg (302-806 mg/m²) to patients at a dose of 250 mg docetaxel equivalents/m². The final formulation has a preferable drug-to-phospholipid ratio of 250-400 g docetaxel equivalents/mol phospholipid

Docetaxel prodrugs can be dissolved in either acidic buffer directly, or in the presence of other solubilizing reagents such as hexa(ethylene glycol) (PEG6) or poly(ethylene glycol) with the average molecular weight of 200, 300, or 400 (PEG-200, PEG-300, PEG-400). Under any circumstance, basic conditions should be avoided in the solubilization process for docetaxel prodrugs that hydrolyze under basic conditions.

Liposomes used for loading taxane prodrugs are prepared, e.g., by ethanol injection hydration and membrane extrusion methods. The lipid components can be selected to provide desired properties.

In general, a variety of lipid components can be used to make the liposomes. Lipid components usually include, but are not limited to (1) uncharged lipid components, e.g., cholesterol, ceramide, diacylglycerol, acylpoly(ethers) or alkylpoly(ethers) and (2) neutral phospholipids, e.g., diacylphosphatidyicholines, dialkylphosphatidylcholines, sphingomyelins, and diacylphosphatidylethanolamines. Various lipid components can be selected to fulfill, modify or impart one or more desired functions. For example, phospholipid can be used as principal vesicle-forming lipid. Inclusion of cholesterol is useful for maintaining membrane rigidity and decreasing drug leakage. Polymer-conjugated lipids can be used in the liposomal formulation to increase the lifetime of circulation via reducing liposome clearance by liver and spleen, or to improve the stability of liposomes against aggregation during storage, in the absence of circulation extending effect.

Preferably, the liposome comprises an uncharged lipid component, a neutral phospholipid component and a polyethylene (PEG)-lipid component. A preferred PEGylated lipid component is PEG(Mol. weight 2,000)-distearoylglycerol (PEG-DSG) or N-palmitoyl-sphingosine-1-{succinyl[methoxy(polyethylene glycol)2000]} (PEG-ceramide). For example, the lipid components can include egg sphingomyelin, cholesterol, PEG-DSG at a suitable molar ratio (e.g., comprising sphingomyelin and cholesterol at a 3:2 molar ratio with a desired amount of PEG-DSG). The amount of PEG-DSG is preferably incorporated in the amount of 10 mol % (e.g., 4-10 mol %) of the total liposome phospholipid, or less, such as, less than 8 mol % of the total phospholipid, and preferably between 5-7 mol % of the total phospholipid. In another embodiment, a sphingomyelin (SM) liposome is employed in the formulation which is comprised of sphingomyelin, cholesterol, and PEG-DSG-E at given mole ratio such as 3:2:0.03. The neutral phospholipid and PEG-lipid components used in this formulation are generally more stable and resistant to acid hydrolysis. Sphingomyelin and dialkylphosphatidylcholine are examples of preferred phospholipid components. More specifically, phospholipids with a phase transition temperature (Tm) greater than 37° C. are preferred. These include, but are not limited to, egg-derived sphingomyelin, 1,2-di-O-octadecyl-sn-glycero-3-phosphocholine, N-stearoyl-D-erythro-sphingosylphosphorylcholine, and N-palmitoyl-D-erythro-sphingosylphosphorylcholine. The choice of liposome formulation depends on the stability of specific prodrug under certain conditions and the cost of manufacturing.

Taxane prodrugs are loaded into liposomes at acidic pH ranging preferably from 4 to 6 in the presence of buffers preferably 5-40 mM. Suitable acidic buffers include but not limited to, 2-(N-morpholino)ethanesulfonic acid (MES), oxalic acid, succinic acid, manolic acid, glutaric acid, fumaric acid, citric acid, isocitric acid, aconitic acid, and propane-1,2,3-tricarboxylic acid. Different drug loading methods have been developed to facilitate efficient loading of taxane prodrugs into liposome. In one embodiment, prodrug solution is mixed with the liposome at room temperature first, followed by the pH adjustment and incubation at elevated temperature. In another embodiment, the pH of the prodrug solution and liposomes are adjusted first to desired loading pH, pre-warmed to the desired loading temperature, then mixed and incubated. In still another embodiment, prodrug is solubilized in 80% PEG6 solution at high concentration first, and added portion by portion into the pre-warmed liposome. In further embodiments, prodrugs are dissolved in 80% PEG400 first, diluted to about 8% PEG400 in dextrose MES buffer, mixed with liposome at room temperature first, then warmed up to the loading temperature.

Unencapsulated polysulfated polyol material can be removed from the composition. Then, the liposome containing the polysulfated polyol loading aid (preferably TEA-SOS or DEA SOS) can be contacted with the a suitable taxane or taxane prodrug, such as a docetaxel prodrug of Formula (I), preferably a docetaxel prodrug of Compound 3, Compound 4 or Compound 6, under conditions effective to load taxane or taxane prodrug into the liposome, preferably forming a stable salt with the encapsulated polysulfated polyol within the liposome. Simultaneously, the loading aid counter ion (e.g., TEA or DEA) leaves the liposome as the drug is loaded into the liposome. Finally, unencapsulated drug (e.g., docetaxel prodrug) is removed from the composition comprising the liposome. Methods of liposome drug loading are described in U.S. Pat. No. 8,147,867, filed May 2, 2005, incorporated by reference.

Examples of methods suitable for making liposome compositions include extrusion, reverse phase evaporation, sonication, solvent (e.g., ethanol) injection, microfluidization, detergent dialysis, ether injection, and dehydration/rehydration. The size of liposomes can be controlled by controlling the pore size of membranes used for low pressure extrusions or the pressure and number of passes utilized in microfluidization or any other suitable methods. In one embodiment, the desired lipids are first hydrated by thin-film hydration or by ethanol injection and subsequently sized by extrusion through membranes of a defined pore size; most commonly 0.05 μm, 0.08 μm, or 0.1 μm. Preferably, the liposomes have an average diameter of about 90-120 nm, more preferably about 110 nm.

### EXAMPLES

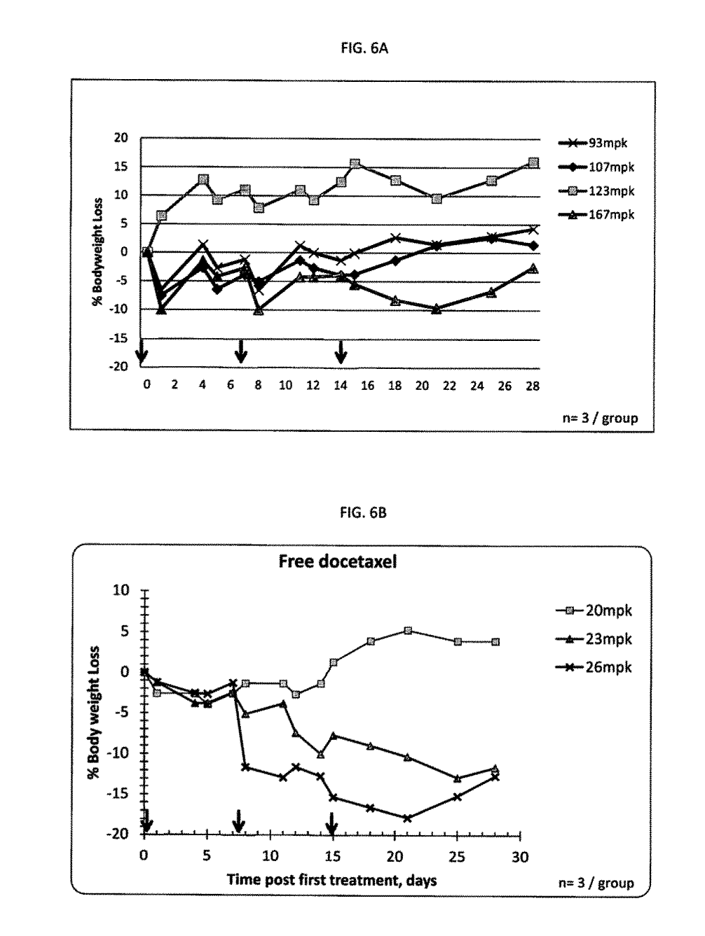

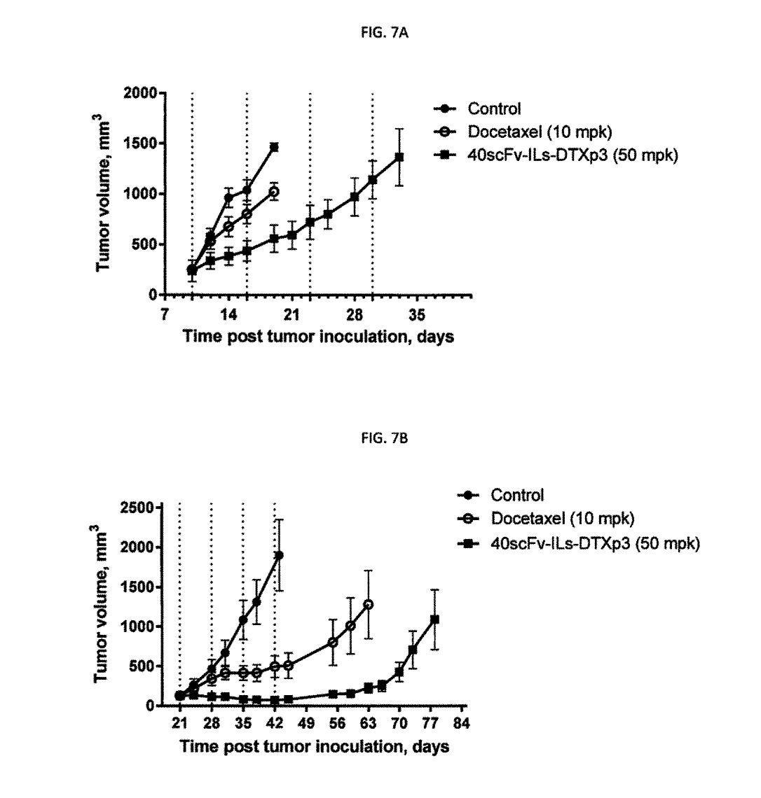

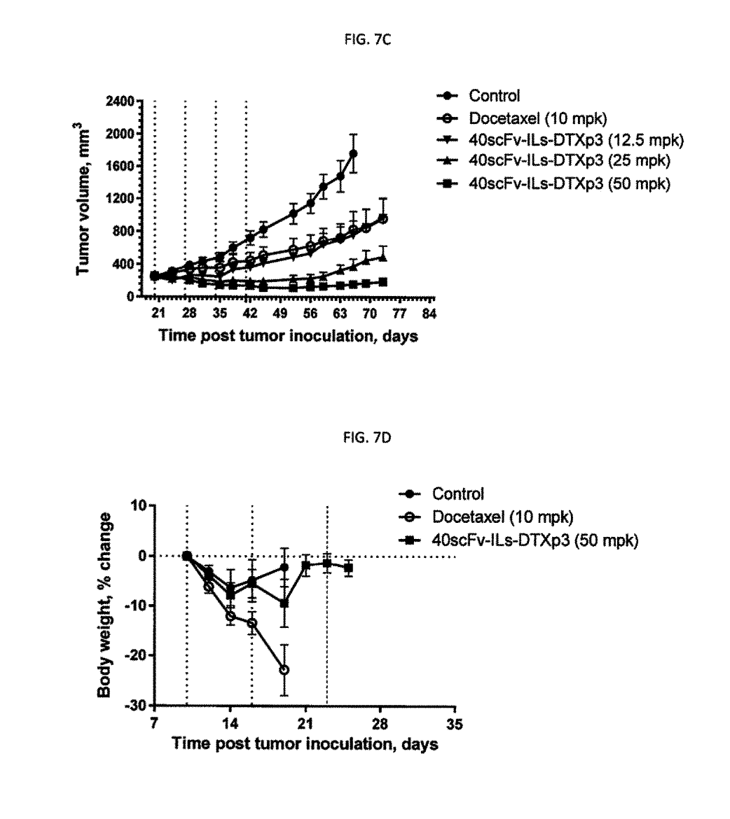

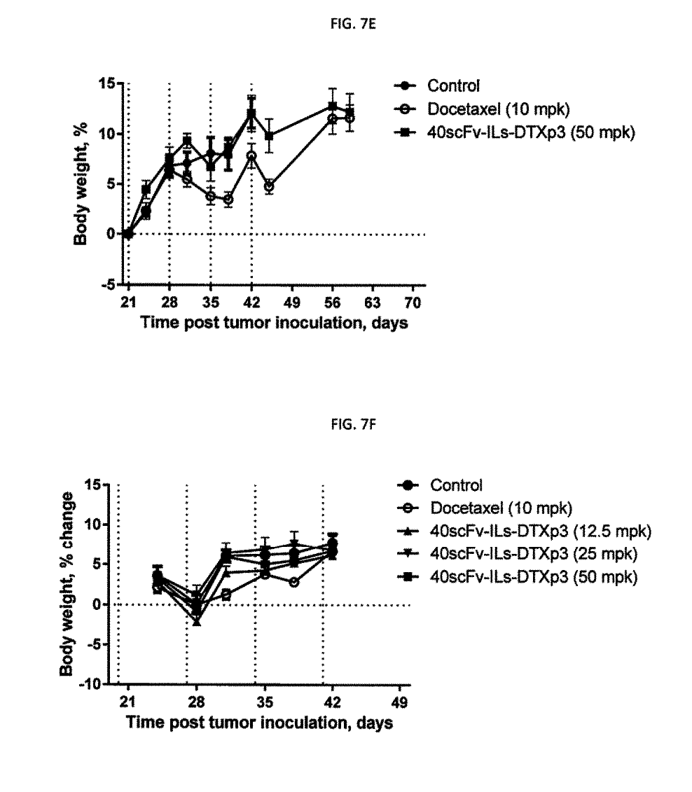

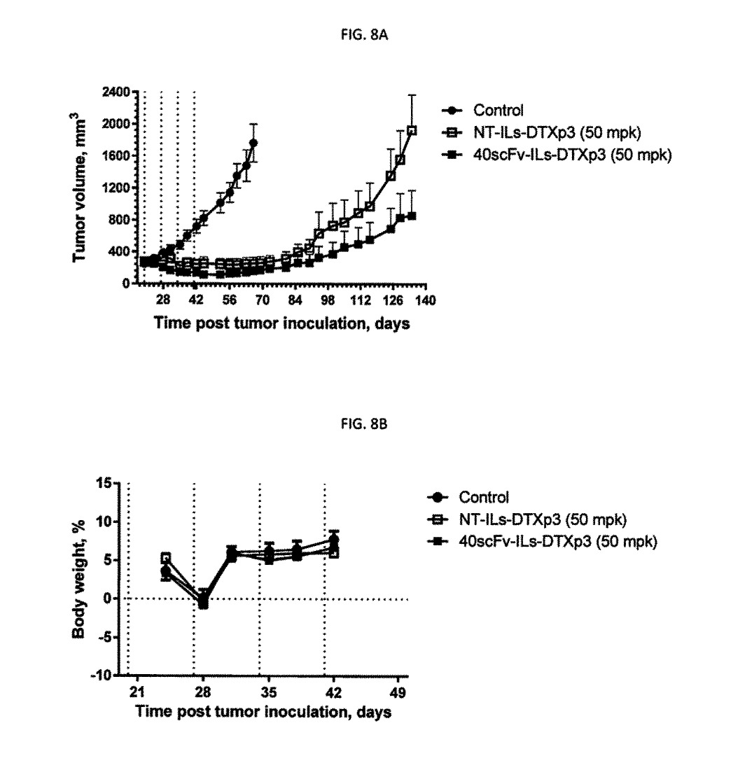

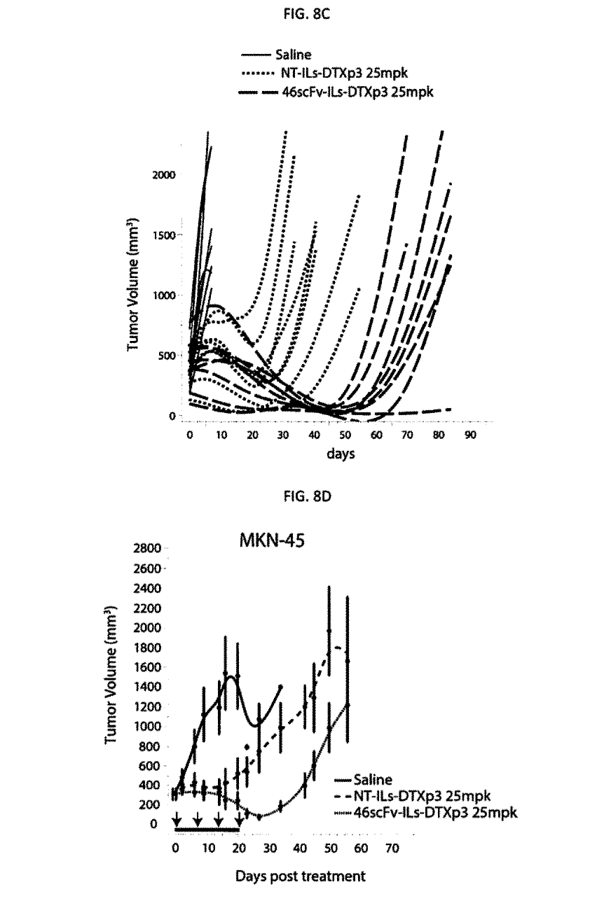

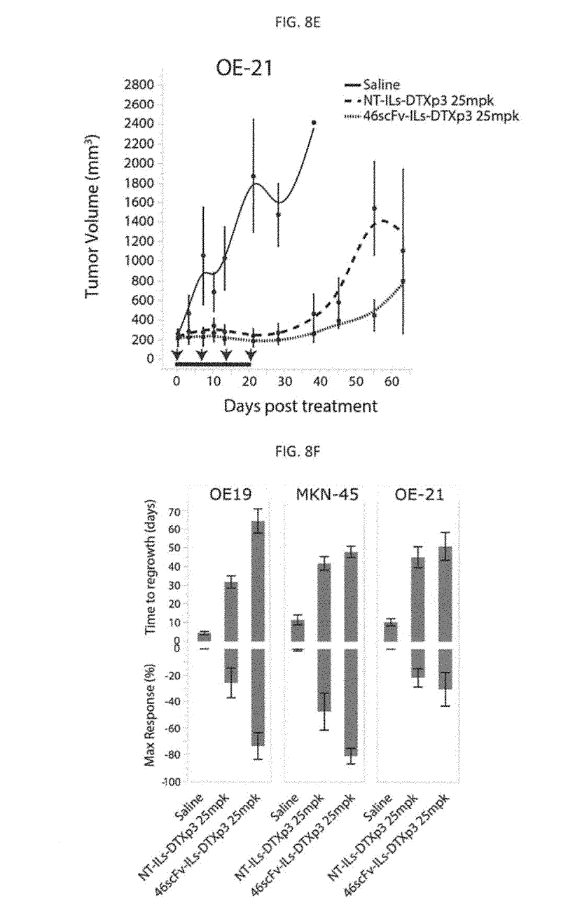

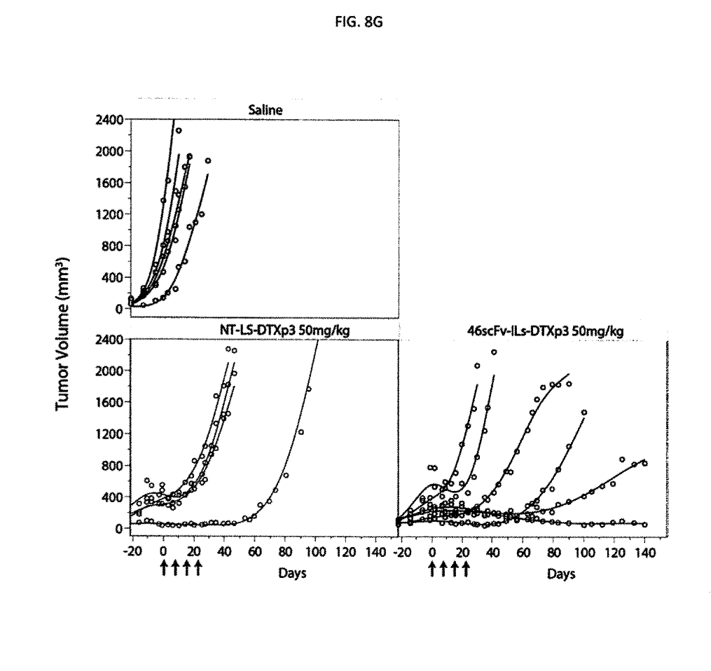

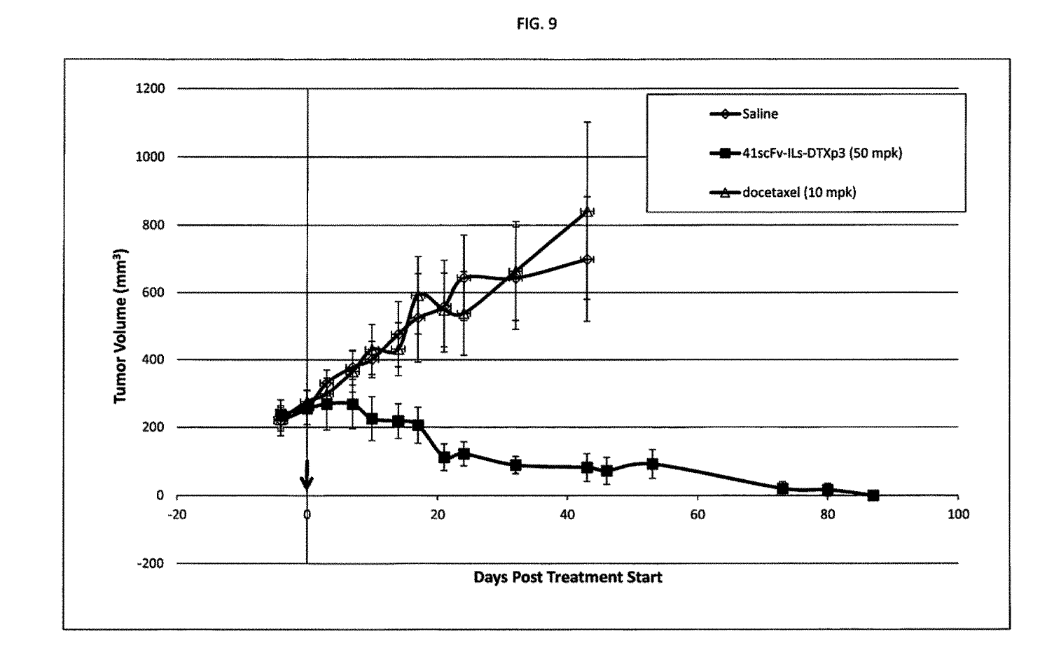

Unless otherwise indicated, an exemplary EphA2 targeted docetaxel-generating nanoliposome composition designated “EphA2-Ls-DTX” was tested as described in the examples below. Specifically (unless otherwise indicated) the experiments in the Examples below were obtained with a representative example of an EphA2-Ls-DTX targeted liposome designated 46scFv-ILs-DTXp3. 46scFv-ILs-DTXp3 comprises the compound of Formula (I) designated Compound 3 herein, encapsulated in a lipid vesicle formed from egg sphingomyelin, cholesterol and PEG-DSG in a weight ratio of about 4.4:1.6:1, and also includes a scFv moiety of HQ ID NO:46 covalently bound to PEG-DSPE in a weight ratio of between about 1:35 and 1:285 (e.g., between 1:36 and 1:284) of the total amount of phospholipid in the lipid vesicle, or preferably of about 1:142. In some examples (FIGS. 7A, 7B, 7C, 7D, 7E, 7F, 8A and 8B, and the corresponding Examples herein), the 40scFv-ILs-DTXp3 EphA2-targeted docetaxel-generating immunoliposome was used to obtain the data (comprising the same liposome composition as 46scFv-ILs-DTXp3 except for a difference in % PEG-DSG and drug to lipid ratio—Information about the formulation is added in the example description). In some examples (FIGS. 6A, 6B and 9) the 41scFv-ILs-DTXp3 EphA2-targeted docetaxel-generating immunoliposome was used to obtain the data (comprising the same liposome composition as 46scFv-ILs-DTXp3 except for a difference in % PEG-DSG and drug to lipid ratio—Information about the formulation is added in the example description). In some examples, the mol ratio of the scFv to the PEG-DSPE in the micelle is about 1:4. In some examples, the molecular weight of the scFv is about 26 kDa, and the molecular weight of the PEG-DSPE can be about 2.8 kDa. In some examples, the weight ratio of the scFv and total PEG-DSPE is between about 2:1 and 3:1, including a ratio of between about 2.25-2.5:1. The EphA2-Ls-DTX liposome can be formulated in a suitable composition to form a drug product, including a buffer system (e.g., citric acid and sodium citrate), an isotonicity agent (e.g., sodium chloride) and a sterile water vehicle as a diluent (e.g., water for injection). While certain embodiments of the invention are disclosed herein, these representative examples enable the preparation and use of variations of this disclosure.

### Example 1: Preparing EphA2 Targeted Docetaxel Generating Liposomes

### Example 1A Preparation of Sucrose Octasulfate Diethylamine Salt (DEA-SOS)

Step 1 packing and conditioning: Dowex 50Wx8-200 column. Load 350 g Dowex 50WX8-200 anion exchange resin in a large column (50 mm×300 mm), wash the resin with 1200 ml 1M sodium hydroxide, 1600 ml deionized water, 1200 ml 3 M hydrochloric acid, 1600 ml deionized water consecutively.

Step 2 sucrose octasulfate (SOS) solution: dissolve 30.0 g sodium sucrose octasulfate in 15 ml deionized water in a 50-ml centrifuge tube at 50 Celsius with vigorous vortex. The solution is syringe filtered through 0.2 μm membrane.

Step 3 load SOS solution on the Dowex column prepared in step 1. Elute the column with deionized water. Collect fractions having conductivity 50˜100 mS/cm as pool A, and larger than 100 mS/cm as pool B. Immediately titrate SOS in pool B with diethylamine to a final pH of 6.7˜7.1. In case that pH of pool B pasts pH 7.1, lower the pH using the acidic SOS from pool A. SOS concentration is determined by sulfate assay and verified by the titration data.

### Example 1B Preparation of PEG-DSG-E

PEG-DSG-E is a novel conjugate of ether lipid and polyethylene glycol (PEG) designed to be less labile to the hydrolysis conditions exposed to liposomes. Due to the use of carbamate linker and ether lipid, PEG-DSG-E is more stable under mild acidic condition and prevents the loss of PEG caused by hydrolysis.

Materials: 1,2-Dioctadecyl-sn-glycerol: CAS: 82188-61-2, from BACHEM; Methoxy-PEG-NH2: Cat #12 2000-2, from RAPP Polymere; P-Nitrophenyl Chloroformate: CAS: 7693-46-1 from Aldrich

Synthetic Procedure: PEG-DSG-E is synthesized according to the route shown in FIG. 2C. Detailed procedures are described as follows.

Step 1: Activation of 1,2-dioctadecyl-sn-glycerol. Add p-nitrophenyl chloroformate (582 mg, 2.88 mmol, 1.05 equiv.) to a solution of 1,2-dioctadecyl-sn-glycerol (1.642 g, 2.75 mmol) and triethylamine (402.5 μl, 2.89 mmol) in 35 ml dichloromethane. Stir the reaction mixture at room temperature overnight. Analyze the crude mixture (RH1:79) by TLC (Hexane/Ethyl acetate, 3/1). TLC indicates that most of starting material 1,2-dioctadecyl-sn-glycerol is converted to the activated ester RH1:79.

Step 2: Conjugation of PEG. Pour a solution of methoxy-PEG-NH2 (5 g, 2.5 mmol) in 10 ml dichloromethane into the reaction mixture of RH1:79 at room temperature. Purge the mixture with Ar and stir the mixture at room temperature overnight. Concentrate the reaction mixture to about 10 ml. Precipitate the crude product by adding 80 ml anhydrous diethyl ether with vigorous stirring. Place the mixture at −20 Celsius for 1 hour, then filter and collect the filter cake. Dissolve the filter cake in 10 ml dichloromethane and precipitate the product again from 80 ml anhydrous diethyl ether at −20 Celsius. Dissolve the filter cake in 10 ml dichloromethane and load the solution on 80 g silica gel column. Purify the crude product by flash chromatography. Mobile phase A: chloroform, B: methanol. Elution segments: step 1: 0% B˜10% B 6 CV; step 2: 10%˜15% B 2 CV. Collect all peaks detected by UV and ELSD. Fractions #18˜22 are pooled as RH1:81A; #24˜40 as RH1:81B. Yield, RH1:81A, 2.5 g. RH1:81B, 1.8 g.

TLC analysis of the reaction mixture was performed with developing solvents: chloroform/methanol, 9/1, v/v. ¹H NMR spectrum indicates that RH1:81A is the desired conjugate.

### Example 1C Preparation of Liposomes

Liposomes are prepared by ethanol injection—extrusion method. For sphingomyelin (SM) liposomes, lipids are comprised of sphingomyelin, cholesterol at the molar ratio 3:2, and PEG-DSG in the amount of 6-8 mol % of sphingomyelin. Briefly, for a 30 ml liposome preparation, lipids are dissolved in 3 ml ethanol in a 50-ml round bottom flask at 70 Celsius. DEA-SOS (27 ml, 0.65-1.1N) is warmed at 70 Celsius water bath to above 65 Celsius and mixed with the lipid solution under vigorous stirring to give a suspension having 50-100 mM phospholipid. The obtained milky mixture is then repeatedly extruded, e.g., using thermobarrel Lipex extruder (Northern Lipids, Canada) through 0.2 μm and 0.1 μm polycarbonate membranes at 65-70° C. Phospholipid concentration is measured by phosphate assay. Particle diameter is analyzed by dynamic light scattering. Liposomes prepared by this method have sizes about 95˜115 nm.

### Example 1D Loading Method for Water Soluble Drugs

Step 1: Load DEA-SOS liposome (Less than 5% of the column volume) on the Sepharose CL-4B column equilibrated with deionized water. Wait for the complete absorption of liposome, then elute the column with deionized water and monitor the conductivity of the flow-through.

Step 2: Collect liposome fractions according to the turbidity of the flow-through. Majority of the liposome come out with a conductivity of 0. Discard the tailing fractions with conductivity higher than 40 μS/cm.

Step 3: Measure the liposome volume and balance the osmolarity immediately by adding 50 wt % dextrose into liposome to obtain a final concentration of 7.5-17 wt % dextrose, depending on DEA-SOS concentration inside the liposome. Buffers are chosen from their buffering pH range and capacity. Drug loading pH should not exceed 6.

Step 4: Adjust the pH of of the liposome by using concentrated buffers, HCl, and NaOH. Final buffer strength ranges from 5 mM to 30 mM.

Step 5: Analyze the lipid concentration by phosphate assay and calculate the amount of lipids needed for given input drug/lipid ratio.

Step 6: Prepare the drug solution in 7.5-17 wt % dextrose with the same buffer as used for the liposome solution. To enable comparisons between different prodrugs, the amount of drug added was based on docetaxel weight equivalents using a conversion factor to correct from the amount of prodrug salt form weighed out (Table 3).

Step 7: Mix drug and liposome solution to achieve the desired drug/phospholipid ratio (e.g., 150, 200, 300, 450, or 600 g docetaxel equivalents per mole phospholipid), then incubate at 70 Celsius (or desired temperature) for 15˜30 min with constant shaking.

Step 8: Chill the loading mixture on an ice-water bath for 15 min.

Step 9: Load part of the liposomes on a PD-10 column equilibrate with MES buffer saline (MBS) pH 5.5, or citrate buffer saline pH 5.5, or HBS pH 6.5 and eluted with the same buffer, and collect the liposomes. Keep both the purified and unpurified liposomes for next step analysis.

Step 10: Measure phospholipid concentration by phosphate assay for both before and after column samples.

Step 11: Analyze the drug concentration by HPLC for both before and after column samples.

Step 12: Encapsulation efficiency is calculated as: [drug/phospholipid (after column)]/[drug/phospholipid (before column)]*100 and described as grams of drug/mol phospholipid.

The amount of drug loaded in the liposome is expressed as docetaxel equivalents per mol phospholipid in the liposomes. To calculate number of docetaxel equivalents in a given amount of amino docetaxel hydrochloride salt, multiply the conversion factor from Table 3 below with the weighed out amount the salt. For example, 1 g of compound 1 is equivalent to 1 g×0.786=0.786 g of docetaxel. Similarly 2 g of compound 3 is equivalent to 2 g×0.819=1.638 g of Docetaxel.

TABLE 3

| | MW | MW | |
|---|---|---|---|
| | Free Base | HCl Salt | Conversion |
| Compd. | (grams/mol) | (grams/mol) | Factor |
| Docetaxel | 807.9 | | |
| 1 | 991.4 | 1027.6 | 0.786 |
| 2 | 977.2 | 1013.6 | 0.797 |
| 3 | 949.1 | 985.6 | 0.819 |
| 4 | 921.1 | 957.5 | 0.844 |
| 5 | 935.1 | 971.5 | 0.832 |
| 6 | 935.1 | 970.5 | 0.832 |

### Example 1E Method for Loading Drugs Poorly Soluble in Water (Less Than 1 mg/ml) by Using Short Chain Polyethylene Glycol

In this method, hexa(ethylene glycol) (PEG6) is used as an example for solubilizing and loading of drugs having very low water solubility (less than 1 mg/ml). It is believed that other short chain PEGs with molecular weight 200˜500 Daltons might also work for the same purpose. Solubilize drugs in 80% PEG6 in deionized water (by volume) at 40 mg/ml. Pre-warm liposomes to 70 Celsius and stir the solution constantly before drug loading. Then add the drug PEG6 solution to the liposome in small portions 8 times over 8 mins with 1 min interval. Incubate the loading mixture at 70 Celsius for another 22 min and cool on ice bath for 15 min. Separate free drug from liposome on a PD-10 column by using pH 5.5 MES buffer saline or pH 5.5 citrate buffer saline as the elution solution. Determine drug/lipid ratios before and after column by phosphate assay and HPLC analysis. Calculate encapsulation efficiency as:

[drug/phospholipid (after column)]/[drug/phospholipid (before column)]*100

### Example 1F Method for Loading Drugs by Using PEG400

In this example, PEG400 is used to replace more expensive PEG6 as the solubilizing agent for taxane prodrugs. This method is exemplified by the protocol for preparing compound 4 liposomes.

Part 1: Preparation of drug solution

1. Weigh 395 mg compound 4 in a 250 ml glass bottle.

2. Add 10 ml 80% PEG400, pH 2.8 solution followed by the addition of 134 μl of 3 M HCl solution.

3. Warm the above mixture at 50° C. water bath for about 10 min with intermittent shaking. A clear solution of compound 4 is obtained.

4. Dilute the solution 10× by adding 90 ml 5 mM MES 5% dextrose pH 3.8 solution. Final solution pH is 4.5.

5. Warm up the diluted solution to 65° C., and filter the solution through 1 μm PES membrane.

Part 2: Preparation of the SOS-liposome

1. Liposome formulation: SM/Chol/PEG-DSG=3/2/0.24 mol/mol/mol, 1.1 M DEA-SOS, Size: 99.7 nm.

2. Remove the external SOS by gel chromatography on Sepharose CL-4B with residual conductivity no more than 40 μS/cm.

3. Add 45wt % dextrose to the liposome to obtain a final 12% dextrose.

4. Add 1 M pH 5.4 citrate solution to the liposome to a final 20 mM

5. Determine the phosphate concentration by phosphate assay

Part 3: Loading of compound 4

1. Mix compound 4 solution with liposome prepared in part 2 at drug/lipid ratio of 600 g/mol at room temperature.

2. Pump the mixture through the heat exchanger made by Teflon thin-wall tubing at 70° C. to make sure the mixture is warmed up above 65° C. within 2 min.

3. Incubate the loading mixture at 70° C. for 30 min with stirring.

4. Cool the loaded liposome rapidly by pumping them through the heat exchanger submerged in ice water.

Part 4: Purification and concentration

1. Remove the free compound 4 by tangential flow filtration (TFF) and exchange the buffer to citrate buffer saline pH 5.5

2. Concentrate the liposome by TFF, then filter liposome through 1 μm, 0.45 μm, and 0.2 μm PES membranes sequentially.

### Example 1G Preparation of Antibody-Lipid Conjugates

Antibody-PEG-lipid conjugates are used to form antibody-linked liposomes. They can be prepared starting with the scFv protein expressed in a convenient system (e.g. mammalian cell) and purified, e.g., by the protein A affinity chromatography, or any other suitable method. In order to effect conjugation, scFv protein is designed with a C-terminal sequence containing a cysteine residue. Preparation of scFv-PEG-lipid conjugates, such as scFv-PEG-DSPE, is described in the literature (Nellis et al. Biotechnology Progress, 2005, vol. 21, p. 205-220; Nellis et al. Biotechnology Progress, 2005, vol. 21, p. 221-232; U.S. Pat. No. 6,210,707). For example, the following protocol can be used:

Step 1: Dialyze the scFv protein stock solution against pH 6.0 CES buffer (10 mM sodium citrate, 1 mM EDTA, 144 mM sodium chloride) at 4° C. for 2 h.

Step 2: Reduce the antibody in the pH 6.0 CES buffer in the presence of 20 mM 2-mercaptoethanamine at 37° C. for 1 h.

Step 3: Purify the reduced antibody on a Sephadex G-25 column.

Step 4: Incubate reduced antibody with 4 mole excess of maleimide-PEG-DSPE in pH 6.0 CES buffer at room temperature for 2 h. Quench the reaction by adding cysteine to a final concentration of 0.5 mM.

Step 5: Concentrate the conjugation mixture on an Amicon stir cell concentrator.

Step 6: Separate the conjugate from free antibody on an Ultrogel AcA44 column.

Step 7: Analyze the conjugate by SDS-PAGE.

### Example 1H Preparation of Targeted Liposomes

Antibody-targeted liposomes can be prepared by incubating antibody-PEG-lipid conjugates (Example 1G) with liposomes in an aqueous buffer at 37° C. for 12 h or at 60° C. for 30 min depending on the thermal stability of the antibody. The lipid portion of a micellar conjugate spontaneously inserts itself into the liposome bilayer. See, e.g., U.S. Pat. No. 6,210,707, filed May 12, 1998 and incorporated herein by reference. The ligand inserted liposomes are purified, e.g., by size exclusion chromatography on a Sepharose CL-4B column and analyzed by phosphate assay for lipid concentration and SDS-PAGE for antibody quantification.

### Example 1I Preparation of Tritium Labeled Liposomes

Drug loaded liposomes with a non-exchangeable tritium labelled lipid, ³H-cholesterlyl hexadecyl ether (³H-CHDE) in the lipid bilayer (tritium-labeled liposomes) allow simultaneous monitoring the pharmacokinetics of both drug and lipids. Tritium-labeled liposomes of different formulations with various trapping reagents are prepared by extrusion method. The general protocol for preparing tritium-labeled “empty” liposomes (i.e., the liposomes that do not contain the drug) can be, for example, as follows.

1. Clean a 12-ml glass vial with chloroform/methanol (2/1, v/v), then acetone, and dry the vial by heat gun.

2. Transfer 1 ml commercially available ³H-CHDE solution in toluene to the glass vial. Dry the solution with a stream of argon at room temperature for 30 min, leave the vial under the oil pump vacuum overnight.

3. Weigh the lipids according to the formulation, and add them into the vial containing ³H-CHDE.

4. Add 1 mL 200-proof ethanol into the lipids vial and heat up to 70° C. to dissolve the lipids till a clear solution is obtained.

5. Add the warm lipid solution to 10 mL pre-warmed trapping reagent solution. Keep stirring the mixture at 70° C. for 15 min to produce multi-lamellar vesicles (MLV).

6. Repeatedly pass the MLVs through polycarbonate track-etched membrane filters with appropriate pore size (for example, 0.2 μm, 0.1 μm, and 0.08 μm) at 70° C. until the desired liposome size (e.g., about 110 nm) is achieved. Keep the extruded liposomes at 4° C.

Docetaxel prodrugs are loaded into tritium-labeled liposomes according to methods described in Examples 1D-F depending on drug's properties. Targeting antibody are inserted into drug loaded liposomes by the method described in Example 1H.

### Example 2: Drug Biodistribution of EphA2 Targeted Docetaxel-Generating Nanoliposome Compositions and Docetaxel in a Triple Negative Breast Cancer Xenograft Model in Mice

This example describes the drug biodistribution of an EphA2-targeted docetaxel-generating immunoliposome and docetaxel were studied in the xenograft models of human triple negative breast cancer MDA-MB-231.

MDA-MB-231 cells were obtained from Asterand Bioscience and propagated in RPMI medium supplemented with 5% fetal calf serum, 10 mM HEPES, 5 μg/ml Insulin, 1 μg/ml Hydrocortisone, 50 U/ml penicillin G, and 50 μg/mL of streptomycin sulfate at 37° C., 5% CO2.

The EphA2-ILs-DTX immunoliposome was 40scFv-ILs-DTXp3, with a lipid portion composed of egg sphingomyelin, cholesterol (3:2 molar ratio) and 5 mol % PEG-DSG, containing entrapped 1.1N TEA-SOS, prepared and loaded with Compound 3 at the drug/lipid ratio of 315 docetaxel equivalents/mole phospholipid as described in Example 1, and the scFv of SEQ ID NO:40 attached to the outside of the liposome.

SCID beige homozygous female mice (5-6 week old) were obtained from Charles River. The mice were inoculated orthotopically into the mammary gland-right thoracic fat pad with 0.08 mL of the suspension containing 0.5×10⁶ cells suspended in PBS supplemented with 30% growth factor reduced Matrigel® Matrix (Corning, cat. #354230). When tumors achieved the size between 200 mm³ and 350 mm³ the animals were assigned to the treatment groups according to the following method. The animals were ranked according to the tumor size, and divided into 6 categories of decreasing tumor size. Treatment groups of 4 animals/group were formed by randomly selecting one animal from each size category, so that in each treatment group all tumor sizes were equally represented. Treatment arms and sacrifice time points are summarized in table 4. Each group consists of 3 animals. “Mpk” stand for mg of the drug per kg animal body weight.

TABLE 4
Experimental Design

| Groups | Treatment | Dose | Route | Frequency | Time points (n = 5/TP) | Tissue |
|---|---|---|---|---|---|---|
| 1 | 40scFv-ILs-DTXp3 | 50 mpk | IV | 1x | 2 hr, 6 hr, 24 hr, Day 3, | Tumor, Liver, |
| | | | | | Day 7, Day 10 | Spleen |
| 3 | Docetaxel | 10 mpk | IV | 1x | 2 hr, 6 hr, 24 hr, Day 3, | |
| | (free drug) | | | | Day 7, Day 10 | |

The animals received one tail vein injection of 40scFv-ILs-DTXp3 at 50 mpk or free docetaxel at 10 mpk. At each time point, animals were sacrificed and tumor, liver and spleen were collected and flash frozen.

All tissue samples were thawed, weighed, and then mechanically homogenized using a Bullet Blender (Next Advance, Inc., Averill Park, N.Y.) in a solution of 40% acetonitrile containing 0.25% trifluoroacetic acid. The acid was included in the solvent for pH stabilization of Compound 3. The target tissue concentration was 200 mg of tissue per milliliter of total homogenate (i.e. tissue plus solvent); however, in the case of low tissue weights (<100 mg), the final tissue concentration was 100 mg/mL.

The homogenized tissue samples were analyzed utilizing two bioanalytical assays to individually measure Compound 3 and DTX. Both methods employ a high pressure liquid chromatographic assay with tandem mass spectrometric detection (LC-MS/MS). The analytes, Compound 3 and DTX, were detected in both assays by multiple reaction monitoring (MRM) in positive ion mode using electrospray ionization. The homogenized tissue samples were quantitated for Compound 3 and DTX using extracted calibration standards and quality control (QC) samples prepared by spiking Compound 3 or DTX into an acidified plasma matrix, which is a 3:2 mixture of female CD-1 lithium heparinized mouse plasma (Bioreclamation, LLC, Westbury, N.Y.) and 200 mM sodium phosphate buffer (pH 2.5). The calibration ranges for Compound 3 and DTX are 15.0-5,000 ng/mL and 3.00-1,000 ng/mL, respectively. Acidified plasma QCs were prepared at 45.0, 375 and 3,750 ng/mL for Compound 3 and 9.00, 75.0 and 750 ng/mL for DTX. Calibration curves were generated using the analyte/internal standard (IS) peak area response ratios versus nominal concentrations (ng/mL) and weighted linear regressions with a weighting factor of 1/concentration².

Sample extraction for Compound 3 utilizes a protein precipitation by mixing a 50 μL aliquot of the tissue homogenate with 200 μL of acidified acetonitrile that contains the IS, paclitaxel. The resulting solution is vortexed thoroughly and then centrifuged for 10 minutes at 14,000 rpm and 4° C. A 50 μL aliquot of the supernatant is removed and mixed with 200 μL of water containing 12% acetonitrile and 0.1% formic acid in an autosampler vial. A 10 μL aliquot of the resulting solution is injected. LC-MS/MS analysis of the extracted Compound 3 samples is performed using a Finnigan Surveyor MS Pump Plus system with a CTC Analytics PAL autosampler and TSQ Quantum Ultra mass spectrometer (Thermo Scientific, Waltham, Mass.). Chromatographic separation is achieved on an Atlantis dC18, 2.1×150 mm column (Part Number: 186001301, Waters Corporation, Milford, Mass.) using a gradient of water/acetonitrile containing formic acid with a 30° C. column temperature, a flow rate of 400 μL/min, and a run time of 11.0 minutes.

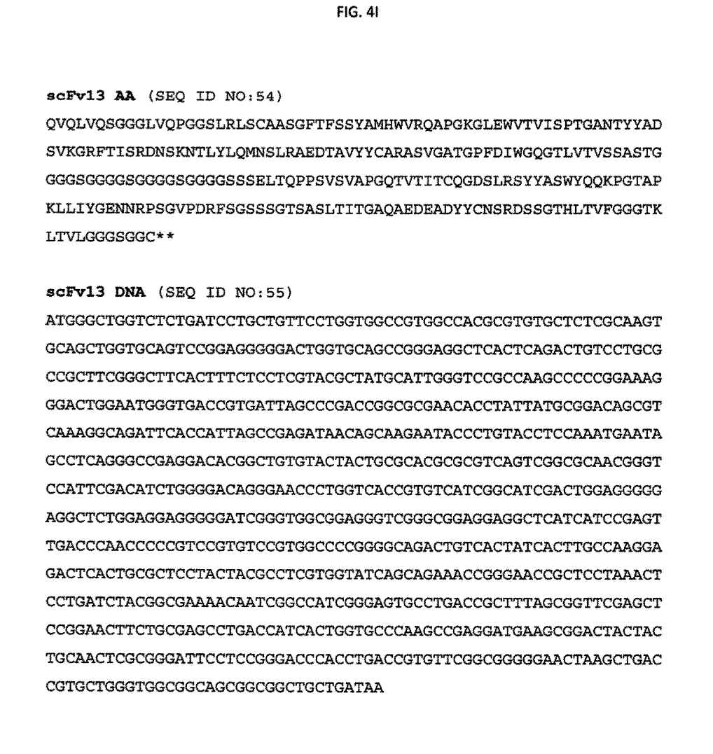

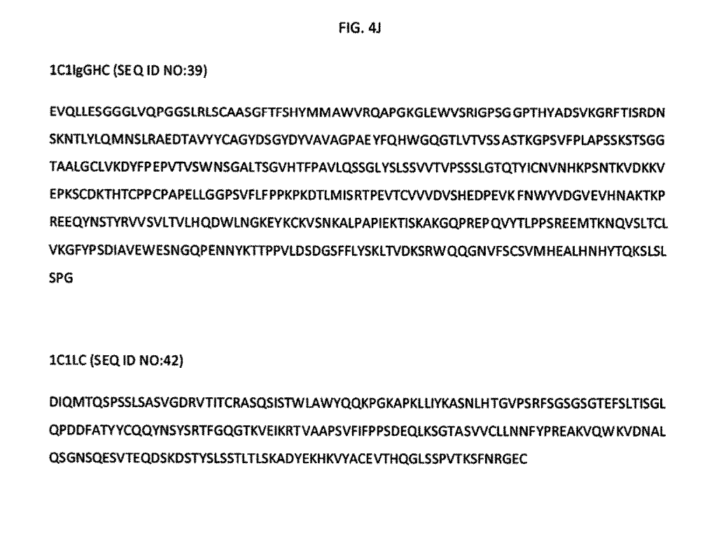

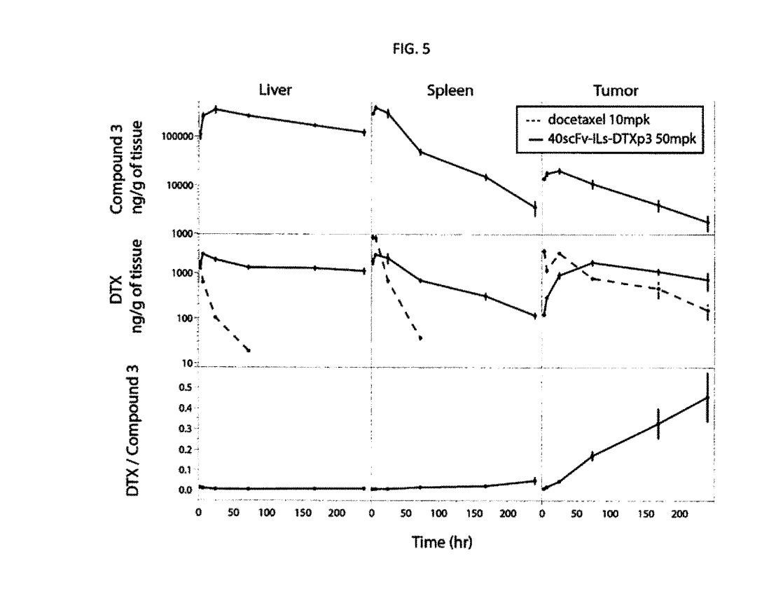

The analysis for docetaxel (DTX) utilizes a liquid-liquid extraction (LLE) procedure where a 50 μL aliquot of homogenized tissue, 50 μL of paclitaxel IS in acidified methanol, 900 μL of 1% trifluoroacetic acid, and 4.0 mL of n-butyl chloride:acetonitrile (4:1) are added together in a glass screw-top tube. The tubes were capped with Teflon-lined caps, vortexed, rotated for 15 minutes, and then centrifuged at 3,000 rpm and 4° C. for 15 minutes to separate the liquid phases. After freezing the aqueous phase in a dry ice/acetone bath, the n-butyl chloride:acetonitrile layer is poured off into a new glass screw-top tube and evaporated at 35° C. under nitrogen. The residue is reconstituted in 75 μL of 30% acetonitrile with 2.5 M formic acid and centrifuged at 3,200 rpm and 4° C. for 20 minutes to separate insoluble materials. The resulting supernatant was transferred to an autosampler vial and a 20 μL aliquot injected. LC-MS/MS analysis of the extracted DTX samples is performed using a Agilent 1200 series HPLC system (Agilent Technologies, Santa Clara, Calif.) and an AB Sciex API 3000 mass spectrometer (AB Sciex, Framingham, Mass.). Chromatographic separation is achieved on an Atlantis dC18, 2.1×150 mm column (Part Number: 186001301, Waters Corporation, Milford, Mass.) using a gradient of water/acetonitrile containing formic acid with a 30° C. column temperature, a flow rate of 400 μL/min, and a run time of 13.0 minutes. FIG. 5 is a graph showing DTX (docetaxel) and Compound 3 (docetaxel prodrug) levels in tumor, spleen and liver measured in MDA-MB-231. 40scFv-ILs-DTXp3 shows prolonged exposure in the tumor as seen in Compound 3. When compared to free docetaxel, 40scFv-ILs-DTXp3 led to an accumulation of docetaxel starting at later time point but was sustained up to day 10 (240 hrs). The change in docetaxel/Compound 3 ratio suggests sustained conversion of the Compound 3 to docetaxel at the tumor site but to a lesser extent in liver and the spleen.

### Example 3: EphA2-Targeted Liposomes Specifically Bind and Penetrate EphA2-Expressing Cells Cultured as Spheroids

In this study, we established an in vitro three-dimensional (3D) model of liposome penetration into tumors for the purpose of analyzing the effect of liposome binding to the target on penetration depth. We found that EphA2 targeting was highly specific and liposome-spheroid association was only seen in the EphA2 targeted liposome when incubated with EphA2+ cells. The test liposomes comprising fluorescent lipid labels DilC18(3)-DS and DilC18(5)-DS, were prepared as described in Example 4 below.

Cell Culture

BT474-M3 cells were a gift from Hermes Bioscience (San Francisco, Calif.) OVCAR-8 cells were obtained from the National Cancer Institute (Bethesda, Md.). NCI-H1993 were from the American Type Culture Collection (Manassas, Va.). Cell lines were cultured in RPMI; culture medium for cell passage was supplemented with 10% FBS plus penicillin/streptomycin, and passaging for routine culture was by trypsinization. Culture medium used for all imaging experiments was phenol red free RPMI. Cells were incubated for culture or IncuCyte ZOOM™ experiments in 5% CO2 at 37° C.

Test for Spheroid Formation

To determine whether a cell line would grow as a spheroid, 5000 cells were plated in Costar 7007 U-bottom, ultra-low adhesion 96-well plates (Corning, Corning N.Y.) in complete medium and allowed 4 days of culture to establish spheroids. This method of plating cells was used for all subsequent experiments with spheroids.

Test for Evidence of Receptor Targeting in Liposome Uptake

Complete culture medium was removed from plates after gently spinning down cells to bottom of wells, and replaced with serum-free, phenol-red-free medium (supplemented with penicillin/streptomycin) containing placebo non drug loaded fluorescently tagged liposome (DilC18(3)—Thermofisher Scientific, D-282) DilC18(3)-Ls were either targeted (46scFv-ILs) or non-targeted (NTLs) at a final concentration of 25 μM phospholipid. Negative control wells received serum-free medium alone. Plates were spun briefly to center the spheroids at the bottom of the wells before loading into the IncuCyte ZOOM™. Scanning commenced within 1 hour of addition of liposomes and was scheduled to continue every 30-45 minutes for a minimum of 18 hours.

Test for Effect of Receptor Density in Liposome Uptake

Cell lines used were all capable of forming spheroids: NCI-H1993 (lung), OVCAR-8 (ovarian), and BT474-M3 (breast). Cultures in U-bottom plates were allowed to establish for 4 days before imaging liposome uptake in serum-free, phenol-red free medium as done to test for receptor targeting. Fluorescently-conjugated liposomes with targeting ligand 46scFV were applied at a final concentration of 25 μM phospholipid.

Analysis of Images from IncuCyte ZOOM™

Images were exported from the IncuCyte ZOOM™ in TIFF format with paired fluorescent and phase contrast images for each well at each time point. An in-house MATIAB (Mathworks, Natick Mass.) script was used to analyze the images. In summary, spheroids were segmented based on the phase-contrast image using edge detection algorithms, perimeter of the spheroid was further refined using morphological operators such as closing and hole filling, intensity of the red fluorescent signal in the matched fluorescent image file was extracted, and mean intensity of the red signal from the periphery of the spheroid was calculated. The relationship between mean fluorescent intensity and distance to the spheroid edge is used to estimate liposome penetration. AUC derived from a plot the distance from the periphery vs. signal strength was calculated for each measured time point.

Assay for Levels of EphA2 in Spheroid Cultures

Upon the completion of imaging experiments, 96 well culture plates were removed from IncuCyte ZOOM™ and set on ice. Spheroids from multiple wells were pooled and rinsed in cold PBS. Cells were lysed in chilled BioPlex lysis buffer (Bio-Rad, Hercules Calif.) 30 min at 4° C., then stored at −80° C. ELISA assays for human EphA2 were run according to manufacturer's instructions (R&D Systems, Minneapolis Minn.) and values expressed relative to total protein measured by BCA assay with a BSA standard (Pierce/ThermoFisher, Waltham Mass.).

Targeted Liposome Uptake by Spheroids Requires EphA2 Expression

Observations of liposome uptake was performed using the IncuCyte ZOOM™ data viewing tool. Fluorescent signal was first seen at the periphery of the spheroid and would extend toward the center with time. Liposome cell binding was only seen in EphA2+ cells and using EphA2-targeted liposomes. Non-targeted liposomes did not show fluorescence either on the surface or interior of the spheroids regardless of EphA2 levels. Cell lines with very low EphA2 expression (e.g. BT474-M3) did not take up spheroids over the ˜24 h time period allowed in these experiments.

Images acquired with the IncuCyte ZOOM™ were obtained for two cell lines (NCI-H1993, and BT474-M3) at the completion of a representative experiment in which the cells were allowed to form spheroids before incubation with targeted, Dil-5-labeled liposomes (23 h time point). Raw data files containing either the red fluorescent image or the phase contrast image were superimposed to show the extent of liposome uptake at the completion of a representative experiment. These images were created using the same scale for the fluorescent channel. Measured levels of EphA2 were 24 pg/μg total protein in NCI-H1993 spheroids and 2 pg/μg in BT474-M3 spheroids.

Uptake of liposomes with varying concentrations of targeting molecules relative to the phospholipid component were most clearly visualized in cell lines with the highest liposome uptake. NCI-H1993 spheroids at 25 h (study end) incubation with targeted liposomes.

Kinetics of EphA2-Liposome Penetration in 3D Tumor Spheroids

Analysis of the mean fluorescence intensity (MFI) showed a gradual liposome uptake that persisted until the last analyzed time point without reaching saturation. The gradual increase reflects both liposome binding/internalization by cells but also liposome penetrating deeper layers in the spheroid.

### Example 4: Microdistribution of EphA2 Targeted Liposome, Non-Targeted Liposome and EphA2 Antibody in Normal Tissues and Tumor in Esophageal Cancer Xenograft Model and Metastatic Breast Cancer Model in Mice

Microdistribution of EphA2 targeted non drug loaded liposome (46scFv-Ls), non-targeted liposome (NT-Ls) and EphA2 antibody 1C1 (SEQ ID NO:39 clone 1C1, disclosed in published PCT patent application PCT/US06/34894, filed Aug. 4, 2008) was studied in tumor and normal tissues of the xenograft model of human esophageal carcinoma OE-19 and in tumor and lung of metastatic model of triple negative breast cancer MDA-MB-231 .

OE-19 cells were obtained from European Collection of Cell Cultures (Salisbury, UK) Asterand Bioscience. MDA-MB-231 were obtained from Asterand Bioscience. Cells were propagated in RPM11640 medium supplemented with 5% fetal calf serum, 10 mM HEPES, 5 μg/ml Insulin, 1 μg/ml Hydrocortisone, 50 U/ml penicillin G, and 50 μg/mL of streptomycin sulfate at 37° C., 5% CO2.

For OE-19 model, NCR nu/nu homozygous athymic male nude mice (5-6 week old) were obtained from Taconic. Five mice per group were inoculated ectopically in the flank with 0.1 mL of the suspension containing 10×10⁶ cells suspended in PBS. When tumors achieved the size between 150 mm³ and 350 mm³ the animals were injected with liposome mixture or EphA2 IgG antibody (1C1, included as SEQ ID NO:39).

Two models of metastatic triple negative breast cancer were used based on MDA-M-231 cell line. 1) Metastatic lesions generated through intravenous inoculation of cancer cells (MDA-MB-231 IV model) 2) metastatic lesions generated through orthotopic implantation of low number of cancer cells in the mammary fat pad which provides enough time for dissemination and lesion development in lung (MDA-MB-231 Orthotopic).

For MDA-MB-231 IV model, NCR nu/nu homozygous athymic female nude mice (5-6 week old) were obtained from Taconic. 0.5×10⁶ MDA-MB-231 cells suspended in PBS were injected intravenously through the tail vein in three mice. Animals were sacrificed at four weeks post cell injection, and lungs were extracted for analysis of metastatic lesions.

For MDA-MB-231 Orthotopic model, SCID Beige female mice (5-6 week old) were obtained from Taconic. Five mice were inoculated bilaterally orthotopically in the mammary fat pad. For each gland, 0.2 mL of the suspension containing 0.5×10⁶ cells suspended in PBS supplemented with 30% growth factor reduced Matrigel® Matrix (Corning, cat. #354230). To increase the probability of metastases, each animal was inoculated bilaterally and had two primary tumors. Animals were sacrificed at eight weeks post cell inoculation and tumor and lungs were extracted for analysis of primary tumor and metastatic lesions

In order to test the effect of targeting on liposome microdistribution in vivo, 24 hours before sacrifice, animals were injected with a mixture of EphA2 targeted non drug loaded placebo liposomes and a non-targeted non drug loaded placebo liposomes at dose of 1 micromole/liposome type/animal labeled with two different lipophilic fluorescent dyes DilC18(5)-DS and DilC18(3)-DS, respectively (EphA2-ILs/NT-Ls group) To control for differences in fluorescence between DilC18(5) and DilC15(3), one animal from each model was injected with the a mixture of non-targeted liposomes which should distribute equally in tissue (NT-Ls/NT-Ls group).

A second group of animals was injected with the anti-EphA2 antibody clone 1C1 (SEQ ID NO:39) at 5 mg/kg bw.

Non drug loaded liposomes are prepared by ethanol injection—extrusion method. For sphingomyelin (SM) liposomes, lipids are comprised of sphingomyelin, cholesterol and PEG-DSG (3:2:0.24 molar parts), with either DilC18(3)-DS (Dil3-Ls), or DilC18(5)-DS (Dil5-Ls) fluorescent lipid labels added at a ratio of 0.3 mol % of the total phospholipid. Briefly, for a 30 ml liposome preparation, lipids are dissolved in 3 ml ethanol in a 50-ml round bottom flask at 70 Celsius. HEPES-buffered saline (5 mM HEPES, 144 mM NaCl, pH 6.5) is warmed at 70 Celsius water bath to above 65 Celsius and mixed with the lipid solution under vigorous stirring to give a suspension having 50-100 mM phopsholipid. The obtained milky mixture is then repeatedly extruded, e.g., using thermobarrel Lipex extruder (Northern Lipids, Canada) through 0.2 μm and 0.1 μm polycarbonate membranes at 65-70° C. Phospholipid concentration is measured by phosphate assay. Particle diameter is analyzed by dynamic light scattering. Liposomes prepared by this method have sizes about 95˜115 nm. Anti-EphA2 scFv proteins were expressed in mammalian cell culture, purified by protein A affinity chromatography, and conjugated through C-terminal cysteine residue to maleimide-terminated lipopolymer, mal-PEG-DSPE, in aqueous solution at 1:4 protein/mal-PEG-DSPE molar ratio. The resulting micellar scFv-PEG-DSPE conjugates were purified by gel chromatography on Ultrogel AcA34 (Sigma, USA). Targeted Dil3-Ls or Dil5-Ls were prepared by incubation with micellar anti-EphA2 scFv-PEG-DSPE conjugate at 60° C. for 30 min depending on the thermal stability of the antibody at the scFv/liposome ratio of 10-12 g/mol phospholipid for 40scFv-ILs, and 5 g/mol phospholipid for 46scFv-ILs. The ligand inserted liposomes are purified on Sepharose CL-4B column and analyzed by phosphate assay for lipid concentration and SDS-PAGE for antibody quantification.

For OE-19 model, 46scFv-ILs were used. For MDA-MB-231 models, 40scFv-ILs liposomes were used (All tissues were collected 24 h after injection. Mice were sacrificed one at a time by asphyxiation with CO2, and tissues perfused with 10-15 ml PBS immediately before collection. Excised tissues were frozen in OCT medium with three cryomolds per animal: 1) heart, lung, liver, spleen; 2) tumor, colon, kidney, brain; 3) skin. All frozen tissues and cryostat sections were stored at −80° C. For MDA-MB-231 orthotopic model, lung and tumor were excised and frozen in one OCT block. For MDA-MB-231 IV model, only lungs were excised and frozen in one OCT block per animal. For the liposomes injected group, frozen sections were allowed to set at room temperature 5-10 minutes before fixation with 4% paraformaldehyde, 10 min. Samples were rinsed with TBS, and nuclei counterstained with Hoescht 33342 (Invitrogen/ThermoFisher, Grand Island N.Y.) diluted in DaVinci Green Diluent (BioCare Medical, Concord Calif.). Samples were rinsed with TBS, mounted in Prolong Gold (Invitrogen), and allowed to set overnight before imaging.

For 1C1 group (SEQ ID NO:39), additional steps were performed to stain the antibody. In summary, cryostat sections were allowed to sit at room temperature 5-10 minutes before fixation with cold NBF, 10 min. Samples were rinsed with TBS-T, then nonspecific binding blocked with Background Sniper (BioCare), 10 min. Following another rinse with TBS-T, human IgG was detected with 5 μg/ml goat-anti-human IgG (gamma-chain specific; Vector Laboratories, Burlingame Calif.) in DaVinci Green diluent, with overnight incubation in a humidity chamber at 4° C. Serial sections of control slides were incubated in parallel with an equal concentration of goat anti-rabbit IgG. After washing with TBS-T, bound anti-human or anti-rabbit antibodies were detected with 20 μg/ml Alexa647-conjugated donkey anti-goat IgG H+L (Invitrogen), 30 min, room temperature. These samples were counterstained with Hoescht 33342 and mounted in Prolong Gold as described for visualization of liposomes.

Images were collected with an AperioFL Scanscope (Leica Biosystems, Buffalo Grove Ill.) at 20× magnification. Each channel was collected independently with filters for DAPI (Hoescht-stained nuclei), SPOR-B (Dil3 liposomes), and CY5 (Dil5 liposomes, Alexa647-conjugated antibodies). For visualization of human IgG 1C1, control slides stained with anti-rabbit IgG were imaged with the same settings used for the test samples.

Quantitation of liposome deposition and comparison of the extent of overlap of targeted and non-targeted liposomes was done using an in-house algorithm and user interface developed using Matlab (Mathworks, Natick, Mass.). In summary, tissue area within tumor or normal organs determined by segmenting nuclear staining was subdivided in tiles of 200×200 μm and mean intensity of EphA2-targeted liposome and non-targeted liposome extracted from the corresponding fluorescent image. Ratiometric imaging was performed by calculating the ratio of two channels at the single pixel level. Images were opened at the resoltuon of 10 micron per pixel, ratios were computed than a median filter with a size ranging from 3 to 10 was applied to filter out noise. Given that clone 1C1 (SEQ ID NO:39) were injected into separate animals, the comparison between liposomes and IgGs was done using visual inspection.

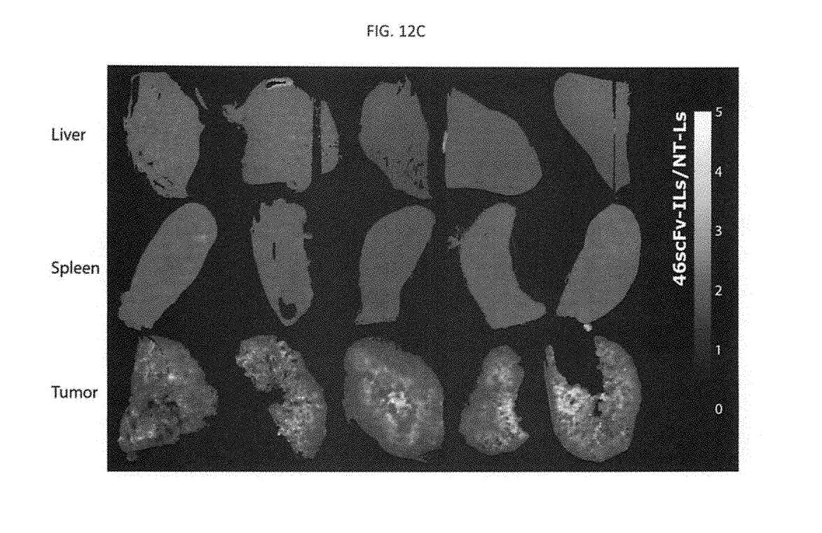

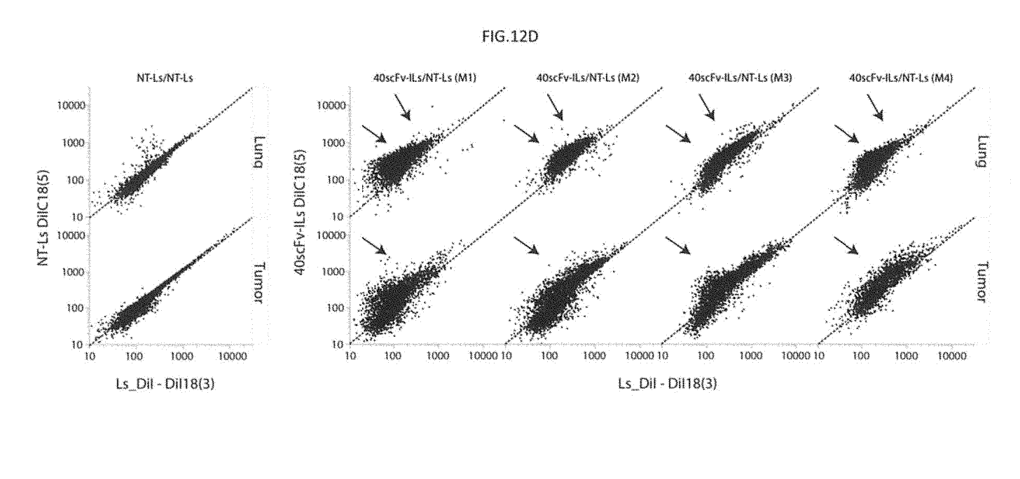

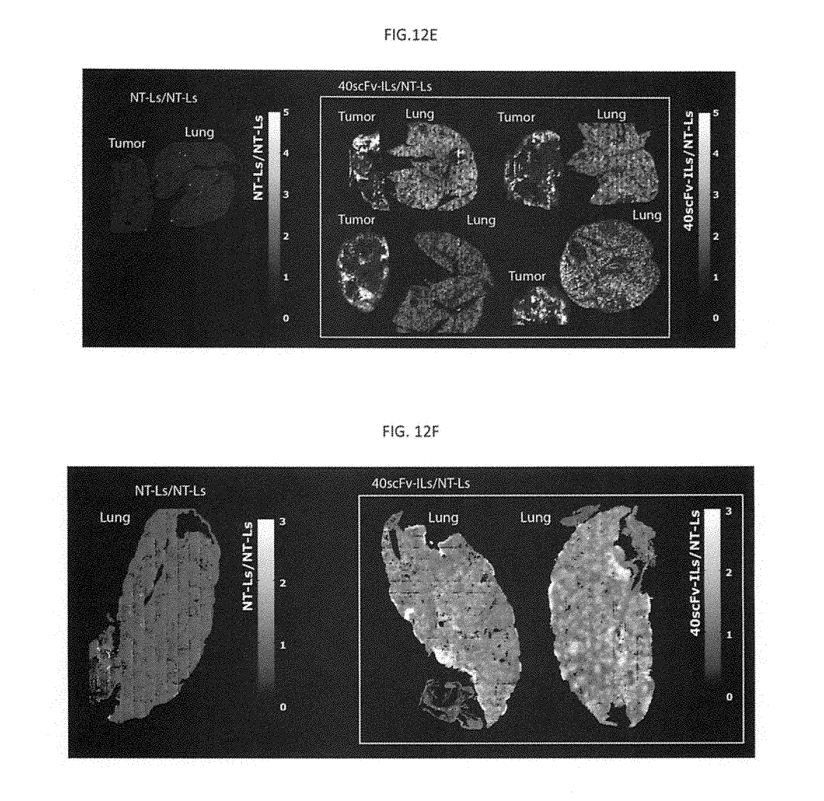

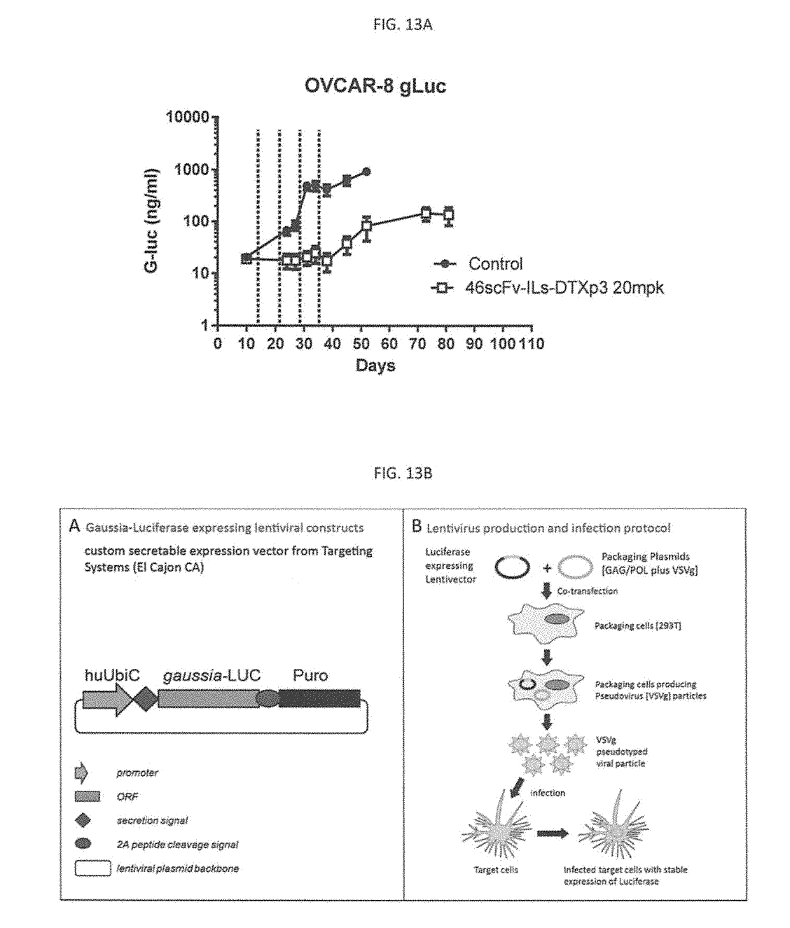

In OE-19 model, analysis of overall liposome deposition (NT-Ls and 46scFv-ILs) within organs showed preferential biodistribution is specific organs including liver, spleen and tumor and significantly lower levels in all the other collected organs (FIG. 12A). Liposomes were deposited in specific tissue sub-compartments within the liver and the spleen and at lower levels in the dermal and subcutaneous area of the skin. In normal organs, EphA2 targeting did not significantly affect the microdistribution of the liposome and 46scFv-ILs and NT-Ls colocalized in the same areas in most analyzed organs. To analyze target mediated shift in microdistribution of the liposome, we performed ratiometric analysis of the images. This analysis is only meaningful in tissue in which the liposome deposition is high enough minimizing erroneous ratios. Thus, we performed ratiometric analysis for liver, spleen and tumor. Unlike normal tissues, in tumors EphA2 targeting did impact liposome microdistribution. Analysis of the 46scFv-ILs localization compared to that of the non-targeted liposome shows a significant shift in microdistribution, with some areas having 2 to 3 fold more targeted than non-targeted liposome. Quantification of the spatial distribution and colocalization of liposome mean fluorescent intensity shows a significant shift across the diagonal line toward the 46scFv-ILs in all animals. The shift was seen mostly in lower deposition areas (FIG. 12B). Analysis of ratiometric images in which the ratio of 46svFv-ILs over NT-Ls is displayed and shows higher ratios in the tumor specifically in the core.

FIG. 12B is a graph showing liposomal ratiometric analysis of EphA2 targeting. FIG. 12C is an image showing liposomal ratiometric analysis of EphA2 targeting.

At the organ level, 1C1 antibody (SEQ ID NO:39) did biodistribute to most analyzed organs in a very diffuse way which differs from the pattern of deposition of the NT-Is or EphA2-KLs. 1C1 (SEQ ID NO:39) showed high staining in the epidermis, while 46svFv-ILs were mainly located in the dermis.

Both metastatic models led to the appearance of multiple lesions of different sizes in the lung. MDA-MB-231 IV has extensive metastatic burden in which most of the lung is replaced by infiltrative diffuse tumors. In MDA-MB-231 Orthotopic, metastatic burden was less than MDA-MB-231 IV and was discrete through appearance of histologically identifiable micro or macro lesions.

In both models, analysis of the animals of the NT-Ls/NT-Ls group showed deposition of both liposomes in the same areas of the tumor or of the metastatic lesions. Quantification of the spatial distribution & colocalization of liposome mean fluorescent intensity shows a significant shift across the diagonal line toward the 40scFv-ILs in all animals (FIG. 12D). Ratiometric analysis showed a ratio close to one. In primary tumors EphA2 targeting did impact liposome microdistribution. Analysis of the 40scFv-ILs localization compared to that of the non-targeted liposome shows a significant shift in microdistribution, with some areas having 2 to 5 fold more targeted than non-targeted liposome (FIG. 12E). A similar pattern was seen in lung metastatic lesions in which the 40svFv-ILs deposited diffusely in the lung in metastatic lesions or peri lesion areas. This pattern of liposome deposition in metastatic lung lesions was seen in both models MDA-MB231 IV (FIG. 12F) and MDA-MB-231 Orthotopic (FIG. 12E).

### Example 5: In Vivo Antitumor Efficacy of 40scFv-ILs-DTXp3 in Triple Negative Breast Cancer Xenografts in Mice

Antitumor efficacy of 40scFv-ILs-DTXp3 was studied in the xenograft models of human triple negative breast cancer (TNBC) cell lines SUM-159, SUM-149 and MDA-MB-436. MDA-MB-436 were obtained from American Type Culture Collection (Rockville, Md.) and propagated in L15 medium supplemented with 10% fetal calf serum, 50 U/ml penicillin G, and 50 μg/mL of streptomycin sulfate at 37° C., 5% CO2. SUM-159 and SUM-149 cell lines were obtained from Asterand Bioscience and propagated in Ham's F-12 medium supplemented with 5% fetal calf serum, 10 mM HEPES, 5 μg/ml Insulin, 1 μg/ml Hydrocortisone, 50 U/ml penicillin G, and 50 μg/mL of streptomycin sulfate at 37° C., 5% CO₂.

NCR nu/nu homozygous athymic male nude mice (4-5 week old, weight at least 16 g) were obtained from Taconic. The mice were inoculated orthotopically into the mammary gland-right thoracic fat pad with 0.1 mL of the suspension containing 10×106 cells suspended in PBS supplemented with 30% growth factor reduced Matrigel® Matrix (Corning, cat. #354230). When tumors achieved the size between 150 mm³ and 350 mm³ the animals were assigned to the treatment groups according to the following method. The animals were ranked according to the tumor size, and divided into 6 categories of decreasing tumor size. Treatment groups of 8 animals/group were formed by randomly selecting one animal from each size category, so that in each treatment group all tumor sizes were equally represented.

The following treatment arms have been formed, for SUM-159 and SUM-149 xenograft models: 1) Control (HEPES-buffered saline pH 6.5); 2) Docetaxel at dose 10 mg/kg per injection; 3) 40scFv-ILs-DTXp3 at dose 50 mg/kg per injection.

For MDA-MB-436 xenograft model: 1) Control (HEPES-buffered saline pH 6.5); 2) Docetaxel at dose 10 mg/kg per injection; 3) 40scFv-ILs-DTXp3 at dose 12.5 mg/kg per injection; 4) 40scFv-ILs-DTXp3 at dose 25 mg/kg per injection, 5) 40scFv-ILs-DTXp3 at dose 50 mg/kg per injection.

The animals received four tail vein injections, at the intervals of 7 days.

The EphA2-ILs-DTX immunoliposome was 40scFv-ILs-DTXp3, with a lipid portion composed of egg sphingomyelin, cholesterol (3:2 molar ratio) and 8 mol % PEG-DSG, containing entrapped 1.1N TEA-SOS, prepared and loaded with Compound 3 at the drug/lipid ratio of 450 docetaxel equivalents/mole phospholipid as described in Example 1, and the scFv of SEQ ID NO:40 attached to the outside of the liposome.

Injection concentrated Docetaxel (Accord Healthcare Inc., 20 mg/ml alcohol solution) was dissolved prior administration in PBS to 1 mg/ml.

The animal weight and tumor size were monitored twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume=[(length)×(width)²]/2

To assess treatment-related toxicity, the animals were also weighted twice weekly. When the tumors in the group reached 10% of the mouse body weight, the animals in the group were euthanized. Average tumor volumes across the groups were plotted together and compared overtime.

As shown in in FIGS. 7A, 7B and 7C, 40scFv-ILs-DTXp3 has a significantly stronger antitumor efficacy comparing to the equal toxic dose of docetaxel in all three xenograft models. The treatment related toxicity was assessed by the dynamics of animals' body weight (FIGS. 7D, 7E and 7F). Neither group revealed any significant toxicity in to relatively slow growing models: SUM-149 and MDA-MB-436. The weight of the animals in all treated groups was comparable to the control group and was consistently increasing. While severe docetaxel and moderate 40scFv-ILs-DTXp3 toxicity was observed in more aggressive model, SUM-159.

FIG. 7A is a graph showing the antitumor efficacy 40scFv-ILs-DTXp3 in SUM-159 xenograft model. FIG. 7B is a graph showing the antitumor efficacy 40scFv-ILs-DTXp3 in SUM-149 xenograft model. FIG. 7C is a graph showing antitumor efficacy 40scFv-ILs-DTXp3 in MDA-MB-436 xenograft model. FIG. 7D is a graph showing the tolerability of 40scFv-ILs-DTXp3 in SUM-159 xenograft model. FIG. 7E is a graph showing the tolerability of 40scFv-ILs-DTXp3 in SUM-149 xenograft model. FIG. 7F is a graph showing the tolerability of 40scFv-ILs-DTXp3 in MDA-MB-436 xenograft model.

### Example 6: In Vivo Antitumor Efficacy of EphA2 Targeted DTXp3 Loaded Nanotherapeutic in Multiple Patient-Derived and Cell-Derived Xenograft Models in Mice

Antitumor efficacy of EphA2 targeted DTXp3 loaded nanotherapeutic was studied in 29 xenograft models at the dose of 50 mg/kg weekly for four weeks. Patient derived models were obtained from collaborators such as RPCI and Texas Tech University or from vendors such as Jacks Lab. Additionally, in 21 models EphA2-targeted-ILs-DTXp3 at 50 mg/kg were studied in comparison with free docetaxel at equitoxic or supra-toxic dose of 10 mg/kg.

The animal weight and tumor size were monitored twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume=[(length)×(width)²]/2

Maximum tumor regression was calculated according the following formulas where TV is tumor volume:

Max tumor regression=[(minimum TV−TV at day 0)/TV at day 0]×100

As shown in FIGS. 17A, EphA2 targeted DTXp3 loaded nanotherapeutic leads to tumor regression of more than 50% of tested models. Complete tumor regression (−100%) was seen in seen in 95/250 (39%) of tested animals, partial tumor regression (−30% and more) was seen in 112/250 (44.8%) and no tumor regression was only seen in 43/250 (18%). As shown in FIG. 17B, in 10/21 models (48%) EphA2-targeted-ILs-DTXp3 was significantly superior to free docetaxel. In the rest of the models EphA2-targeted-ILs-DTXp3 were similar to free docetaxel. There was no model in which free docetaxel led to significantly more tumor regression compared to EphA2-targeted-I Ls-DTXp3.

### Example 7: In Vivo Antitumor Efficacy of 40scFv-ILs-DTXp3 vs Non-Targeted NT-LS-DTXp3 in Triple Negative Breast Cancer and 46scFv-ILs-DTXp3 vs Non-Targeted NT-LS-DTXp3 in Gastric/Esophageal and Sarcoma Xenografts in Mice

Antitumor efficacy of 40scFv-ILs-DTXp3 was compared with it non-targeted version NT-LS-DTXp3 in the xenograft models of human triple negative breast cancer (TNBC) cell lines MDA-MB-436 and 46scFv-ILs-DTXp3 in the xenograft models of gastric/esophageal human cell lines OE-19, MKN-45 and OE-21. MDA-MB-436 was obtained from American Type Culture Collection (Rockville, Md.), OE-19, OE-21 were obtained from European Collection of Cell Cultures (Salisbury, UK) and MKN-45 was obtained from German collection of Microorganisms and cell cultures (Braunschweig, Germany).

MDA-MB-436 was propagated in L15 medium supplemented with 10% fetal calf serum, 50 U/ml penicillin G, and 50 μg/mL of streptomycin sulfate at 37° C., 5% CO2. OE-19, MKN-45 and OE-21 were propagated in RPM11640 medium supplemented with 10% fetal calf serum, 50 U/ml penicillin G, and 50 μg/mL of streptomycin sulfate at 37° C., 5% CO2.

NCR nu/nu homozygous athymic male nude mice (4-5 week old, weight at least 16 g) were obtained from Taconic. For MDA-MB-436, the mice were inoculated orthotopically into the mammary gland-right thoracic fat pad with 0.1 mL of the suspension containing 10×106 cells suspended in PBS supplemented with 30% growth factor reduced Matrigel® Matrix (Corning, cat. #354230). For the gastic/esophageal and sarcoma models, mice were inoculated ectopically into the flank with 0.2 mL of the suspension containing 5×10⁶ cells, 5×10⁶ cells, 5×10⁶ and 6×10⁶ cells for OE-19, OE-21, MKN-45 and SKIMS-1 respectively. For all the models cells were suspended in PBS. When tumors achieved the size between 150 mm³ and 350 mm³ the animals were assigned to the treatment groups according to the following method. The animals were ranked according to the tumor size, and divided into 3 categories of decreasing tumor size. Treatment groups of 5-8 animals/group were formed by randomly selecting one animal from each size category, so that in each treatment group all tumor sizes were equally represented.

For MDA-MB-436, the following treatment arms have been formed: 1) Control (HEPES-buffered saline pH 6.5); 2) NT-LS-DTXp3 at dose 50 mg/kg per injection; 3) 40scFv-ILs-DTXp3 at dose 50 mg/kg per injection. The animals received four tail vein injections, at the intervals of 7 days.

For model MDA-MB-436, The EphA2-ILs-DTX immunoliposome was 40scFv-ILs-DTXp3, with a lipid portion composed of egg sphingomyelin, cholesterol (3:2 molar ratio) and 8 mol % PEG-DSG, containing entrapped 1.1N TEA-SOS, prepared and loaded with Compound 3 at the drug/lipid ratio of 450 docetaxel equivalents/mole phospholipid as described in Example 1, and the scFv of SEQ ID NO:40 attached to the outside of the liposome.

Since gastric models are more sensitive we used a lower dose. For OE-19, MKN-45 and OE-21, the following treatment arms have been formed: 1) Control (HEPES-buffered saline pH 6.5); 2) NT-LS-DTXp3 at dose 25 mg/kg per injection; 3) 46scFv-ILs-DTXp3 (46scFv-ILs-DTXp3) at dose 25 mg/kg per injection.

Sarcoma model was known to be resistant to DTX and tested treated with the higher dose. The following treatment arms have been formed: 1) Control (HEPES-buffered saline pH 6.5); 2) NT-LS-DTXp3 at dose 50 mg/kg per injection; 3) 46scFv-ILs-DTXp3 (46scFv-ILs-DTXp3) at dose 50 mg/kg per injection.

The animal weight and tumor size were monitored once or twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume=[(length)×(width)²]/2

To assess treatment-related toxicity, the animals were also weighted twice weekly. When the tumors in the group reached 10% of the mouse body weight, the animals in the group were euthanized.
For MDA-MB-436, Average tumor volumes across the groups were plotted together and compared over time.
For OE-19, MKN-45 and OE-21, maximum response and time to regrowth were calculated according the following formulas where TV is tumor volume:

Max Response=[(minimum TV−TV at day 0)/TV at day 0]×100

Time to regrowth=time for TV to grow 4 times TV at day 0

FIG. 8A shows that 40scFv-ILs-DTXp3 has a significantly stronger antitumor efficacy comparing to non-targeted version NT-LS-DTXp3 in MDA-MB-436 xenograft models.

The treatment related toxicity was assessed by the dynamics of animals' body weight (FIG. 8B). Neither group revealed any significant toxicity. The weight of the animals in all treated groups was comparable to the control group and was consistently increasing.

FIG. 8C, FIG. 8D and FIG. 8E shows that 46scFv-ILs-DTXp3 has a significantly stronger antitumor efficacy comparing to non-targeted version NT-LS-DTXp3 in OE-19, MKN-45 and OE-21 xenograft models respectively. 46scFv-ILs-DTXp3 shows significantly higher response and/or a significant increase in time to growth in most models (FIG. 8F).

FIG. 8A is a graph showing the antitumor efficacy of NT-LS-DTXp3 compared to 40scFv-ILs-DTXp3 in MDA-MB-436 xenograft model. FIG. 8B is a graph showing the tolerability of NT-LS-DTXp3 vs 40scFv-ILs-DTXp3 in MDA-MB-436 xenograft model. FIG. 8C is a graph showing the antitumor efficacy of NT-LS-DTXp3 vs 46scFv-ILs-DTXp3 in OE-19 xenograft model. FIG. 8D is a graph showing the antitumor efficacy of NT-LS-DTXp3 vs 46scFv-ILs-DTXp3 in MKN-45 xenograft model. FIG. 8E is a graph showing the antitumor efficacy of NT-LS-DTXp3 vs 46scFv-ILs-DTXp3 in OE-21 xenograft model. FIG. 8F is a graph showing the antitumor efficacy of NT-LS-DTXp3 vs 46scFv-ILs-DTXp3 in OE-19, 0E-21 and MKN-45 xenograft models. FIG. 8G is a graph showing the antitumor efficacy of NT-LS-DTXp3 vs 46scFv-ILs-DTXp3 in SK-LMS-1 xenograft model.

### Example 8: In Vivo Antitumor Efficacy of 41scFv-ILs-DTXp3 in Non-Small Cell Lung Cancer Xenograft Model in Mice

Antitumor efficacy of 41scFv-ILs-DTXp3 was studied in a xenograft model of human non-small cell lung cancer cell line. A549 was obtained from American Type Culture Collection (Rockville, Md.) and propagated in RPMI1640 medium supplemented with 10% fetal calf serum, 50 U/ml penicillin G, and 50 mg/mL of streptomycin sulfate at 37° C., 5% CO2.

NCR nu/nu homozygous athymic male nude mice (5-6 week old) were obtained from Charles River. The mice were inoculated ectopically into the flank with 0.2 mL of the suspension containing 5×10⁶ cells suspended in PBS. When tumors achieved the size between 150 mm3 and 200 mm3 the animals were assigned to the treatment groups according to the following method. The animals were ranked according to the tumor size, and divided into 6 categories of decreasing tumor size. Treatment groups of 8 animals/group were formed by randomly selecting one animal from each size category, so that in each treatment group all tumor sizes were equally represented. The following treatment arms have been formed 1) Control (HEPES-buffered saline pH 6.5); 2) Docetaxel at dose 10 mg/kg per injection; 3) 40scFv-ILs-DTXp3 at dose 50 mg/kg per injection.

The animals received four tail vein injections, at the intervals of 7 days. The EphA2-ILs-DTX immunoliposome was 41scFv-ILs-DTXp3, with a lipid portion composed of egg sphingomyelin, cholesterol (3:2 molar ratio) and 5 mol % PEG-DSG, containing entrapped 1.1N TEA-SOS, prepared and loaded with Compound 3 at the drug/lipid ratio of 315 docetaxel equivalents/mole phospholipid as described in Example 1, and the scFv of SEQ ID NO:41 attached to the outside of the liposome.

Liposomes were prepared according to Example 1 version 40scFv-ILs-DTXp3. 1.Injection concentrated Docetaxel (Accord Healthcare Inc., 20 mg/ml alcohol solution) was dissolved prior administration in PBS to 1 mg/ml.

Tumor size was monitored twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume=[(length)×(width)²]/2

Average tumor volumes across the groups were plotted together and compared over time.

As shown in in FIG. 9, 41scFv-ILs-DTXp3 has a significantly stronger antitumor efficacy comparing to the equal toxic dose of Docetaxel in A549 model. FIG. 9 is a graph showing the antitumor efficacy 41scFv-ILs-DTXp3 in A549 xenograft model.

### Example 9: In Vivo Antitumor Efficacy of 41scFv-ILs-DTXp3 in Prostate and Ovarian Cancer Xenograft Model in Mice

Antitumor efficacy of 41scFv-ILs-DTXp3 was studied in a xenograft model of human prostate and ovarian cancer cell lines. DU-145 was obtained from American Type Culture Collection (Rockville, Md.), and OVCAR-8 was obtained from the National Cancer Institute (Bethesda, Md.). Both cell lines propagated in RPMI medium supplemented with 10% fetal calf serum, 50 U/ml penicillin G, and 50 μg/ml of streptomycin sulfate at 37° C., 5% CO2.

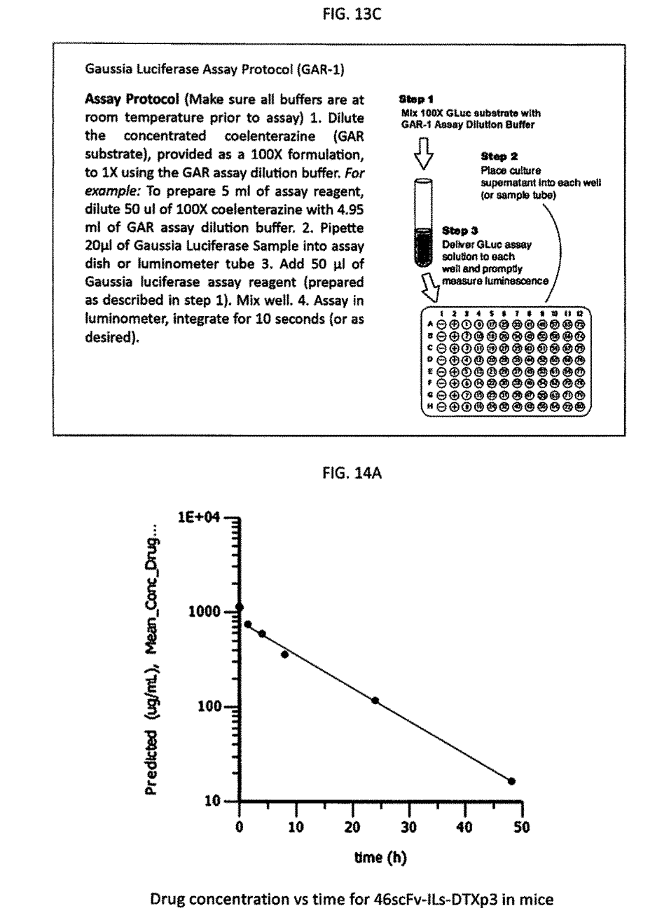

In order to enable orthotopic monitoring of OVCAR-8 cells in the peritoneal cavity, OVCAR-8 cells were infected with Gaussia luciferase (GLUC) expressing lentiviral supernatant obtained from Targeting Systems (El Cajon, Calif.) (FIG. 13B). Briefly, FIG. 13B shows a diagram of the commonly used virus production and infection protocol. Upon infection (MOI˜10) and Puromycin selection (2 weeks, 1 ug/ml) of the wished cell line, bioluminescence assay was used to evaluate GLUC expression in the media. The principles of the measurement for secreted GLUC was previously described in Chung et al PLOS One 2009, Dec. 15; 4(12):e8316. However, adapted and modified protocol from Targeting Systems (FIG. 13C) was used and is described in FIG. 2. Briefly, samples either plasma or media was transferred to a 96-well plate. Gluc activity was measured using a plate luminometer (MLX luminometer, Dynex technologies, Chantilly, Va.). The luminometer was set to automatically inject 100 ml of 100 mM coelenterazine (CTZ, Nanolight, Pinetop, Ariz.) in PBS and photon counts were acquired for 10 sec. The assay was used with supernatant for in vitro assessment of the cells, and was used on plasma for in vivo monitoring of tumor burden. In summary, blood samples were collected weekly through saphenous bleed, blood was centrifuged and plasma was extracted. Bioluminescence assay as described above was used on the same day as collection to ensure signal stability.

For OVCAR-8 experiment, NCR nu/nu homozygous athymic female nude mice (5-6 week old) were obtained from Charles River. The mice were inoculated intraperitoneally with 0.3 ml of the suspension containing 0.5×10⁶ cells suspended in RPMI. Animals were bled weekly for GLUC assessment. When tumor burden reached 20 ng/ml of plasma GLUC levels, animals were assigned to the treatment groups according to the following method. Given the variability is disease progression, animals were assigned to various groups in rolling fashion and randomized to either control or 46svFV-ILs-DTXp3 group. The following treatment arms have been formed 1) Saline (HEPES-buffered saline pH 6.5); 2) 46scFv-ILs-DTXp3 at dose of 20 mg/kg per injection (46scFv-ILs-DTXp3 20mpk).

As shown in FIG. 13A, 46scFv-ILs-DTXp3 at 20 mg/kg induced inhibition of tumor growth persistent throughout the duration of treatment.

For DU-145 experiment, NCR nu/nu homozygous athymic female nude mice (5-6 week old) were obtained from Charles River. The mice were inoculated ectopically into the flank with 0.2 mL of the suspension containing 3.3×106 cells suspended in RPMI supplemented with 50% growth factor reduced Matrigel® Matrix (Corning, cat. #354230). When tumors achieved the size between 150 mm3 and 200 mm3 the animals were assigned to the treatment groups according to the following method. The animals were ranked according to the tumor size, and divided into 4 categories of decreasing tumor size. Treatment groups of 6 animals/group were formed by randomly selecting one animal from each size category, so that in each treatment group all tumor sizes were equally represented. The following treatment arms have been formed 1) Saline (HEPES-buffered saline pH 6.5); 2) Docetaxel at dose 10 mg/kg per injection (DTX 10mpk); 3) 46scFv-ILs-DTXp3 at dose of 25 mg/kg per injection (46scFv-ILs-DTXp3 25mpk) 4) 46scFv-ILs-DTXp3 at dose 50 mg/kg per injection (46scFv-ILs-DTXp3 50mpk).

The animals received four tail vein injections, at the intervals of 7 days. Injection concentrated docetaxel (Accord Healthcare Inc., 20 mg/ml alcohol solution) was dissolved prior administration in PBS to 1 mg/ml. Tumor size was monitored once to twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume (TV)=[(length)×(width)²]/2

Maximum response was calculated using the following formula where TV is tumor volume:

Max Response=[(minimum TV−TV at day 0)/TV at day 0]×100

Single tumor volumes across the groups were plotted together and compared over time.

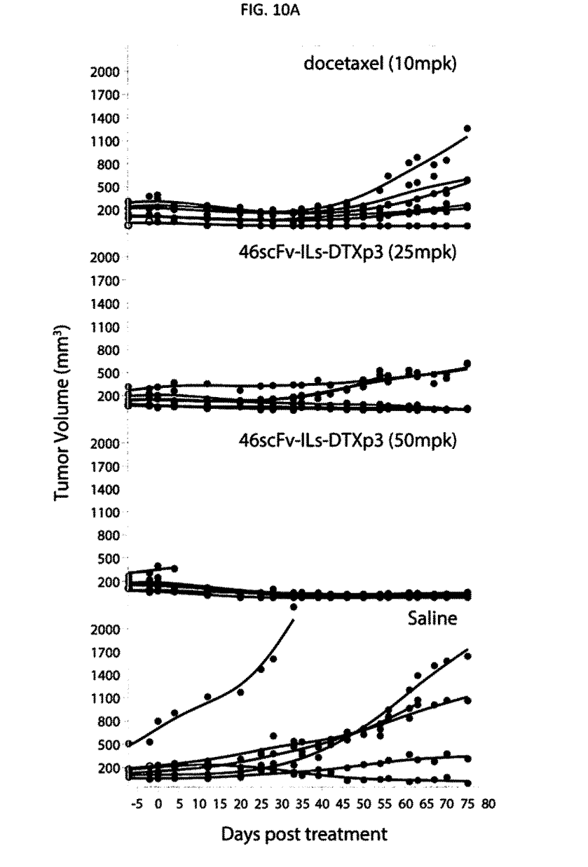

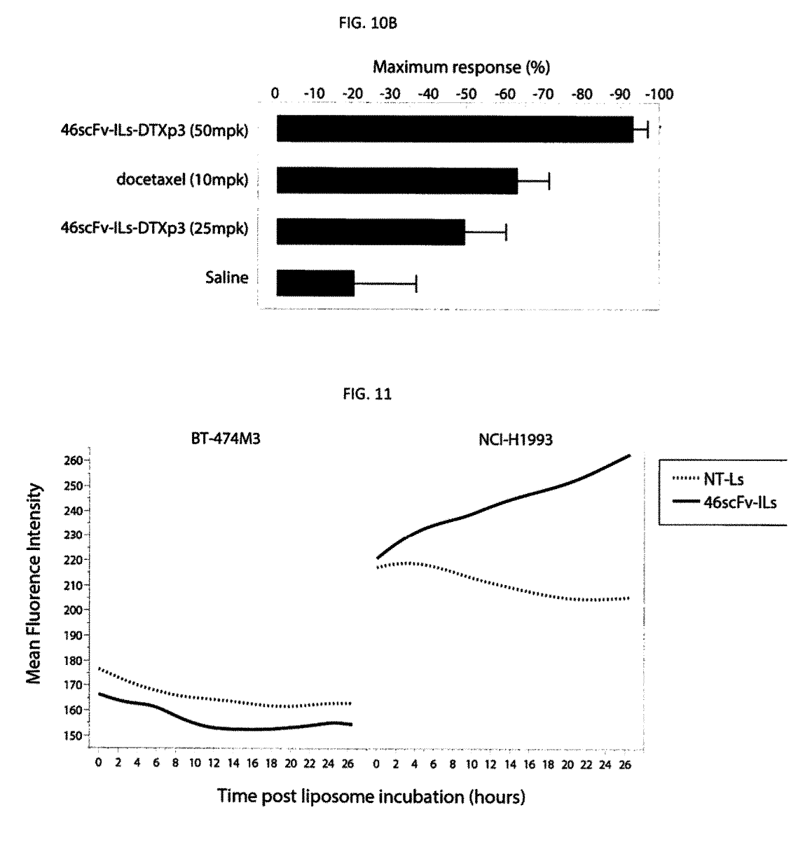

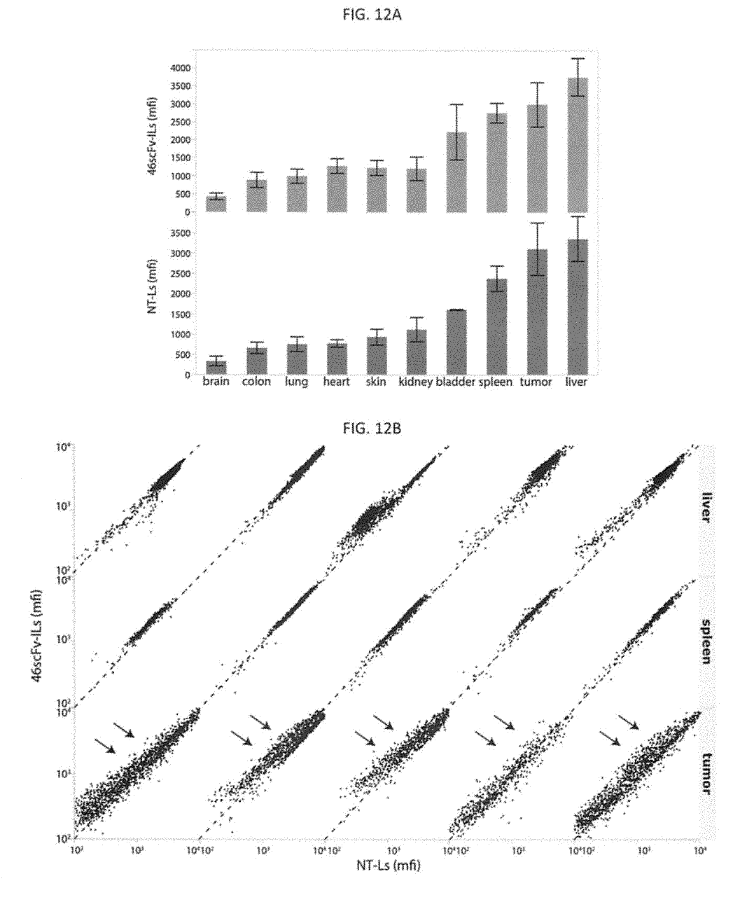

As shown in in FIGS. 10A and 10B, 46scFv-ILs-DTXp3 at 50 mg/kg of bw has a significantly stronger antitumor efficacy comparing to the equitoxic dose 10 mg/kg of free Docetaxel in DU-145 model. FIG. 10A is a graph showing antitumor efficacy 46scFv-ILs-DTXp3 in DU-145 xenograft model. FIG. 10B is a graph showing antitumor efficacy 46scFv-ILs-DTXp3 in DU-145 xenograft model.

### Example 10: In Vivo Tolerability of 41scFv-ILs-DTXp3 and Free Docetaxel in Swiss Webster Mice

This example describes In vivo tolerability of EphA2 targeted docetaxel nanoliposome (46scFv-lLs-DTXp3) and of free docetaxel was studied in NCR nu/nu mice.

Swiss Webster homozygous female mice (5-6 week old) were obtained from Charles River Laboratory. The animals received tail vein injections, at the intervals of 7 days of tested compound at tested dose (Table 5: Experimental design). 41scFv-ILs-DTXp3 doses are expressed at equivalent docetaxel weight.

TABLE 5
Experimental Design

| # of | Treatment | Dose |
|---|---|---|
| mice | (Compound, route) | (mg/kg bw) |
| 3 | 41scFv-ILs-DTXp3, IV | 93, 107, 123, 167 |
| 3 | DTX, IV | 20, 23, 26 |

The EphA2-ILs-DTX immunoliposome was 41scFv-ILs-DTXp3, with a lipid portion composed of egg sphingomyelin, cholesterol (3:2 molar ratio) and 6 mol % PEG-DSG, containing entrapped 1.1N TEA-SOS, prepared and loaded with Compound 3 at the drug/lipid ratio of 315 docetaxel equivalents/mole phospholipid as described in Example 1, and the scFv of SEQ ID NO:41 attached to the outside of the liposome. The animals received tail vein injection of 41scFv-ILs-DTXp3 or free docetaxel at specific doses. Animal weight and performance status score were monitored based on clinical observation of the mice three to four times a week (clinical observations table). Animals that experience severe toxicity are euthanized according to IACUC protocol. If more than 30% of the mice experience severe toxicity the tested dose is deemed not tolerated.

TABLE 6
Clinical observation.

| Parameter | Mild | Moderate | Severe |
|---|---|---|---|
| Body | Weight loss <10% | Weight loss 10-20% | Weight loss >20% of |
| condition | of the bodyweight | of the bodyweight | the bodyweight |
| | Food and water | Food and water | Food and water |
| | consumption | consumption appear | consumption appear |
| | appear untouched | untouched for up to | untouched for up to |
| | for up to 72 h | 72 h | 72 h. |
| External | Partial | Piloerection | Sunken abdomen, |
| body | piloerection | Hunched most of | muscle wasting and |
| appearance | Transiently | the time | poor hair quality |
| (Posture) | hunched | | Piloerection |
| | | | Persistently hunched |
| | | | Tremor or shivering |
| Clinical | Lethargic, mild | Skin: pale ears, | Skin: pruritus, |
| signs | salivation | nose, eyes, | alopecia, urticaria, |
| | | rough haircoat, | severe site |
| | | itching, mild local | inflammation, |
| | | site inflammation | erosions, necrosis, |
| | | Respiratory: visible | plaques, petechial |
| | | intermittent | bleedings |
| | | dyspnoea, sneezing, | Respiratory: labored |
| | | mild nasal discharge | breathing, coughing, |
| | | Alimentary: | cyanosis, copious |
| | | salivation, anorexia, | nasal discharge |
| | | mild diarrhea, | Alimentary: |
| | | Nervous system: | hypersalivation, |
| | | ataxia, abnormal | anorexia, persistent |
| | | gait & posture | diarrhea, icteric |
| | | Other: mild | mucous membranes |
| | | lameness, ocular | Nervous system: |
| | | discharge, mild | circling, abnormal |
| | | discomfort | gait & posture, |
| | | | convulsion, paralysis |
| | | | Other: serious |
| | | | lameness, necrotic |
| | | | tail, fever, obvious |
| | | | pain |

After three doses of 41scFv-ILs-DTXp3 at 167 mpk, all animals of the group had dry scaly skin with the appearance of pre-ulceration redness requiring treatment termination and animal euthanasia. 41scFv-ILs-DTXp3 at 123mpk showed milder skin drying and scaling which did not require euthanasia and was treated with hydrating crème. Given this finding the maximum tolerated dose for weekly treatment with 41scFv-ILs-DTXp3 in Swiss Webster mice is identified 123 mpk. For both groups, body weight loss was mild <10%.

After three doses of free docetaxel at the dose 23 and 26 mpk, all animals of the group had weight loss ranging from 10% to 15% and signs of hypersensitivity (vocal at touch) and evidence of local toxicity at site of injection (puffy tail). These signs were deemed severe requiring delaying or interrupting treatment which is considered a dose limiting toxicity. Docetaxel at 20 mpk was well tolerated and did not lead to any detectable weight loss or any toxicity sign. Given these findings the maximum tolerated dose for weekly treatment with free docetaxel in Swiss Webster mice is 20 mpk.

FIG. 6A is a graph showing the tolerability of 41scFv-ILs-DTXp3 in Swiss Webster mice. FIG. 6B is a graph showing the tolerability of free docetaxel in Swiss Webster mice.

### Example 11: Synthesis of Docetaxel Prodrugs

Docetaxel prodrugs of formula (I) can be prepared by a various reaction methods, including the reaction scheme shown in FIG. 2A. Representative synthetic examples of two compounds are provided below. These or other docetaxel prodrugs, and various pharmaceutically acceptable salts thereof, can be prepared by various suitable synthetic methods. The Table in FIG. 2B provide a representative list of examples of certain docetaxel prodrugs.

### Example 11A: Synthesis of 2′-O-(4-Diethylamino butanoyl) DTX Hydrochloride (Compound 3

Docetaxel (DTX) (0.25 g, 0.31 mmol), 4-diethylamino butyric acid hydrochloride (0.12 g, 0.62 mmol), EDAC.HCl (0.12 g, 0.62 mmol), and DMAP (0.08 g, 0.62 mmol) were all weighed into a 15 mL vial under Ar. To this 6 ml of anhydrous DCM was added at rt under Ar and stirred at rt for 18 h. HPLC after 18 h stirring shows 43% product with 57% DTX remaining unreacted.

Additional amount of 4-diethylamino butyric acid hydrochloride (0.15 g, 0.77 mmol) was added and stirring continued for additional 24 h. HPLC shows 91% product with 8% DTX remaining unreacted. The reaction was stopped and directly loaded on a 12 g cartridge and FCC was performed using 3-12% MeOH/CHCl3. Fractions 30-49 were pooled together, 0.5 mL of 0.05N HCl in 2-propanol was added and evaporated under 30 ²C to give 0.19 g of a white solid (63% yield).

HNMR: δ=8.11 (d, 2H), 7.65-7.57 (m, 1H), 7.56-7.46 (m, 2H), 7.45-7.29 (m, 5H), 6.29-6.12 (m, 1H), 5.99 (d, 1H), 5.68 (d, 1H), 5.58-5.40 (m, 1H), 5.31 (d, 1H), 5.24 (s, 1H), 4.96 (d, 1H), 4.38-4.08 (m, 5H), 3.91 (d, 1H), 3.20-2.30 (m, 3H), 2.70-2.52 (m, 2H), 2.50-2.42 (m, 2H), 2.25-2.05 (m, 3H), 1.94 (s, 3H), 1.92-1.78 (m, 2H), 1.75 (s, 3H), 1.50-1.35 (m, 9H), 1.32 (s, 9H), 1.30-1.20 (m, 6H), 1.12 (s, 3H) ppm. ppm. MS (ESI) m/z: 949.4 [M]+

### Example 11B: Synthesis of 2′-O-[4-(N,N-Dimethylamino) butanoyl] DTX Hydrochloride (Compound 4)

Docetaxel (DTX) (0.28 g, 0.34 mmol), N,N-dimethylaminobutyric acid hydrochloride (0.07 g, 0.43 mmol), EDAC.HCl (0.13 g, 0.69 mmol) and DMAP (0.05 g, 0.41 mmol) were all weighed into a 15 mL vial under Ar. To this 7 mL of anhydrous DCM was added and the mixture was stirred at rt for 18 h. HPLC after 18 h shows 94% product with 3% byproduct and 3% of DTX remaining. The reaction residue was directly loaded on a 12 g cartridge and FCC was performed using 5-50% 2-propanol/CHCl3. Fractions 22-41 were pooled together, 0.5 ml of 0.05N HCl in 2-propanol was added and evaporated under 40° C. to give a white solid weighing 0.16 g (48% yield). Relatively low yield despite good conversion of DTX to product was presumably due to the poor solubility of the product in 2-propanol.

1H NMR (300 MHz, CDCl3+ (CD3)2SO): δ=8.02 (d, 2H), 7.60-7.42 (m, 6H), 7.38-7.25 (m, 4H), 7.24-7.12 (m, 1H), 6.92 (d, 1H), 6.40-5.88 (m, 1H), 5.53 (d, 1H), 5.35-5.21 (m, 1H), 5.20-5.08 (m, 2H), 4.85 (d, 1H), 4.74 (d, 1H), 4.26 (s, 1H), 4.13 (dd, 3H), 3.74 (d, 1H), 3.56 (s, 1H), 3.16-2.74 (m, 3H), 2.64-2.48 (m, 7H), 2.49-2.00 (m, 5H), 1.84-1.68 (m, 4H), 1.62 (m, 3H), 1.30 (s, 9H), 1.07 (s, 3H), 1.02 (s, 3H) ppm. MS (ESI) m/z: 921.4 [M]+

### Example 12. Procedure for Hydrolysis in Buffer

A volume of a 10 mg/mL solution of drug in DMSO as needed to provide the desired concentration (typically 16 p1 to yield a 80 μg/mL solution) is placed in a glass test tube or 4 mL vial. Additional DMSO may also be added (e.g. 64 μL when using 164 of drug solution) to yield a final total DMSO concentration of 4%. The DMSO solutions are mixed by brief vortexing, and then 2 mL (20 mM) HEPES buffer for pH 7.5 and 2 mL of (20 mM) phosphate buffer for pH 2.5 is added and the mixture is vortexed again. The initial pH may be adjusted by addition of HCl or NaOH. The use of 20 mM buffer was found to provide better pH control and to avoid pH drift during incubation.

Then 100 μLaliquots of the buffer solution of drug are transferred into HPLC vials and incubated in a 37° C. water bath. The remaining solution is also incubated at 37° C. in 4 mL vials for monitoring of pH.

At each time point 900 μL of 0.1% trifluoroacetic acid (TFA) in acetonitrile (ACN) is added to the HPLC vial and the contents are vortexed. The vials are then placed in an autosampler rack at 4° C. for HPLC analysis.

Time zero data points are typically obtained from a solution of 4 μL DMSO stock in 5 mL of 0.1% TFA/ACN (8 μg/mL).

HPLC analysis is performed on a SYNERGI 4 micron Polar RP-80A, 250×4.6 mm column, using a flow rate of 1 mL/min, a 50 μL injection volume, column temperature of 25° C. and with UV detection at 227 nm. Most compounds are analyzed using a 13 min gradient (Method A) from 30 to 66% acetonitrile in aqueous 0.1% TFA, followed by a 1 min gradient back to 30% and a hold at 30% for 6 minutes. If the retention time is too long for this method, a 20 min gradient (Method B) of 30 to 90% acetonitrile, followed by a 1 min return to 30% and held for 9 min at 30% is employed.

The extent of hydrolysis is reported in Table 7 below as the % DTX formation based on relative peak areas.

TABLE 7

| | | Percentage of Hydrolysis at | | | | | |
|---|---|---|---|---|---|---|---|
| | | Different Time Points (h) | | | | | |
| Compound | pH | 0 | 2.5 | 5 | 24 | 48 | 96 |
| Compound 1 | 2.5 | 0.00 | n/a | 0.00 | 0.00 | 0.00 | 0 |
| | 7.5 | 0.00 | n/a | 23.41 | 51.38 | 69.00 | 88 |
| Compound 3 | 2.5 | 0.00 | n/a | 0.00 | 0.00 | 0.00 | 0 |
| | 7.5 | 0.00 | n/a | 45.76 | 100.00 | 100.00 | 100 |
| Compound 4 | 2.5 | 0.00 | n/a | 0.00 | 0.00 | 0.00 | 0 |
| | 7.5 | 0.00 | n/a | 76.07 | 100.00 | 100.00 | 100 |

### Example 13. Procedure for Hydrolysis in Buffered Plasma

Human plasma (HP 1055 from Valley Biomedical Inc, Winchester, Va.; pooled human plasma preserved with Na citrate) is centrifuged to remove precipitate. To 1.5 mL centrifuge tubes is added 0.9 mL of plasma and 40-50 μL of pH 7.5, 0.9 M HEPES buffer (final concentration of 40-50 mM and pH of 7.5). This is mixed by inversion, and then the tubes are warmed to 37° C. Then 7.2 μL of a 10 mg/mL DMSO solution of drug is added (80 ug/mL final concentration) and the contents mixed by inversion. The solution in plasma is then aliquoted into 1.5 mL centrifuge tubes and placed in a 37° C. bath. At each time point 900 μL of 0.1% trifluoroacetic acid (TFA) in acetonitrile (ACN) is added to the tubes. The contents are vortex mixed and then centrifuged for 5 min at 13,000 rpm. The supernatants are analyzed by HPLC as described under Procedure for Hydrolysis in Buffer. Time zero data points are obtained from a solution of 4 μL DMSO stock in 5 ml of 0.1% TFA/ACN (8 μg/mL). The results are typically compared to those obtained for 80 μg/mL in 50 mM Hepes buffer (pH 7.5) with 4% DMSO, using the Procedure for Hydrolysis in Buffer.

### Example 14. Pharmacokinetics of EphA2-Targeted Docetaxel-Generating Liposomes in Mice

The care and treatment of experimental animals was in accordance with Institutional Animal Care and Use Committee (IACUC) guidelines, Protocol MAP004. Female Swiss Webster mice aged 6-8 weeks were obtained from Charles River Laboratories (Wilmington, Mass.). Liposomes prepared in Example 1 were used for pharmacokinetics study. For each liposome formulation, a group of three mice was dosed with tritium-labeled liposomes at given dose (2-50 mg/kg). Blood samples (30-60 μL) were collected into lithium heparin coated tubes through saphenous vein at time points specified as: 5 min, 1.5 h, 4 h, 8 h, 24 h. At 48 h time point, approximately 400 μL blood was collected through terminal cardiac bleeding. Blood samples were immediately centrifuged (within 10 min) at 12,000 rpm for 5 min at 4° C. Plasma samples were then removed and stabilized with 200 mM pH 2.5 phosphate buffer with a dilution factor no less than 3. The stabilized samples were split for radioactivity counting and drug concentration measurement. The concentration over time data for lipids, Compound 3, and drug lipid ratio were analyzed by Phoenix software using NCA model. The PK results of different liposome formulations are summarized in Table 8. Formulations of the liposome Ls-DTXp3 at different drug to phospholipid (D/L) ratios, and immunoliposome 46scFv-ILs-DTXp3 at different % PEG-DSG were evaluated.

TABLE 8
Pharmacokinetics of Ls-DTXp3 and scFv-ILs-DTXp3

| | | % | Release | | |
|---|---|---|---|---|---|
| | D/L | PEG- | Rate | Lipids | Drug |
| Formulation | (g/mol) | DSG | t_{1/2} (h) | t_{1/2} (h) | t_{1/2} (h) |
| Ls-DTXp3-150 | 150 | 8 | 46.49 | 10.16 | 8.30 |
| Ls-DTXp3-300 | 300 | 8 | 78.13 | 10.60 | 9.67 |
| Ls-DTXp3-450 | 450 | 8 | 100.70 | 9.83 | 8.97 |
| Ls-DTXp3-600 | 600 | 8 | 153.00 | 10.82 | 10.10 |
| 46scFv-ILs-DTXp3-300 | 300 | 6 | 108.95 | 9.49 | 8.59 |

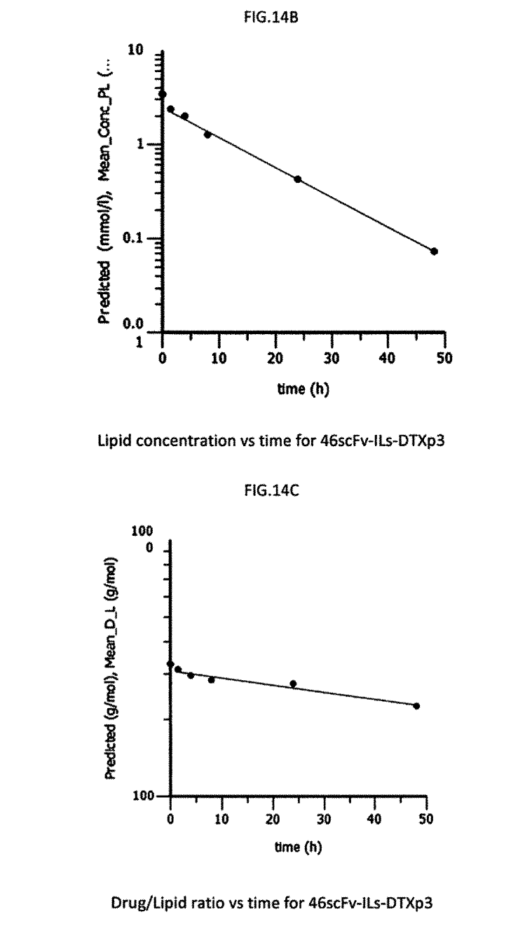

Pharmacokinetics profiles of 46scFv-ILs-DTXp3-300 are shown in FIG. 14A, 14B and 14C for drug vs time, lipid vs time, and Drug/lipid ratio vs time respectively. The experimental data are fitted with non-compartmental analysis shown as solid line.

### Example 15. Hematologic Toxicity and Coagulation Parameters Induced by Chronic Treatment with Free Docetaxel vs. 46scFv-ILs-DTXp3 in Rats

The objectives of this study were to evaluate taxane induced hematologic toxicity resulting from 46scFv-ILs-DTXp3 and free docetaxel exposure in male rats when given by once weekly intravenous infusion (4 doses). The study design was as follows:

TABLE 9

| Test Material | Dose mpk | Regimen | n |
|---|---|---|---|
| Vehicle Control | 0* | Weekly doses × 4 | 6 |
| (46scFv-ILs)* | | | |
| Free docetaxel | 10 | Weekly doses × 4 | 6 |
| 46scFv-ILs-DTXp3 | 10 | Weekly doses × 4 | 6 |
| 46scFv-ILs-DTXp3 | 20 | Weekly doses × 4 | 6 |
| 46scFv-ILs-DTXp3 | 40 | Weekly doses × 4 | 6 |

NOTE:
Vehicle control was a non drug-loaded 46scFv-ILs. The dose administered was based on phospholipid dose equivalent to the highest dose of 46scFv-ILs-DTXp3administered (40 mg/kg).

The following parameters and end points were evaluated in this study: clinical signs, body weights, food consumption, clinical pathology parameters (hematology, coagulation, clinical chemistry, and urinalysis), gross necropsy findings, and histopathologic examinations.

Blood was collected from a jugular vein (Day 18=Day 4 post 3^{rd} dose) or the abdominal aorta after isoflurane anesthesia (study termination=Day 9 post 4th dose). On Day 18, only the hematology sample was collected for rats that weighed less than 350 g. After collection, samples were transferred to the appropriate laboratory for processing. Animals were fasted overnight before blood.

Blood samples were analyzed for the parameters specified: Red blood cell count, Hemoglobin Concentration, Hematocrit, Mean corpuscular volume, Red Blood Cell Distribution Width, Mean corpuscular hemoglobin concentration, Mean corpuscular hemoglobin, Reticulocyte count (absolute), Platelet count, White blood cell count, Neutrophil count (absolute), Lymphocyte count (absolute), Monocyte count (absolute), Eosinophil count (absolute), Basophil count (absolute), Large unstained cells.

Once weekly 10 minutes intravenous infusion free docetaxel 10mpk did induce pancytopenia with extensive decrease of all the components of the hematologic profile at day 4 post the 3^{rd} dose and partially persisted at 10 days post the 4th and final dose. Once weekly 10 minutes intravenous infusion free docetaxel of 46scFv-ILs-DTXp3 at doses of 10, 20, and 40 mg/kg/dose for a total of 4 doses in Sprague-Dawley rats led to significantly less hematologic toxicity with no detectable effects on red blood cells and variable effects on white blood cells. The effects of 46scFv-ILs-DTXp3 were only seen at the highest dose of 40 mpk at both analyzed time points (day 4 post 3^{rd} dose and day 10 post 4th dose).

TABLE 10
Hematologic toxicity at 4 days post the 3rd
dose of 46scFv-ILs-DTXp3 or free docetaxel

| | | 46scFv- | 46scFv- | 46scFv- | |
|---|---|---|---|---|---|
| | | ILs- | ILs- | ILs- | |
| | | DTXp3 | DTXp3 | DTXp3 | docetaxel |
| | Vehicle | 10 mpk | 20 mpk | 40 mpk | 10 mpk |
| | Control | % change | % change | % change | % change |
| RBC (10⁶/μL) | 8.850 | 0 | 0 | −9 | −23 |
| HGB (g/dL) | 16.96 | 0 | 0 | −8 | −19 |
| HCT (%) | 50.84 | 0 | 0 | −7 | −21 |
| RDW (%) | 12.80 | 0 | 0 | 0 | +30 |
| PLT (10³/μL) | 1279.2 | −23 | −31 | −32 | −53 |
| RETIC (10⁹/L) | 264.10 | −26 | −49 | −76 | −93 |
| WBC (10³/μL) | 14.684 | −30 | −11 | −30 | −81 |
| NEUT (10³/μL) | 1.356 | 0 | +118 | +57 | −56 |
| LYMPH | 12.396 | −31 | −24 | −38 | −82 |
| (10³/μL) | | | | | |
| MONO | 0.262 | 0 | +73 | 0 | −87 |
| (10³/μL) | | | | | |
| EOS (10³/μL) | 0.102 | 0 | −28 | −68 | −100 |
| BASO (10³/μL) | 0.338 | −72 | −77 | −82 | −96 |
| LUC (10³/μL) | 0.230 | −38 | −23 | −44 | −90 |

TABLE 11
Hematologic toxicity at 9 days post the 4th
dose of 46scFv-ILs-DTXp3 or free docetaxel

| | | 46scFv- | 46scFv- | 46scFv- | |
|---|---|---|---|---|---|
| | | ILs- | ILs- | ILs- | |
| | | DTXp3 | DTXp3 | DTXp3 | |
| | Vehicle | 10 mpk | 20mpk | 40mpk | docetaxel |
| | Control | % change | % change | % change | % change |
| RBC (10⁶/μL) | 7.728 | 0 | 0 | −27 | −39 |
| HGB (g/dL) | 14.25 | 0 | 0 | −27 | −31 |
| HCT (%) | 18.43 | 0 | 0 | 0 | +12 |
| RDW (%) | 13.42 | 0 | 0 | +42 | +113 |
| PLT (10³/μL) | 1231.0 | 0 | 0 | 0 | −42 |
| RETIC (10⁹/L) | 300.77 | 0 | 0 | 0 | +139 |
| WBC (10³/μL) | 11.208 | 0 | 0 | 0 | −59 |
| NEUT (10³/μL) | 0.962 | 0 | +120 | +106 | −48 |
| LYMPH | 9.785 | 0 | 0 | 0 | −62 |
| (10³/uL) | | | | | |
| MONO | 0.188 | 0 | +73 | +127 | 0 |
| (10³/μL) | | | | | |
| EOS (10³/μL) | 0.102 | 0 | 0 | −84 | −97 |
| BASO (10³/μL) | 0.055 | 0 | 0 | 0 | −73 |
| LUC (10³/μL) | 0.120 | 0 | 0 | 0 | 0 |

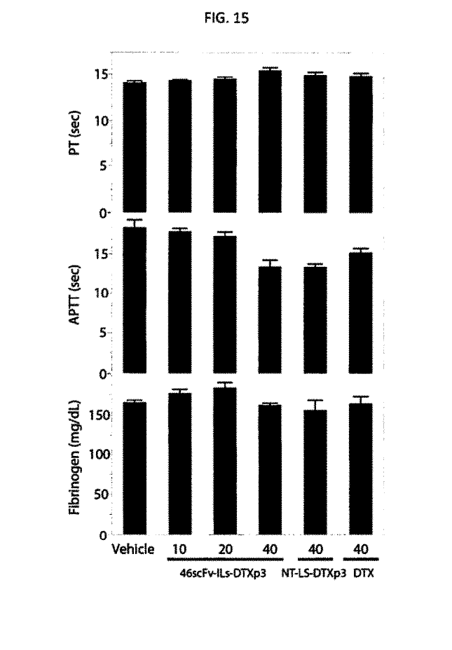

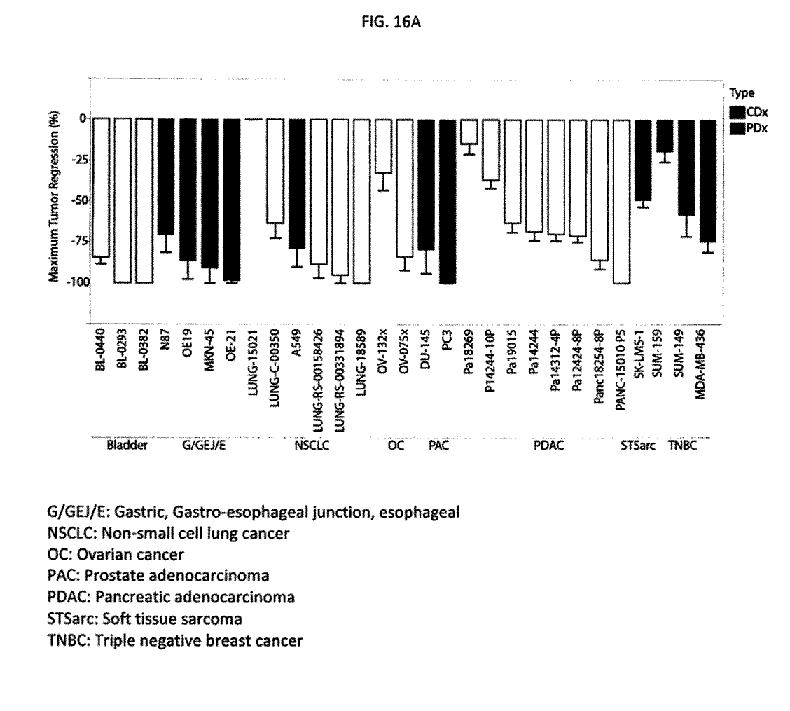

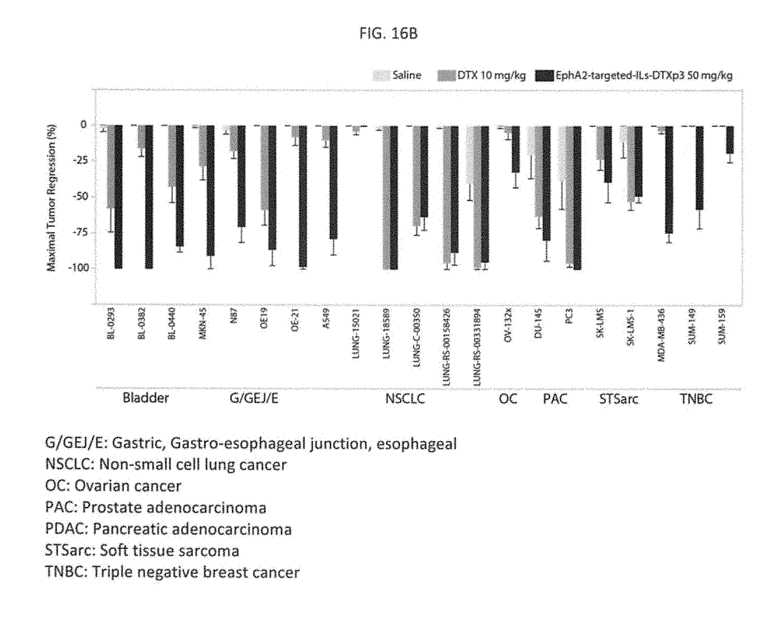

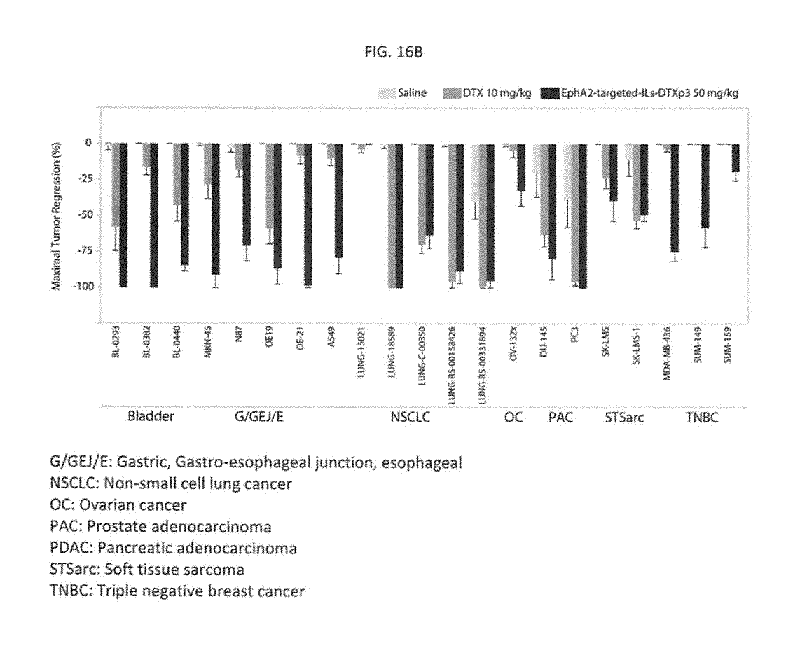

An additional group of Sprague Dawley rats treated with NT-LS-DTXp3 at 40 mg/kg weekly was used to test the potential presence of EphA2 targeting-mediated effects on coagulation. Blood samples collected at end of study from all the tested groups were analyzed for coagulation-related parameters including: Prothrombin time (PT), activated partial thromboplastin time (APTT) and fibrinogen levels. FIG. 15 shows PT, APTT and fribrinogen mean and standard error of mean for all the groups. The effects of 46scFv-ILs-DTXp3 or NT-Ls-DTXp3 were very similar to free docetaxel with relatively low effect on PT, APTT and fibrinogen.

### Example 16. A Novel EphA2-Targeted Docetaxel Antibody Directed Nanotherapeutic (ADN)

Taxanes are widely used to treat solid tumors either in the curative or palliative setting, in first or later lines of therapy. Analysis of docetaxel dose-response relationship strongly suggests that a higher dose would lead to a greater response, however a higher dose will also lead to higher toxicity. This is likely related to the lack of organ and cellular specificity of docetaxel leading to high exposures in normal tissues and the relatively short circulation half-life, which indirectly requires higher doses. With the goal of addressing the pharmacokinetic limitations of free docetaxel and the lack of cellular specificity, we developed a novel liposome delivering docetaxel targeted against Ephrin receptor A2 (EphA2) (46scFv-ILs-DTXp3), which is overexpressed in a wide range of tumors. 46scFv-ILs-DTXp3 provides sustained release of docetaxel following accumulation in solid tumors. Preclinical models have demonstrated that 46scFv-ILs-DTXp3 leverages tumor-specific accumulation through the enhanced permeability and retention effect, and cellular specificity through active targeting of EphA2 with specific scFv antibody fragments conjugated to the surface of the liposomes.

Pharmacokinetic and biodistribution studies were performed in mice and rats to compare 46scFv-ILs-DTXp3 to free docetaxel. Chronic tolerability studies were performed in rodent and non-rodent models with focus on overall animal health, as well as hematologic toxicities. Several cell-derived models of breast, lung and prostate xenografts were used to evaluate the differences between 46scFv-ILs-DTXp3 and free docetaxel.

In preclinical testing, 46scFv-ILs-DTXp3 had a significantly longer half-life than free docetaxel with prolonged exposure at the tumor site. In chronic tolerability studies, 46scFv-ILs-DTXp3 was found to be 6-7 times better tolerated than free docetaxel with a maximum tolerated dose of at least 120 mg/kg, compared to 20 mg/kg for free docetaxel and no detectable hematological toxicity. At equitoxic dosing, 46scFv-ILs-DTXp3 50 mg/kg showed greater activity than docetaxel 10 mg/kg in several breast, lung and prostate xenograft models.

In conclusion, we developed a novel EphA2 targeted docetaxel nanoliposome with prolonged circulation time and slow and sustained drug release kinetics to enable organ and cellular targeting. 46scFv-ILs-DTXp3 was able to overcome hematologic toxicities observed upon treatment with free docetaxel in rodent and non-rodent models. 46scFv-ILs-DTXp3 was also able to induce tumor regression or control tumor growth in several cell-derived xenograft models, and was found to be more active than free docetaxel in most models.

Sprague-Dawley Rats were given once weekly 10 minute intravenous infusions of 10, 20, and 40 mg/kg/dose 46scFv-ILs-DTXp3 or 10 mg/kg docetaxel for a total of four (4) doses. No significant effects were seen on coagulation parameters. Activated partial thromboplastin time (APTT) was shortened for rats that received 40 mg/kg 46scFv-ILs-DTXp3 and 10 mg/kg docetaxel at the end of the study (Day 30), compared to vehicle control. Prothrombin time (PT) was slightly increased in animals that received 46scFv-ILs-DTXp3 at 40 mg/kg/dose. Fibrinogen was not affected by these treatments. Dose-dependent changes in the erythrocyte and leukocyte parameters were observed, which correlated with the hematopoietic and lymphoid changes noted microscopically (data not shown). Contrary to 10 mg/kg docetaxel, there were no effects of 46scFv-ILs-DTXp3 at any dose level on neutrophil levels.

Once weekly 1 hour intravenous infusion of 1, 3, and 9 mg/kg/dose 46scFv-ILs-DTXp3 for a total of four (4) doses in Beagle dogs had no effect on hematology or any coagulation parameters.

46scFv-ILs-DTXp3 displayed a slow and sustained drug release that resulted in only low levels of bioavailable free docetaxel in the circulation, and elevated levels of active drug in the tumor. This targeted nanoformulation displayed a favorable toxicity profile, including minimal neutropenia, the dose-limiting toxicity of the current commercial polysorbate formulation of docetaxel. 46scFv-ILs-DTXp3 also demonstrated superior antitumor activity in multiple preclinical xenograft models compared to free docetaxel at or below equitoxic dosing.

### Example 17. Clinical Testing of EphA2-Targeted Docetaxel Liposome

A clinical study of EphA2-targeted docetaxel liposome is conducted to evaluate the activity of EphA2-targeted docetaxel liposome in patients with solid tumors. EphA2-targeted docetaxel liposome will be assessed as a monotherapy until a maximum tolerated dose (MTD) is established. EphA2-targeted docetaxel liposome will be administered by IV infusion over 90 minutes on the first day of each 21 day cycle.

### Inclusion Criteria:

To be eligible for inclusion in the study patients must have one of the following cancers, for which the patient has either received or been intolerant to all therapy known to confer clinical benefit
Urothelial carcinoma
Gastric/gastroesophageal junction/esophageal carcinoma (G/GEJ/E)
Squamous Cell Carcinoma of the Head and neck (SCCHN)
Ovarian cancer
Pancreatic ductal adenocarcinoma (PDAC)
Prostate adenocarcinoma (PAC)
Non-small cell lung cancer (NSCLC)
Small cell lung cancer (SCLC)
Triple negative breast cancer (TNBC)
Endometrial carcinoma
Soft tissue sarcoma subtypes except GIST, desmoid tumors and pleomorphic rhabdomyosarcoma

### Additional Inclusion Criteria

Able to provide informed consent, or have a legal representative able and willing to do so
≧18 years of age
Availability of a cancerous lesion amenable to biopsy and willing to undergo a pre-treatment biopsy
ECOG Performance Status of 0 or 1
Adequate bone marrow reserve as evidenced by:
   ANC>1,500/μl (unsupported by growth factors) and
   Platelet count >100,000/μl
   Hemoglobin >9 g/dL
Patients must have adequate coagulation function as evidenced by prothrombin time (PT), activated partial thromboplastin time (aPTT) and international normalized ratio (INR) within normal institutional limits
Adequate hepatic function as evidenced by:
   Serum total bilirubin ULN
   Aspartate aminotransferase (AST) and alanine aminotransferase (ALT) ≦2.5×ULN.
   Alkaline phosphatase ≦2.5×ULN, unless the elevated alkaline phosphatase is due to bone metastasis.
   In case alkaline phosphatase is >2.5×ULN patients are eligible for inclusion if aspartate aminotransferase (AST) and alanine aminotransferase (ALT) ≦1.5×ULN
Adequate renal function as evidenced by a serum/plasma creatinine <1.5×ULN
Recovered from the effects of any prior surgery, radiotherapy or other antineoplastic therapy to CTCAE v4.03 grade 1, baseline or less, except for alopecia
Women of childbearing potential or fertile men and their partners must be willing to abstain from sexual intercourse or to use an effective form of contraception during the study and for 6 months following the last dose of EphA2-targeted docetaxel liposome. Acceptable methods of effective contraception besides true abstinence include: 1) established use of oral, injected or implanted hormonal methods of contraception, 2) placement of an intrauterine device (IUD) or intrauterine system (IUS), barrier methods of contraception, including condom or occlusive cap with spermicidal foam/gel/cream/suppository, 3) male sterilization with appropriate post vasectomy documentation of the absence of sperm in the ejaculate (for female patients on the study, the vasectomized male partner should be the sole partner for that subject)

### Exclusion Criteria

Prior treatment with docetaxel within 6 months of study enrollment
Pregnant or lactating
Treatment with systemic anticoagulation (e.g. warfarin, heparin, low molecular weight heparin, anti-Xa inhibitors, etc.) except aspirin
Any evidence of hematemesis, melena, hematochezia, grade 2 hemoptysis, or gross hematuria
Any history of hereditary bleeding disorders
Presence of an active infection or with an unexplained fever >38.5° C. during screening visits or on the first scheduled day of dosing, which in the investigator's opinion might compromise the patient's participation in the trial or affect the study outcome. At the discretion of the investigator, patients with tumor fever may be enrolled
Known CNS metastases p1 Known hypersensitivity to the components of EphA2-targeted docetaxel liposome, or docetaxel
Prior treatment with EphA2-targeted docetaxel liposome
Received treatment, within 28 days or 5 half-lives, whichever is shorter, prior to the first scheduled day of dosing, with any investigational agents that have not received regulatory approval for any indication or disease state and all prior clinically significant treatment related toxicities have resolved to Grade 1 or baseline
Received other recent antitumor therapy including any standard chemotherapy or radiation within 14 days (or have not yet recovered from any actual toxicities of the most recent therapy) prior to the first scheduled dose of EphA2-targeted docetaxel liposome
Received any anti-cancer drug known to have anti-VEGF/VEGFR activity within a period of 5 half-lives of this drug (e.g. 100 days for bevacizumab, 75 days for ramucirumab) prior to the first scheduled dose of EphA2-targeted docetaxel liposome
Clinically significant cardiac disease, including: NYHA Class Ill or IV congestive heart failure, unstable angina, acute myocardial infarction within six months of planned first dose, arrhythmia requiring therapy (including torsades de pointes, with the exception of extrasystoles, minor conduction abnormalities, or controlled and well treated chronic atrial fibrillation)
Patients who are not appropriate candidates for participation in this clinical study for any other reason as deemed by the investigator
Patients who received organ or allogeneic bone marrow or peripheral blood stem cell transplants
Chronic use of corticosteroids more than 10mg daily prednisone equivalent during the past 4 weeks prior to planned start of EphA2-targeted docetaxel liposome
Concomitant use of strong inhibitors of CYP3A
Patients with peripheral neuropathy of grade 2 or higher

1. An EphA2-targeted docetaxel-generating liposome comprising a docetaxel-generating prodrug encapsulated within a lipid vesicle comprising one or more lipids, a PEG lipid derivative and an EphA2 binding moiety on the outside of the lipid vesicle.

2. The liposome of claim 1, wherein the EphA2 binding moiety is a scFv moiety covalently bound to a lipid within the lipid vesicle.

3. The liposome of claim 1, wherein the docetaxel generating prodrug is a compound of Formula (I) 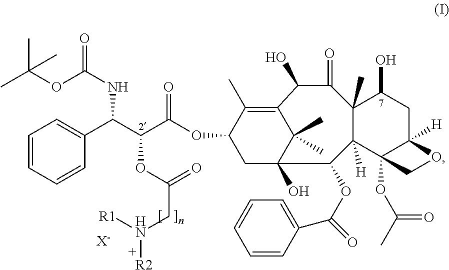
where R1 and R2 are each independently H or lower alkyl, and n is an integer 2-3.

4. The liposome of claim 1, wherein the EphA2 binding moiety is a scFv moiety comprising the CDRs of SEQ ID NO:40 or SEQ ID NO:41.

5. The liposome of claim 4, wherein the EphA2 binding moiety is a scFv moiety comprising the sequence of SEQ ID NO:41.

6. The liposome of claim 1, wherein the lipid vesicle comprises sphingomyelin, cholesterol and PEG-DSG.

7. The liposome of claim 6, wherein the lipid vesicle comprises sphingomyelin, cholesterol and PEG-DSG in a weight ratio of about 4.4:1.6:1.

8. The liposome of claim 1, wherein the liposome encapsulates a docetaxel-generating prodrug of formula (I) 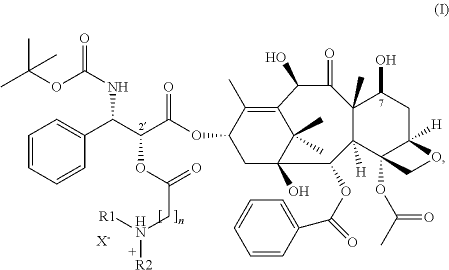
where R1 and R2 are each C₁-C₃ alkyl, and n is 2 or 3.

9. The liposome of claim 1, wherein the docetaxel-generating prodrug encapsulates the compound of formula (I) with sucrose octasulfate.

10. The liposome of claim 1, wherein the docetaxel-generating prodrug is a sucrose octasulfate salt of any one of Compounds 1-3 encapsulated in a liposome.

11. The liposome of claim 1, wherein the liposome further comprises the scFv moiety covalently bound to PEG-DSPE as scFv-PEG-DSPE in a ratio of about 1:142 by weight with respect to the total sphingomyelin in the liposome.

12. The liposome of claim 1 where the liposome has a drug-to-phospholipid ratio in the liposome greater than 250 g docetaxel equivalents/mol phospholipid, and preferably between 250-350 g/mol when the drug loading is based on docetaxel equivalents.

13. The liposome of claim 1 where the pH of the storage buffer is below neutral, preferably between 4-6.5, and more preferably between 5-6.

14. The liposome of claim 1, wherein the EphA2 binding moiety binds to the same epitope on EphA2 as an scFv consisting of SEQ ID NO:41.

15. The liposome of claim 1, wherein the EphA2 binding moiety competes for binding to EphA2 with an scFv consisting of SEQ ID NO:41.

16. A method of treating cancer in a human patient need thereof, the method comprising administering to the human patient a therapeutically effective amount of the liposome of claim 1 in a pharmaceutical composition.

17. The method of claim 16, wherein the cancer is selected from the group consisting of breast, solid tumors, gastric, esophageal, lung, prostate and ovarian cancer.

18. The method of claim 16, wherein the cancer is triple negative breast cancer (TNBC).

19. The method of claim 16, wherein the cancer is gastric, gastroesophageal junction or esophageal carcimona.

20. The method of claim 16, wherein the cancer is small cell lung cancer or non-small cell lung cancer.

21. The method of claim 16, wherein the cancer is ovarian cancer, endometrial carcinoma or urothelial carcinoma.

22. The method of claim 16, wherein the cancer is prostate adenocarcinoma.

23. The method of claim 16, wherein the cancer is soft tissue carcinoma or squamous cell carcinoma of the head and neck (SCCHN).

24. The method of claim 16, wherein the cancer is Pancreatic ductal adenocarcinoma (PDAC).

25. The method of claim 16, wherein the liposome comprises an EphA2 binding scFv moiety comprising the sequence of SEQ ID NO:41.

26. The method of claim 16, wherein the liposome comprises the docetaxel-generating prodrug selected from the group consisting of: Compound 1, Compound 2, Compound 3, Compound 4, Compound 5, and Compound 6.

27. The method of claim 26, wherein the docetaxel prodrug is Compound 3.

28. The method of claim 26, wherein the docetaxel prodrug is Compound 6.

29. The liposomes of claim 1 where the hematologic toxicity is less than that of free docetaxel.

30. The method of claim 14, where the hematologic toxicity of the dose of docetaxel delivered in the liposome is less than that of the same dose of free docetaxel.


SEQUENCE LISTING (56 sequences)

SEQ ID NO: 1 - protein, 30 aa, Artificial Sequence
  Synthetically Generated Sequence
QVQLVQSGGGLVQPGGSLRLSCAASGFTFS

SEQ ID NO: 2 - protein, 14 aa, Artificial Sequence
  Synthetically Generated Sequence
WVRQAPGKGLEWVT

SEQ ID NO: 3 - protein, 32 aa, Artificial Sequence
  Synthetically Generated Sequence
RFTISRDNSKNTLYLQMNSLRAEDTAVYYCAR

SEQ ID NO: 4 - protein, 11 aa, Artificial Sequence
  Synthetically Generated Sequence
WGQGTLVTVSS

SEQ ID NO: 5 - protein, 22 aa, Artificial Sequence
  Synthetically Generated Sequence
SSELTQPPSVSVAPGQTVTITC

SEQ ID NO: 6 - protein, 15 aa, Artificial Sequence
  Synthetically Generated Sequence
WYQQKPGTAPKLLIY

SEQ ID NO: 7 - protein, 32 aa, Artificial Sequence
  Synthetically Generated Sequence
GVPDRFSGSSSGTSASLTITGAQAEDEADYYC

SEQ ID NO: 8 - protein, 11 aa, Artificial Sequence
  Synthetically Generated Sequence
FGGGTKLTVLG

SEQ ID NO: 9 - protein, 5 aa, Artificial Sequence
  Synthetically Generated Sequence
SYAMH

SEQ ID NO: 10 - protein, 16 aa, Artificial Sequence
  Synthetically Generated Sequence
VISPAGNNTYYADSVK

SEQ ID NO: 11 - protein, 16 aa, Artificial Sequence
  Synthetically Generated Sequence
VISPAGRNKYYADSVK

SEQ ID NO: 12 - protein, 17 aa, Artificial Sequence
  Synthetically Generated Sequence
VISPDGHNTYYADSVKG

SEQ ID NO: 13 - protein, 16 aa, Artificial Sequence
  Synthetically Generated Sequence
VISPHGRNKYYADSVK

SEQ ID NO: 14 - protein, 16 aa, Artificial Sequence
  Synthetically Generated Sequence
VISRRGDNKYYADSVK

SEQ ID NO: 15 - protein, 16 aa, Artificial Sequence
  Synthetically Generated Sequence
VISNNGHNKYYADSVK

SEQ ID NO: 16 - protein, 16 aa, Artificial Sequence
  Synthetically Generated Sequence
VISPAGPNTYYADSVK

SEQ ID NO: 17 - protein, 16 aa, Artificial Sequence
  Synthetically Generated Sequence
VISPSGHNTYYADSVK

SEQ ID NO: 18 - protein, 16 aa, Artificial Sequence
  Synthetically Generated Sequence
VISPNGHNTYYADSVK

SEQ ID NO: 19 - protein, 16 aa, Artificial Sequence
  Synthetically Generated Sequence
AISPPGHNTYYADSVK

SEQ ID NO: 20 - protein, 16 aa, Artificial Sequence
  Synthetically Generated Sequence
VISPTGANTYYADSVK

SEQ ID NO: 21 - protein, 16 aa, Artificial Sequence
  Synthetically Generated Sequence
VISPHGSNKYYADSVK

SEQ ID NO: 22 - protein, 16 aa, Artificial Sequence
  Synthetically Generated Sequence
VISNNGHNTYYADSVK

SEQ ID NO: 23 - protein, 16 aa, Artificial Sequence
  Synthetically Generated Sequence
VISPAGTNTYYADSVK

SEQ ID NO: 24 - protein, 16 aa, Artificial Sequence
  Synthetically Generated Sequence
VISPPGHNTYYADSVK

SEQ ID NO: 25 - protein, 16 aa, Artificial Sequence
  Synthetically Generated Sequence
VISHDGTNTYYADSVK

SEQ ID NO: 26 - protein, 16 aa, Artificial Sequence
  Synthetically Generated Sequence
VISRHGNNKYYADSVK

SEQ ID NO: 27 - protein, 17 aa, Artificial Sequence
  Synthetically Generated Sequence
VISYDGSNKYYADSVKG

SEQ ID NO: 28 - protein, 11 aa, Artificial Sequence
  Synthetically Generated Sequence
ASVGATGPFDI

SEQ ID NO: 29 - protein, 11 aa, Artificial Sequence
  Synthetically Generated Sequence
QGDSLRSYYAS

SEQ ID NO: 30 - protein, 7 aa, Artificial Sequence
  Synthetically Generated Sequence
GENNRPS

SEQ ID NO: 31 - protein, 12 aa, Artificial Sequence
  Synthetically Generated Sequence
NSRDSSGTHLTV

SEQ ID NO: 32 - protein, 23 aa, Artificial Sequence
  Synthetically Generated Sequence
ASTGGGGSGGGGSGGGGSGGGGS

SEQ ID NO: 33 - protein, 20 aa, Artificial Sequence
  Synthetically Generated Sequence
GGGGSGGGGSGGGGSGGGGS

SEQ ID NO: 34 - protein, 15 aa, Artificial Sequence
  Synthetically Generated Sequence
GGGGSGGGGSGGGGS

SEQ ID NO: 35 - protein, 23 aa, Artificial Sequence
  Synthetically Generated Sequence
ASTGGGGAGGGGAGGGGAGGGGA

SEQ ID NO: 36 - protein, 20 aa, Artificial Sequence
  Synthetically Generated Sequence
GGGGAGGGGAGGGGAGGGGA

SEQ ID NO: 37 - protein, 24 aa, Artificial Sequence
  Synthetically Generated Sequence
TPSHNSHQVPSAGGPTANSGTSGS

SEQ ID NO: 38 - protein, 18 aa, Artificial Sequence
  Synthetically Generated Sequence
GGSSRSSSSGGGGSGGGG

SEQ ID NO: 39 - protein, 457 aa, Artificial Sequence
  Synthetically Generated Sequence
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 259 aa, Artificial Sequence
  Synthetically Generated Sequence
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 41 - protein, 259 aa, Artificial Sequence
  Synthetically Generated Sequence
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLGGGSGGC

SEQ ID NO: 42 - protein, 214 aa, Artificial Sequence
  Synthetically Generated Sequence
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 44 - protein, 259 aa, Artificial Sequence
  Synthetically Generated Sequence
QVQLVQSGGGLVQPGGSLRLSCAASGFTFSSYAMHWVRQAPGKGLEWVSVISPAGRNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 45 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggccgcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 46 - protein, 259 aa, Artificial Sequence
  Synthetically Generated Sequence
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 47 - DNA, 861 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccggatggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtcgtacggtggcggcgcccagtcaccatcatcatcaccactgataa

SEQ ID NO: 48 - protein, 259 aa, Artificial Sequence
  Synthetically Generated Sequence
QVQLVQSGGGLVQPGGSLRLSCAASGFTFSSYAMHWVRQAPGKGLEWVTVISPSGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 49 - DNA, 840 bp, Artificial Sequence
  Snythetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgagcggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 50 - protein, 259 aa, Artificial Sequence
  Synthetically Generated Sequence
QVQLVQSGGGLVQPGGSLRLSCAASGFTFSSYAMHWVRQAPGKGLEWVTVISPNGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 51 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaacggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 52 - protein, 259 aa, Artificial Sequence
  Synthetically Generated Sequence
QVQLVQSGGGLVQPGGSLRLSCAASGFTFSSYAMHWVRQAPGKGLEWVSAISPPGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 53 - DNA, 816 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgcgattagcccgccgggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 54 - protein, 259 aa, Artificial Sequence
  Synthetically Generated Sequence
QVQLVQSGGGLVQPGGSLRLSCAASGFTFSSYAMHWVRQAPGKGLEWVTVISPTGANTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 55 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaccggcgcgaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 56 - DNA, 840 bp, Artificial Sequence
  Synthetically Generated Sequence
atgggctggtctctgatcctgctgttcctggtggccgtggccacgcgtgtgctctcgcaagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa